(19) United States
(12) Patent Application Publication
(10) Pub. No.: US 2010/0048549 A1
(43) Pub. Date: Feb. 25, 2010

(54) Peptidomimetics selective for the somatostatin receptor subtypes 1 and/or 4

(75) Inventors: Jussi Tomperi, Turku (FI); Harri Salo, Turku (FI); Oili Kallatsa, Turku (FI); Pia Knuuttila, Turku (FI); Päivi Laakso, Ilmarinen (FI); Iisa Höglund, Turku (FI); Anna-Marja Hoffrén, Raisio (FI); Kurt Kokko, Hyvinkää (FI); Pauli Saarenketo, Piikkiö (FI); Mia Engström, Espoo (FI); Siegfried Wurster, Piikkiö (FI)

Correspondence Address: OLIFF & BERRIDGE, PLC, P.O. BOX 320850, ALEXANDRIA, VA, 22320-4850, US

(73) Assignee: Siegfried Wurster, Piikkiö (FI)

(21) Appl. No.: 11/919,968

(22) PCT Filed: May 16, 2006
(86) PCT No.: PCT/FI2006/050197

§ 371 Date: Oct. 15, 2009

Related U.S. Application Data
(60) Provisional application No. 60/682,020, filed on May 18, 2005.

(30) Foreign Application Priority Data
May 18, 2005 (FI) 20055235

(51) Int. Cl. A61K 31/18; A61K 31/5375; A61K 31/472; A61K 31/445; A61K 31/40; C07D 295/13; C07D 217/02; C07D 211/28; C07D 207/09; C07C 311/13; A61P 29/00; A61P 25/00; A61P 25/24; A61P 35/00; A61P 9/00; A61P 17/02; A61P 27/02

(52) U.S. Cl. 514/231.2; 514/307; 514/319; 514/408; 514/602; 544/160; 546/139; 546/206; 548/400; 564/84

(57) ABSTRACT

The invention relates to (hetero)arylsulfonylamino based peptidomimetics of formula (I), wherein A, D, E, J, Q1 R1, R2, R3, p and j are defined as disclosed, or a pharmaceutically acceptable salt or ester thereof. Compounds of formula (I) possess high affinity and selectivity for the somatostatin receptor subtypes sst₁ and/or sst₄ and can be used for the treatment or diagnosis of diseases or conditions wherein sst₁ and/or sst₄ agonists or antagonists are indicated to be useful.

### FIELD OF THE INVENTION

The current invention relates to (hetero)arylsulfonylamino based peptidomimetics useful for preventing, treating or diagnosing medical disorders related to somatostatin receptor subtypes 1 and/or 4.

### BACKGROUND OF THE INVENTION

Somatostatin, or somatotropin-release inhibitory factor (SRIF), is a cyclic peptide found in two major endogenous forms in humans, one of them is made up of 14 (SRIF-14) and the other one of 28 (SRIF-28) amino acids. The shorter SRIF-14 is identical in sequence to the C-terminal half of SRIF-28. In addition, there is a third endogenous human peptide called cortistatin, for which so far no dedicated receptor has been identified but which shares a high degree of sequence similarities with SRIF-14 and which possesses similar affinities towards the five human somatostatin subtypes as SRIF-14.

Somatostatin is produced widely in the human body and acts both systemically and locally to inhibit the secretion of various hormones, growth factors and neurotransmitters. The pepdie is thus directly or indirectly involved in the regulation of processes such as for example cellular proliferation, glucose homeostasis, inflammation and pain. The effects of somatostatin are mediated by a family of G protein-coupled receptors, of which five subtypes (sst₁₋₅) have been cloned in humans (Reisine and Bell 1995; Patel 1999). The affinities of the two endogenous SRIF peptides on the five subtypes are relatively similar, with the exception that SRIF-28 has been reported to have a moderate preference for the sst₅. Nonetheless, the five subtypes possess different tissue expression profiles and do also show some differences in their usage of signalling pathways. The pleiotropic physiological responses produced by somatostatin are thus a reflection of its widespread distribution, the existence of multiple receptor subtypes and the differential coupling of these subtypes to intracellular signalling pathways.

Based on sequence similarities and affinities towards a number of octapeptide and hexapeptide analogues of somatostatin, the five somatostatin receptor subtypes have been divided into two subfamilies: one made up of sst₂, sst₃ and sst₅ and a second one consisting of sst₁ and sst₄. The former subfamily possesses high affinities towards these hexapeptide and octapeptide analogues, whereas the latter subfamily interacts with them only in a rather poor manner (Hoyer et al. 1995). Due to the availability of the aforementioned high affinity ligands with selectivity for the subtypes sst_{2,3,5}, the physiology of this subfamily has been characterized much more thoroughly and it appears that most of the ‘classical’ effects of somatostatin, such as its very potent inhibition of growth hormone, insulin, glucagon and gastric acid release, are mediated either exclusively or primarily via members of this subfamily.

Nonetheless, while the physiology and pathophysiology of the subtypes sst₁ and sst₄ is less well understood, there have been a number of findings described in scientific publications and the patenting literature about the role of these subtypes. For example, U.S. Pat. No. 6,124,256 reported that given their localisation in the vascular wall and their time-related induction during the proliferative stage, the sst₁ and/or the sst₄ may be the optimal subtypes to prevent fibroproliferative vasculopathy via somatostatin receptor-based therapies. In agreement with this, Curtis et al. (2000) have described the sst₁ and the sst₄ to represent the predominant subtypes expressed in human blood vessels and have proposed the use of sst₁- or sst₄-selective agonists for the treatment of proliferative diseases involving endothelial cells. Aavik et al. (2002) have demonstrated the purportedly sst₁- and sst₄-selective peptide CH-275 to be able to prevent intimal hyperplasia after rat carotid denudation injury. Taken together, these findings may explain why two peptide analogues of somatostatin which possess very high preferences for the subtypes sst₂ and sst₅, but have rather low affinities for the subtypes sst₁ or sst₄, namely octreotide and lanreotide, failed to show efficacy in clinical trials aiming at the prevention of restenosis after percutaneous transluminal angioplasty (Eriksen et al. 1995; van Essen et al. 1997).

Due to the fact that the activation of the sst₁ subtype causes antiproliferative effects, sst₁-selective agonist may be useful for the treatment of tumours bearing this subtype. In that regard it is of interest to note that sst₁ receptor have been described to be expressed in prostate cancer (Sinisi et al. 1997; Reubi et al. 1997; Reubi et al. 2001), but not in normal prostate tissue.

WO 97/03054 and U.S. Pat. No. 6,221,870 describe benzo[g]quinoline-derived (WO 097/03054) or ergoline-derived (U.S. Pat. No. 6,221,870) sst₁-selective antagonist as lowering aggressive behaviour in mice and consequently suggest such compounds to be useful for the treatment of depression, anxiety, affective disorders and attention deficit and hyperactivity disorders (ADHD).

According to Bito et al. (1994) the sst₄ subtype is expressed at high levels in the rat hippocampus where somatostatin has been reported to play a significant role in the regulation of membrane conductance. Since the hippocampus is a brain structure closely linked to learning and memory, as well as mental disorders such as depression and schizophrenia, the prominent presence of the sst₄ subtype in this brain area suggests that sst₄ selective agonists or antagonists with the ability to pass the blood-brain-barrier may have considerable therapeutic potential in learning and memory.

Employing in situ hybridisation, Mori et al. (1997) have shown that in the rat eye sst₄ expression predominates in the posterior iris epithelium and ciliary body and in addition, the authors observed somatostatin to lower intraocular pressure (iop). Based on these observations they suggested that sst₄-selective ligands may be useful as anti-glaucoma agents.

During the last 10 to 15 years peptide receptor have gained increasing importance for diagnostic purposes, in particular the in vivo targeting of human cancers. The basis for this role rests on the observation that certain tumours express large quantities of such peptide receptor, with somatostatin representing the ‘paradigmatic’ case (Reubi, 2002). Thus, in vivo somatostatin receptor scintigraphy has been proven to be a sensitive and valuable non-invasive technique, which does hot only allow for the localization, differential diagnosis and postoperative follow-up of tumours and their metastases (Haldeman et al., 1995), but does also offer a tool to predict the outcome of somatostatin analogue treatment (Janson et al. 1994) and a tool to adjust the treatment protocol of patients according to their disease stage (van den Anker-Lugtenburg et al. 1996). Most of the radiolabelled ligands that have so far been developed for somatostatin receptor scintigraphy are based on the octapeptide octreotide, which is selective for the subtypes sst₂, sst₃ and sst₅, but does hot permit the visualization of sst₁ and sst₄ receptor. However, this would be highly desirable, as there are forms of tumour that predominately express one of these two subtypes, such as for example prostate cancer (Reubi, 2002). In addition, there are human tissues where one of these two subtypes either clearly predominates, e.g. the sst₁ in human blood vessels (Curtis et al, 2000), or even represents the sole somatostatin receptor present, e.g. the sst₄ in the lung (Fehlmann et al. 2000). The ability to visualize sst₁ and/or sst₄ receptor via the use of subtype selective radiolabelled ligands would therefore not only open up as yet unavailable diagnostic options for tumours bearing these receptor subtypes, but would potentially also allow the diagnostic imaging of tissues for other purposes, such as for example the visualization of blood vessels in arteriosclerosis or in suspected cases of cerebral aneurysm.

The endogenous somatostatin peptides have a very short biological half-life and are therefore not well suited for therapeutic use. A number of shorter hexa- and octapeptide analogues of somatostatin with improved biological stability have been identified (e.g. U.S. Pat. No. 4,485,101, U.S. Pat. No. 5,409,894 or WO 97/47317). However, as mentioned above, these abbreviated peptide analogues are heavily biased in favour of the sst_{2,3,5} subfamily and do not show much interaction with the subtypes sst₁ or sst₄. In contrast, WO 97/14715 and Rivier et al. (2001) describe a group of sst₁ preferring undecapeptide agonists. However, besides their often rather short biological half-lifes, peptides also possess other unsatisfactory properties, which make them problematic as medicines. For example, peptides have a very limited ability to penetrate biological membranes, which is one of the reasons why it is very rarely feasible to give peptides via an oral route and why peripherally applied peptides generally do not reach the central nervous system.

In recent years, a number of nonpeptide somatostatin agonists have also been identified. Besides the already mentioned sst₁-selective antagonists reported in WO 97/03054 and U.S. Pat. No. 6,221,870, the patent WO 97/43278 describes a number of thiourea-based compounds that preferentially interact with the somatostatin receptor subtype sst₄ and the histamine receptor subtype H₃. U.S. Pat. No. 6,329,389 and U.S. Pat. No. 6,352,982 provide sst₄-selective compounds centred on tetrahydroquinoline or 4,1-benzoxazepine scaffolds. Embarking from a generally accepted hypothesis on the structure-activity-relationship of somatostatin receptor active compounds, namely the assumption that the amino acid residues 8 and 9 in SRIF-14 (which consist of a tryptophan and a lysine) are essential for proper ligand-receptor interaction, and employing a mix-and-split combinatorial chemistry strategy, Rohrer et al. (1998) have been able to identify subtype-selective agonists for each of the five human somatostatin receptor subtypes.

The current invention describes novel ligands for the somatostatin receptor subtypes sst₁ and/or sst₄. These compounds are sulfonamido-peptidomimetics and are in part related to similar compounds presented in the patent applications PCT/FI2004/000584 and PCT/FI2004/000585. To some extent related monocyclic or bicyclic sulfonamide derivatives have also been described in a number of scientific publications and patents, albeit not as agonists or antagonists of somatostatin receptor. Particularly well represented among these publications and patents are thrombin and serine protease inhibitors which are featured in CN 1183766, DE 19548797, DE 3942114, DE 442-4828, EP 555824, EP 565396, EP 739886, U.S. Pat. No. 5,248,673, WO 9208709, as well as in Kobe J Med Sci (1980), 26(1):1-9; Pharmazie (1982), 37(1):13-16; Pharmazie (1982), 36(9):597-603; Pharmazie (1982), 37(3):178-82; Pharmazie (1983), 38(11):793; Bioorg & Med Chem Left (1995), 3(8):1145-56 and Bioorg & Med Chem Left (2001), 11(14):1947-50. US 20030166652 teaches on ligands for CCR3 receptor, WO 2004101507 on N-sulfonylated amino acid derivatives as inhibitors of matriptase in the treatment of cancer, WO 2003070229 on urokinase inhibitors, U.S. Pat. No. 5,244,895 on anti-ulcer agents, DE 3942114 on blood vessel relaxants, WO 2002100848 on sigma receptor ligands, EP 109023 on vasodilators and hypotensors, JP 11228547 on the production of 6-amino-1,4-dialkylhexahydro-1H-1,4-diazepine derivatives, WO 97/29097 on 5-HT₇ antagonists, WO 01/34562 on compounds with calcimimetic activity, WO 2004/014844 on compounds that inhibit factor IX (thereby preventing blood coagulation), WO 2004/113280 on inhibitor for the neuro-transmitter transporter GlyT1 and WO 9305014 on aromatic sulfonamide derivatives that inhibit Ca²⁺-dependent enzymes and proteins, while J Biosci (1985), 40C(9-10):612-616 deals with fungal mycosporines and Int J Pept Prot Res (1984), 24(4):347-58 describes SFP and ELP inhibitors. Finally, WO 9005739 reports on the carboxy-terminal sequencing of proteins and peptides using novel coupling reagents, PNAS (1978), 75(9):4115-19 on the chemical determination of polypeptide hormones, JACS (1996), 118(48): 12004-11 on a fluorescent assay for recombinases and topoisomerases, and Appl Biochem and Biotech (1994), 47(2-3):277-92 on antibody-catalyzed primary amide hydrolysis.

### SUMMARY OF THE INVENTION

The present invention relates to non-peptide compounds endowed with a high degree of selectivity towards the two somatostatin receptor subtypes sst₁ and or sst₄ and their use. The scope of the invention is summarized in the independent claims.

It will be appreciated by those skilled in the art that a wide variety of therapeutic, prophylactic and diagnostic applications may be prepared from the compounds of the current invention based on the agonist or antagonist nature of these compounds towards the sst₁ and/or the sst₄ receptor:

1. Compounds of the invention are useful for the prevention or treatment of diseases or symptoms of anxiety, depression, schizophrenia, epilepsy, attention deficit and hyperactive disorders and neurodegenerative diseases such as dementia, Alzheimer's disease and Parkinson's disease. The treatment of affective disorders includes bipolar disorders, e.g. manic-depressive psychoses, extreme psychotic states, e.g. mania and excessive mood swings for which a behavioural stabilization is being sought. The treatment of anxiety states includes generalized anxiety as well as social anxiety, agoraphobia and those behavioural states characterized by social withdrawal, e.g. negative symptoms.

2. Compounds of the invention, depending on their agonistic or antagonistic character on the sst₁ or sst₄, are advantageous in diseases involving pathological vascular proliferation, e.g. angiogenesis, restenosis, smooth muscle proliferation, endothelial cell proliferation and new blood vessel sprouting or conditions requiring the activation of neovascularization. The angiogenic disease may for example be age-related macular degeneration or vascular proliferation associated with surgical procedures, e.g. angioplasty and AV shunts. Other possible uses are the treatments of arteriosclerosis, plaque neovascularization, hypertrophic cardiomyopathy, myocardial angiogenesis, valvular disease, myocardiac infarction, coronary collaterals, cerebral collaterals and ischemic limb angiogenesis.

3. Compounds of the invention are also indicated for the treatment of diseases connected to pathological condition in the retina and/or iris-ciliary body of mammals. Such conditions may be high intraocular pressure (IOP) and/or deep ocular infections. Treatable diseases may e.g. be glaucoma, stromal keratitis, iritis, retinitis, cataract and conjunctivitis. Other diseases connected to the eye may be ocular and corneal angiogenic conditions, for example, corneal graft rejection, retrolental fibroplasia, Osler-Webber Syndrome or rubeosis.

4. Compounds of the invention are also useful for the prevention or treatment of diseases or symptoms connected to diabetic complications such as diabetic retinopathy, diabetic nephropathy, diabetic neuropathy, Doan syndrome and orthostatic hypotension.

5. Compounds of the invention are useful for the treatment of a number of tumours such as e.g. the proliferation of adenoma cells, thyroid cancer, large bowel cancer, breast cancer, prostatic cancer, small cell lung cancer, non-small cell cancer, pancreatic cancer, stomach cancer, GI tumours, cholangiocarcinoma, hepatic cancer, vesical cancer, ovarian cancer, melanoma, osteosarcoma, chondrosarcoma, malignant pheochromocytoma, neuroblastoma, brain tumours, thymoma, paragangliomas, prostate carcinomas, sarcomas, gastroenteropancreatic tumours, gastric carcinomas, phaeochromocytomas, ependymomas, renal cancers, leukemia e.g., leukemia of basophilic leukemia, chronic lymphocytic leukemia, chronic myeloid leukemia, Hodgkin disease and non-Hodgkin lymphoma.

6. Compounds of the invention, after incorporation of a label (e.g. 35-S, 123-I, 125-I, 111-In, 11-C, etc.) either directly in the compound or via a suitable spacer, can also be used for the imaging of healthy or diseased tissues and/or organs, such as prostate, lung, brain, blood vessels or tumours possessing sst₁ and/or sst₄ receptors.

7. Compounds of the invention are useful for targeting tumours with sst₁ and/or sst₄ receptors using a compound of the invention conjugated with anti-cancer drugs directly or using a suitable spacer.

8. Finally, compounds of the invention are useful for wound healing, ovulation, menstruation, placentation, peptic ulcers, psoriasis, rheumatoid arthritis and Crohn's disease.

### DETAILED DESCRIPTION OF THE INVENTION

The invention relates to the use of compounds having general formula I and pharmaceutically acceptable salts and esters thereof for the preparation of a medicament for treating a disease or condition in mammals where an agonist or antagonist of somatostatin receptor subtypes 1 and/or 4 is indicated to be useful,

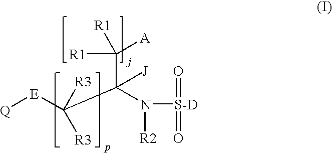

wherein

A is NR6R6 or NR6-(C₁-C₃)alkyl-NR6R6 and the (C₁-C₃)alkyl may be unsubstituted or substituted with one to four groups selected from R^{a}; or

A is a 5- to 6-membered saturated or unsaturated ring containing 0 to 2 nitrogens, the said ring being unsubstituted or substituted with 1 to 3 groups independently selected from R6 and —(CH₂)ₛ—NR6R6; or

A and J together with the carbon atom to which they are attached form a 5- to 6-membered ring containing 1 to 2 nitrogens, said ring being unsubstituted or substituted with 1 to 3 groups independently selected from R6 or —(CH₂)ₛ—NR6R6; or

A and J together with the carbon atom to which they are attached form a 5- to 6-membered ring containing 0 nitrogens, said ring being substituted by a group —(CH₂)ₛ—NR6R6 and 0 to 2 groups independently selected from R6; or

A and R2 together with the atoms to which they are attached form a saturated 5- or 6-membered ring, said ring being substituted by a group —(CH₂)ₛ—NR6R6 and 0 to 3 groups independently selected from (C₁-C₆)alkyl;

D is aryl, heteroaryl or aryl-(C₁-C₂)-alkyl and may be unsubstituted or substituted with one to seven groups selected from R^{a};

E is O, S, NR^{b} or CR^{b}R^{b};

J is H or methyl; or J is part of a spiro ring system together with A;

Q is

1. aryl,

2. heteroaryl or

3. a group of formula

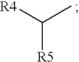

wherein the aryl or heteroaryl is unsubstituted or substituted with 1 to 4 substituents selected from R^{a};

R1 is independently

a group selected from R^{a}; or

R1 and R1 together form ═O,

R2 is

1) H,

2) (C₁-C₆)alkyl,

3) (C₂-C₆)alkenyl,

4) (C₃-C₇)cycloalkyl, or

5) benzyl

or R2 is part or a ring system together with A;

R3 is independently

1) H,

2) (C₁-C₆)alkyl, or

when E is NR^{b} or CR^{b}R^{b}, R3 and R^{b} can form a double bond between the atoms to which they are attached;

R4 is

1) H,

2) (C₁-C₆)alkyl,

3) (C₂-C₆)alkenyl,

4) (C₂-C₆)alkynyl,

5) Cy,

6) Cy-(C₁-C₆)alkyl or

7) Cy-(C₂-C₆)alkenyl

wherein alkyl, alkenyl, alkynyl and Cy are each optionally substituted with one to two substituents selected from R^{a};

R5 is

1) H,

2) (C₁-C₆)alkyl,

3) (C₂-C₆)alkenyl,

4) (C₂-C₆)alkynyl

5) aryl,

6) aryl-(C₁-C₆)alkyl,

7) heteroaryl,

8) heteroaryl-(C₁-C₆)alkyl,

9) —OR^{b};

10) —(CH₂)ₖ—OR^{b} or

11) —(CH₂)ₖC(O)NHR^{b},

wherein aryl and heteroaryl are each optionally substituted with one to two substituents selected from R^{a}; or

R4 and R5 together with the atom to which they are attached form a 3- to 7-membered ring containing 0 to 2 heteroatoms selected from N, O and S, wherein the said ring can be substituted with one to three substituents selected from R^{a}; or the said ring can be fused to aryl or heteroaryl which may be substituted with one to three substituents selected from R^{a};

R6 is independently

1) H,

2) (C₁-C₆)alkyl,

3) (C₃-C₇)cycloalkyl,

4) (C₃-C₇)cycloalkyl(C₁-C₆)alkyl or

5) —C(═NR^{b})NR^{b}R^{b},

wherein symbols R^{b} together may form a 5- to 6-membered unsaturated or saturated ring; or

R6 and R6 together with the atoms to which they are attached form a 5- to 7-membered ring containing 1 to 3 heteroatoms selected from N, O and S, said ring being unsubstituted or substituted with 1 to 4 groups independently selected from (C₁-C₆)alkyl or halogen;

R^{a} is independently

1) H,

2) halogen,

3) —OR^{b},

4) —(C₁-C₆)alkyl-OR^{b},

5) (C₁-C₆)alkyl,

6) —CF₃,

7) —NO₂,

8) —SR^{b},

9) —NR^{b}R^{b},

10) —CN,

11) —C(O)R^{b},

12) (C₂-C₆)alkenyl,

13) (C₃-C₇)cycloalkyl

14) —NR^{b}C(O)R^{b} or

15) —C(O)NR^{b}.

R^{b} is independently

1) hydrogen,

2) (C₁-C₆)alkyl,

3) Cy or

4) Cy-(C₁-C₄)alkyl;

p is an integer 0 to 3;

j is an integer 0 to 4;

k is an integer 0 to 2,

s is an integer 0 to 2; and

Cy is cycloalkyl, heterocyclyl, aryl or heteroaryl, with the proviso that when E is CR^{b}R^{b} or NR^{b}, then R1 and R1 cannot together form ═O.

Moreover, the invention also relates to the use of the compounds described above for the purpose of imaging sst₁ and/or sst₄ receptor in healthy or diseased tissues and organs, such as prostate, lung, brain, blood vessels or tumours possessing sst₁ and/or sst₄ receptor, after the incorporation of a label (e.g. 35-S, 123-I, 125-I, 111-In, 11-C, etc.) either directly into the molecules or indirectly through a chelate connected via a suitable spacer.

According to another aspect, the invention also relates to compounds having the general formula (I) and pharmaceutically acceptable salts and esters thereof for the preparation of a medicament for treating a disease or condition in mammals where an agonist or antagonist of the somatostatin receptor subtypes 1 and/or 4 is indicated to be useful,

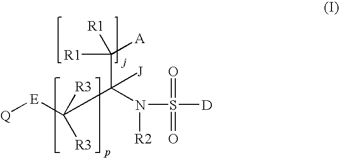

wherein

A is NR6R6 or NR6-(C₁-C₃)alkyl-NR6R6 and the (C₁-C₃)alkyl may be unsubstituted or substituted with one to four groups selected from R^{a}; or

A is a 5- to 6-membered saturated or unsaturated ring containing 0 to 2 nitrogens, the said ring being unsubstituted or substituted with 1 to 3 groups independently selected from R6 and —(CH₂)ₛ—NR6R6; or

A and J together with the carbon atom to which they are attached form a 5- to 6-membered ring containing 1 to 2 nitrogens, said ring being unsubstituted or substituted with 1 to 3 groups independently selected from R6 or —(CH₂)ₛ—NR6R6; or

A and J together with the carbon atom to which they are attached form a 5- to 6-membered ring containing 0 nitrogens, said ring being substituted by a group —(CH₂)ₛ—NR6R6 and 0 to 2 groups independently selected from R6; or

A and R2 together with the atoms to which they are attached form a saturated 5- or 6-membered ring, said ring being substituted by a group —(CH₂)ₛ—NR6R6 and 0 to 3 groups, independently selected from (C₁-C₆)alkyl;

D is aryl, heteroaryl or aryl-(C₁-C₂)-alkyl and may be unsubstituted or substituted with one to seven groups selected from R^{a};

E is O, S, NR^{b}, or CR^{b}R^{b};

J is H or methyl; or J is part of a spiro ring system together with A;

Q is

1) phenyl

2) benzyl or

3) a group of formula

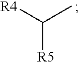

wherein phenyl or benzyl is unsubstituted or substituted with 1 to 4 substituents selected from R^{a};

R1 is independently

a group selected from R^{a};

R2 is

1) H,

2) (C₁-C₆)alkyl,

3) (C₂-C₆)alkenyl,

4) (C₃-C₇)cycloalkyl, or

5) benzyl

or R2 is part or a ring system together with A;

R3 is independently

1) H,

2) (C₁-C₆)alkyl, or

when E is NR^{b} or CR^{b}R^{b}, R3 and R^{b} can form a double bond between the atoms to which they are attached;

R4 is

1) H,

2) (C₁-C₆)alkyl,

3) (C₂-C₆)alkenyl,

4) (C₂-C₆)alkynyl,

5) Cy,

6) Cy-(C₁-C₆)alkyl or

7) Cy-(C₂-C₆)alkenyl,

wherein alkyl, alkenyl, alkynyl and Cy are each optionally substituted with one to two substituents selected from R^{a};

R5 is

1) H,

2) (C₁-C₆)alkyl,

3) (C₂-C₆)alkenyl,

4) (C₂-C₆)alkynyl

5) aryl,

6) aryl-(C₁-C₆)alkyl,

7) heteroaryl,

8) heteroaryl-(C₁-C₆)alkyl,

9) —OR^{b},

10) —(CH₂)ₖ—OR^{b} or

11) —(CH₂)ₖC(O)NHR^{b},

wherein aryl and heteroaryl are each optionally substituted with one to two substituents selected from R^{a}; or

R4 and R5 together with the atom to which they are attached form a 3- to 7-membered ring containing 0 to 2 heteroatoms selected from N, O and S, wherein the said ring can be substituted with one to three substituents selected from R^{a}; or the said ring can be fused to aryl or heteroaryl which may be substituted with one to three substituents selected from R^{a};

R6 is independently

1) H,

2) (C₁-C₆)alkyl,

3) (C₃-C₇)cycloalkyl,

4) (C₃-C₇)cycloalkyl(C₁-C₆)alkyl or

5) —C(═NR^{b})NR^{b}R^{b},

wherein symbols R^{b} together may form a 5- to 6-membered unsaturated or saturated ring; or

R6 and R6 together with the atoms to which they are attached form a 5- to 7-membered ring containing 1 to 3 heteroatoms selected from N, O and S, said ring being unsubstituted or substituted with 1 to 4 groups independently selected from (C₁-C₆)alkyl or halogen;

R^{a} is independently

1) H,

2) halogen,

3) —OR^{b},

4) —(C₁-C₆)alkyl-OR^{b},

5) (C₁-C₆)alkyl,

6) —CF₃,

7) —NO₂,

8) —SR^{b},

9) —NR^{b}R^{b},

10) —CN,

11) —C(O)R^{b},

12) (C₂-C₆)alkenyl,

13) (C₃-C₇)cycloalkyl

14) —NR^{b}C(O)R^{b} or

15) —C(O)NR^{b};

R^{b} is independently

1) hydrogen,

2) (C₁-C₆)alkyl,

3) Cy or

4) Cy-(C₁-C₄)alkyl;

p is an integer 0 to 3;

j is an integer 0 to 4;

k is an integer 0 to 2;

s is an integer 0 to 2; and

Cy is cycloalkyl, heterocyclyl, aryl or heteroaryl;

with the proviso that when

a) A contains an aromatic system, then E cannot be CR^{b}R^{b},

b) E is NR^{b} and A is NR6R6 then p and j cannot be simultaneously 1,

c) A is pyrrole or pyrazole, one of the 1 to 3 substituents on said ring must be selected from —C(═NR^{b})NR^{b}R^{b}, —(CH₂)ₛ—NR6-C(═NR^{b})NR^{b}R^{b} or —(CH₂)ₛ—NR6R6,

d) A is a 6-membered unsaturated ring, one of the 1 to 3 substituents on said ring must be selected from —C(═NR^{b})NR^{b}R^{b}, —(CH₂)ₛ—NR6-C(═NR^{b})NR^{b}R^{b} or —(CH₂)ₛ—NR6R6,

e) A is a saturated ring not containing a nitrogen atom, at least one of the 1 to 3 substituents on ring A must be selected from —C(═NR^{b})NR^{b}R^{b}, —(CH₂)ₛ—NR6-C(═NR^{b})NR^{b}R^{b} or —(CH₂)ₛ—NR6R6.

“Alkyl”, as well as other groups having the prefix “alk”, such as alkoxy, alkanoyl, means carbon chains which may be linear or branched or combinations thereof. The size of the alkyl can further be specified by adding the number of carbons in front of the group, e.g. (C₁-C₆)alkyl, (C₁-C₃)alkyl. Examples of alkyl groups include methyl, ethyl, propyl, isopropyl, butyl, sec-butyl, tert-butyl, pentyl, neo-pentyl, hexyl, heptyl, octyl, nonyl, and the like.

“Alkenyl” means carbon chains which contain at least one carbon-carbon double bond, and which may be linear or branched or combinations thereof. The size of the alkenyl can further be specified by adding the number of carbons in front of the group, e.g. (C₂-C₆)alkenyl, (C₂-C₈)alkenyl. Examples of alkenyl groups include vinyl, allyl, isopropenyl, 1-pentenyl, 2-pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. The size of the alkynyl can further be specified by adding the number of carbons in front of the group, e.g. (C₂-C₆)alkynyl, (C₂-C₈)alkynyl. Examples of alkynyl groups include ethynyl, propargyl, 3-methyl-1-pentynyl, 2-heptenyl, and the like.

“Cycloalkyl” means mono- or bicyclic saturated carbocyclic rings, each of which having 3 to 8 carbon atoms. The term also includes monocyclic rings fused to an aryl group in which the point of attachment is on the non-aromatic portion. The size of the cycloalkyl can further be specified by adding the number of carbons in front of the group, e.g. (C₃-C₇)cycloalkyl, (C₅-C₁₀)cycloalkyl. Examples of cycloalkyl groups include cyclopropyl, cyclopentyl, cyclohexyl, cycloheptyl, tetrahydronaphthyl, decahydronaphthyl, indanyl, and the like.

“Aryl” means mono- or bicyclic aromatic rings containing only carbon atoms. The term also includes aryl groups fused to a monocyclic cycloalkyl or monocyclic heterocyclyl group in which the point of attachment is on the aromatic portion. The size of the aryl can further be specified by adding the number of carbons in front of the group, e.g. (C₆-C₁₂)aryl. Examples of aryl groups include phenyl, naphthyl, indanyl, indenyl, tetrahydronaphthyl, 2,3-dihydrobenzofuranyl, benzopyranyl, 1,4-benzodioxanyl, and the like.

“Heteroaryl” means a mono- or bicyclic aromatic ring containing at least one heteroatom selected from N, O and S, with each ring containing 5 to 6 atoms. The term also includes heteroaryl groups fused to a monocyclic cycloalkyl or monocyclic heterocyclyl group in which the point of attachment is on the aromatic portion. Examples of heteroaryl groups include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3b)pyridyl, quinolyl, indolyl, isoquinolyl, and the like.

“Heterocyclyl” means mono- or bicyclic saturated rings containing at least one heteroatom selected from N, O, S, each of said rings having from 5 to 8 atoms in which the point of attachment may be carbon or nitrogen. The term also includes monocyclic heterocycles fused to an aryl or a heteroaryl group in which the point of attachment is on the non-aromatic portion. Furthermore, the term also includes partially unsaturated monocyclic rings that are not aromatic, such as 2- and 4-pyridones attached through the nitrogen. Other examples of heterocyclyl groups include pyrrolidinyl, piperidinyl, piperazinyl, imidazolinyl, 2,3-dihydrofuro(2,3-b)pyridyl, benzoxazinyl, tetrahydroquinolinyl, tetrahydroisoquinolinyl, dihydroindonyl, and the like.

The term “cycloalkyl-alkyl”, as employed herein, refers to a “cycloalkyl” as defined above, appended to the parent molecular moiety through an alkyl group as defined above. The size of the cycloalkyl and the alkyl can further be specified by adding the number of carbons in front of the group, e.g. (C₃-C₇)cycloalkyl(C₁-C₆)alkyl, (C₃-C₅)cycloalkyl(C₁-C₂)alkyl. Representative examples of cycloalkyl-alkyl include, but are not limited to, cyclohexylmethyl, 1-cyclohexylethyl, 2-cyclopentylethyl, and the like.

The term “aryl-alkyl”, as employed herein, refers to an “aryl” as defined above, appended to the parent molecular moiety through an (C₁-C₆)alkyl group as defined above. The size of the aryl or alkyl can further be specified by adding the number of carbons in front of the group, e.g. aryl(C₁-C₆)alkyl, (C₆-C₁₂)aryl-(C₁-C₃)alkyl. Representative examples of aryl-alkyl include, but are not limited to, 2-naphthylmethyl, 1-(2-indanyl)ethyl, 2-tetrahydronaphthylethyl, and the like.

The term “heteroaryl-alkyl”, as employed herein, refers to a “heteroaryl” as defined above, appended to the parent molecular moiety through an alkyl group as defined above. The size of the alkyl can further be specified by adding the number of carbons in front of the group, e.g. heteroaryl(C₁-C₆)alkyl, heteroaryl-(C₁-C₂)alkyl. Representative examples of heteroarylalkyl include, but are not limited to, 2-(2-pyridyl)propyl, 2-benzothiophenyl-methyl, 4-(2-quinolyl)butyl, and the like.

The term “Cy-alkyl”, as employed herein, refers to a “Cy” as defined above, appended to the parent molecular moiety through an alkyl group as defined above. The size of the alkyl can further be specified by adding the number of carbons in front of the group, e.g. Cy-(C₁-C₆)alkyl, Cy-(C₁-C₃)alkyl. Representative examples of Cy-alkyl include, but are not limited to, benzyl, 1-(2-naphthyl)ethyl, 2-cyclohexylethyl, and the like.

The term “halogen”, as employed herein, refers to chlorine, bromine, fluorine or iodine.

The compounds of formula I, as well as the pharmaceutically-acceptable salts and esters thereof, are referred to below as the compounds of the invention, unless otherwise indicated.

One preferred embodiment of the compounds of formula I are those wherein Q is

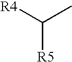

and R5 is hydrogen or (C₁-C₃)alkyl and R4 is phenyl, benzyl or phenylethyl, optionally substituted at positions 2 or 3 with one to two substituents selected from R^{a}. More preferred substituents are selected from halogen and (C₁-C₃)alkyl.

Yet another preferred embodiment of the compounds of formula I are those where E is O or NH.

Yet another preferred embodiment of the compounds of formula I are those where R3 is hydrogen and p is an integer of 1 or 2.

Yet another preferred embodiment of the compounds of formula I are those where J is hydrogen.

Yet another preferred embodiment of the compounds of formula I are those where R1 is hydrogen.

Yet another preferred embodiment of the compounds of formula I are those where j is an integer of 2 or 3.

Yet another preferred embodiment of the compounds of formula I are those where R1 is hydrogen, j is an integer of 2 or 3 and A is NH—(C═NH)NH₂ or NR6R6 with R6 independently selected from H or (C₁₁C₃)alkyl.

Yet another preferred embodiment of the compounds of formula I are those where j is 0 and A is

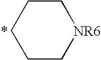

with the star denoting the point of attachment and R6 being H or (C₁-C₃)alkyl.

Yet another preferred embodiment of the compounds of formula I are those where j is 1 or 2, R1 is hydrogen and A is —NR6-(C₁-C₃)alkyl-NR6R6 or —NR6-(C₁-C₃)alkyl-NH—(C═NH)NH₂ with R6 independently selected from H or (C₁-C₃)alkyl.

Yet another preferred embodiment of the compounds of formula I are those where R2 is hydrogen or (C₁-C₆)alkyl.

Yet another preferred embodiment of the compounds of formula I are those where D is aryl, which is optionally substituted with one to three substituents selected from R^{a} and preferred substitutions R^{a} are selected from halogen, (C₁-C₆)alkyl, —NR^{b}R^{b} and —OR^{b}. Even more preferred substitutions R^{a} are halogen and (C₁-C₃)alkyl. A particularly preferred embodiment of the compounds of the invention are those in which D gives rise to compounds of formula II,

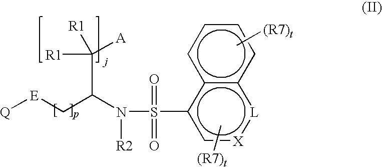

wherein A, E, Q, R1, R2, p and j are as defined above under formula I,

R1 is independently a group selected from R^{a};

X is a bond or C(R7);

L is C(R7), S or NR7;

R7 is independently selected from

1) H,

2) halogen,

3) —OR^{b},

4) (C₁-C₄)alkyl,

5) —CF₃; and

t is an integer from 0 to 2.

Yet another preferred embodiment of the compounds of formula I are those where the absolute configuration of the carbon carrying the group J is S.

The invention includes within its scope all possible stereoisomers of the compounds, including geometric isomers, e.g. Z and E isomers (cis and trans isomers), and optical isomers, e.g. diastereomers and enantiomers. Furthermore, the invention includes in its scope both the individual isomers and any mixtures thereof, e.g. racemic mixtures. The individual isomers may be obtained using the corresponding isomeric forms of the starting material or they may be separated after the preparation of the end compound according to conventional separation methods. For the separation of optical isomers, e.g. enantiomers, from the mixture thereof, the conventional resolution methods, e.g. fractional crystallisation, may be used.

Some of the compounds of the invention may also exist as tautomers, namely having different points of attachment of hydrogen. For instance, ketones can exist also in their enol form (keto-enol tautomerism). The individual tautomers as well as mixtures thereof are encompassed within the compounds of the invention.

Pharmaceutically acceptable salts, e.g. acid addition salts with both organic and inorganic acids are well known in the field of pharmaceuticals. Non-limiting examples of these salts include chlorides, bromides, sulfates, nitrates, phosphates, sulfonates, formates, tartrates, maleates, citrates, benzoates, salicylates and ascorbates. Pharmaceutically acceptable esters, when applicable, may be prepared by known methods using pharmaceutically acceptable acids that are conventional in the field of pharmaceuticals and that retain the pharmacological properties of the free form. Non-limiting examples of these esters include esters of aliphatic or aromatic alcohols, e.g. methyl, ethyl, propyl, isopropyl, butyl, isobutyl, sec-butyl and tert-butyl esters.

The pharmaceutical compositions of the compounds of the invention may be formulated in a conventional manner using one or more pharmaceutically acceptable carriers or excipients. Formulations can for instance enable an oral, buccal, topical, intranasal, parenteral (e.g. intravenous, intramuscular or subcutaneous) or rectal administration or an administration by inhalation or insufflation. Compounds of the invention may also be formulated for sustained delivery.

For oral administration, forms of suitable compositions include but are not limited to tablets, chewable tablets and capsules. These may be prepared by conventional means with pharmaceutically acceptable excipients, such as binding agents (e.g. pregelatinized maize starch), disintegrants (e.g. potato starch), fillers (e.g. lactose) or lubricants (e.g. magnesium stearate). Tablets may be coated by methods well known in the art. For oral administration, possible liquid preparations include but are not limited to solutions, syrups or suspensions, or they may exist as dry powder for constitution with water or other suitable vehicle prior to use. These liquid preparations may be prepared by conventional means with pharmaceutically acceptable agents, such as suspending agents, non-aqueous vehicles, preservatives and emulsifiers.

A possible dose of the active compounds of the invention for oral, parenteral, buccal or topical dose to the adult human is between 0.1 and 500 mg of the active compound per unit dose, which may administered, for instance, 1 to 4 times in a day.

It is well recognized that the precise dose, the route of administration and the dosing interval can be determined by those skilled in the art. It is also well recognized that these variables depend on multiple factors, including, but not restricted to, the activity of the therapeutic compound, the formulation thereof, pharmacokinetic properties (such as absorption, distribution, metabolism and excretion) of the therapeutic compound, the nature and location of the target tissue or organ and the issues connected to the state of a disease or disorder in a patient in need of treatment. Additionally, when the compounds of the invention are administered with additional pharmaceutically active ingredients, one or more pharmaceutical compositions may be used for the delivery of all the agents, which may be administered together, or at different times, as determined by those skilled in the art.

### Experimental Part

### List of Abbreviations

ACN acetonitrile

Boc tert-butyloxycarbonyl

BSA bovine serum albumin

BTHF borane-tetrahydrofuran complex

Dab 2,4-diaminobutyric acid

Dap 2,3-diaminopropionic acid

DBU 1,8-diazabicyclo[5.4.0]undec-7-ene

DCC N,N-dicyclohexylcarbodiimide

DCHA dicyclohexylamine

DCM dichloromethane

DEAD diethyl azodicarboxylate

DIC diisopropylcarbodiimide

DIPEA N,N-diisopropylethylamine

DMAP 4-dimethylaminopyridine

DMF N,N-dimethylformamide

DNP 2,4-dinitrophenyl

EDTA ethylenediamine-tetraacetic acid

ESI electrospray ionization

Fmoc 9-fluorenylmethoxycarbonyl

HEPES N-(2-hydroxyethyl)piperazine-N′-2-ethanesulfonic acid

HOBt 1-hydroxybenzotriazole

HPLC high performance liquid chromatography

IPA isopropanol

LC liquid chromatography

MS mass spectrometry

PG protecting group

PIFA Bis(trifluoroacetoxy)iodo]benzene

RP-HPLC reversed-phase high performance liquid chromatography

TEA triethylamine

TFA trifluoroacetic acid

THF tetrahydrofuran

TLC thin layer chromatography

TMOF trimethyl orthoformate

TMS tetramethylsilane

TRIS tris(hydroxymethyl)aminomethane

Z benzyloxycarbonyl

Compounds of the invention can be prepared using the following general synthetic schemes.

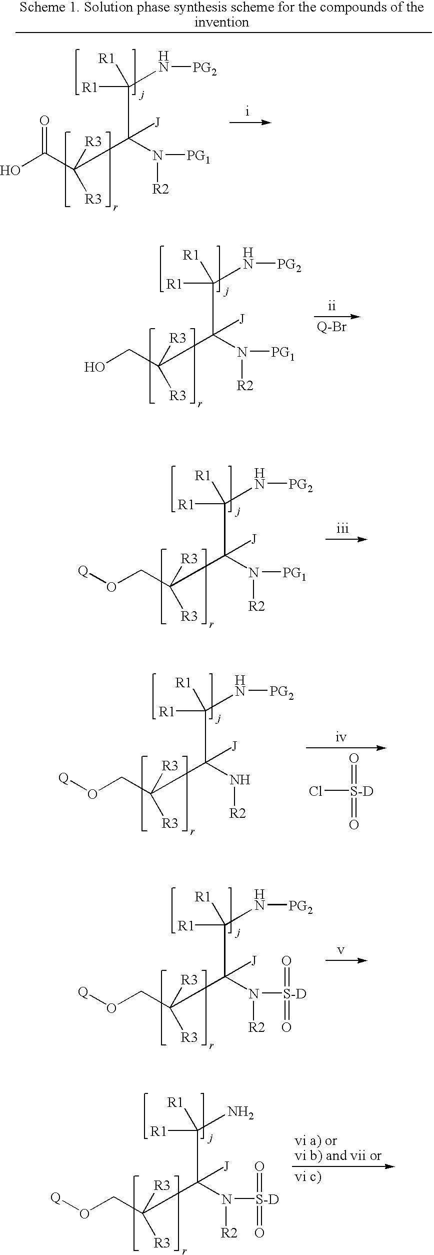

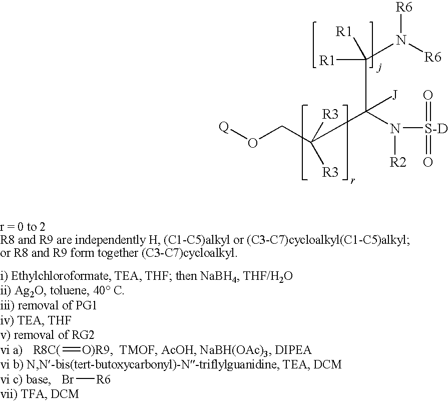

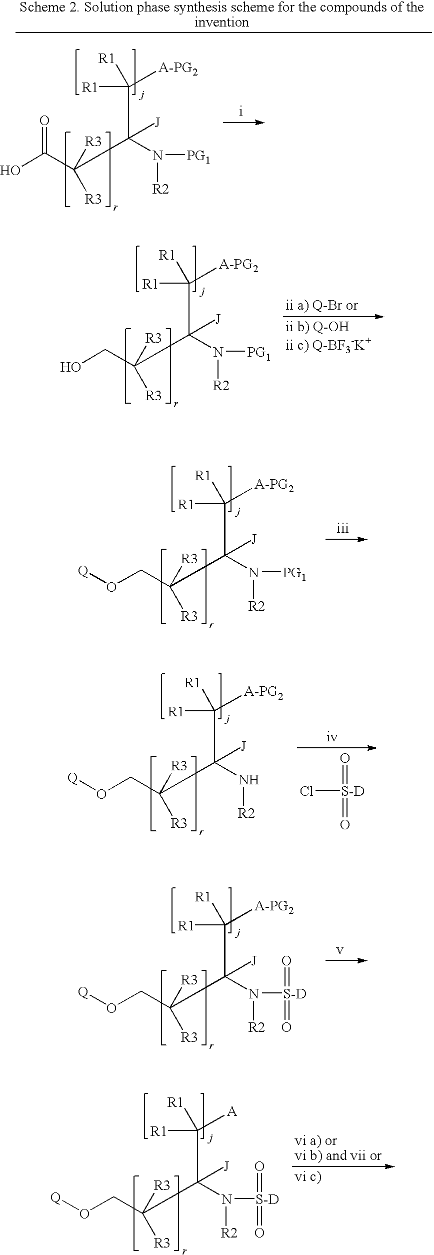

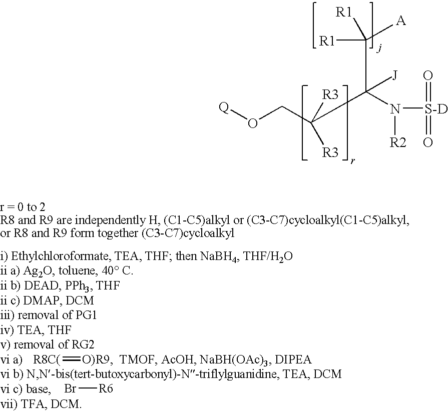

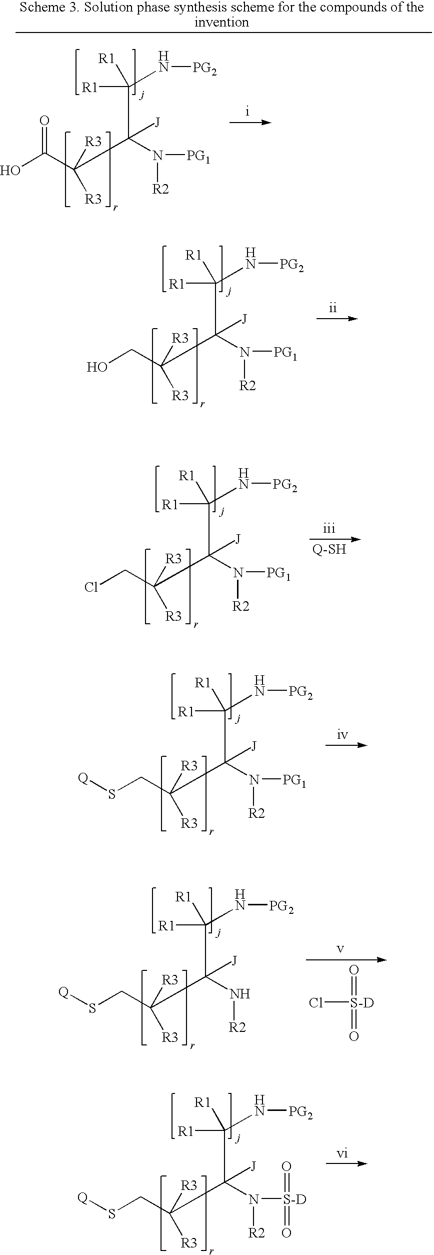

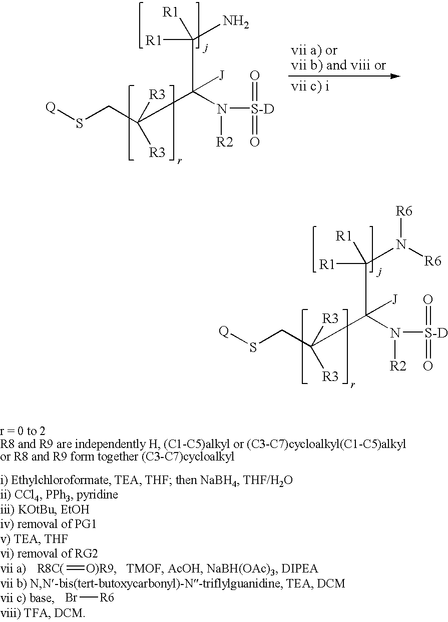

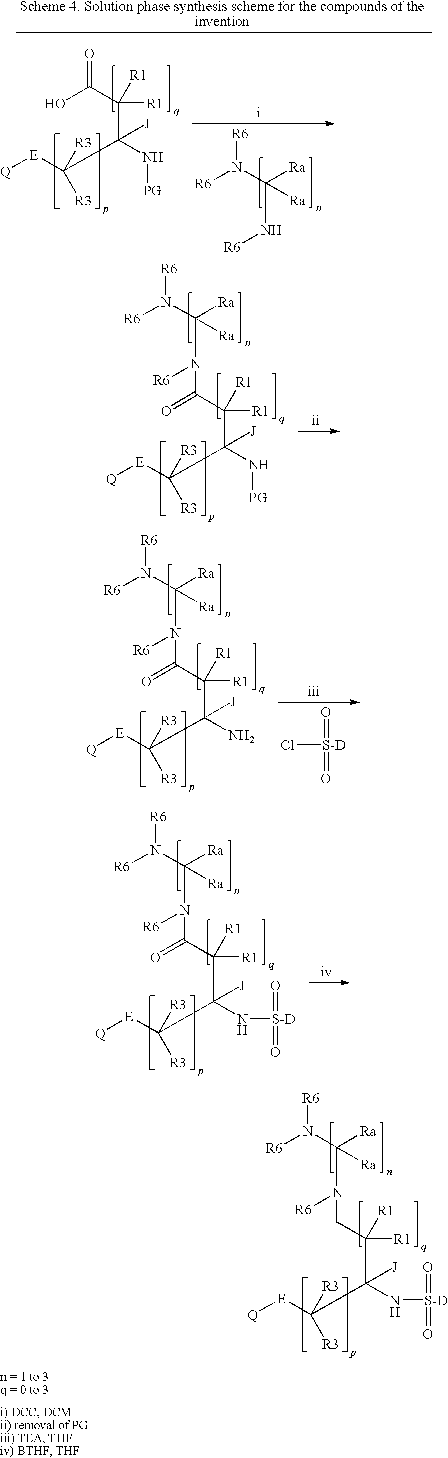

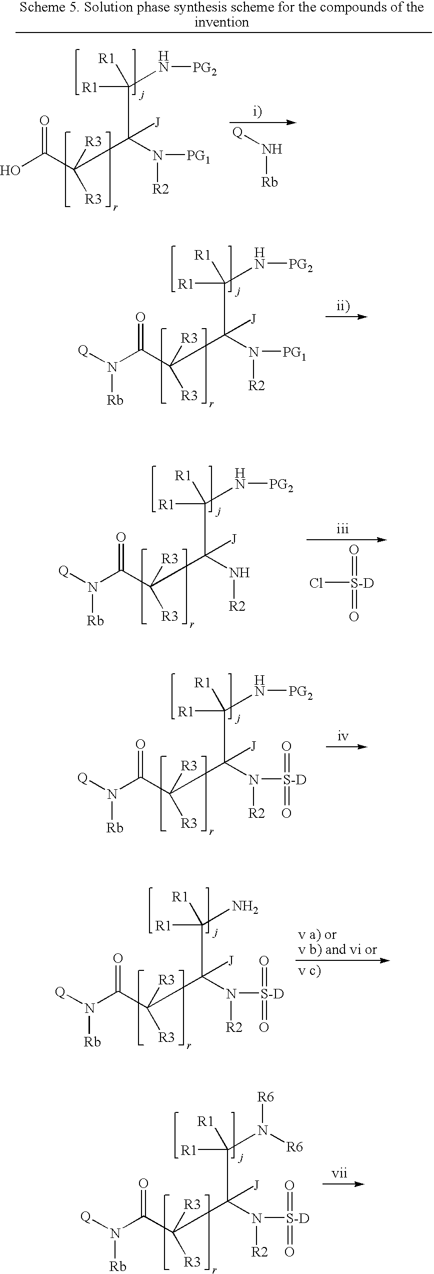

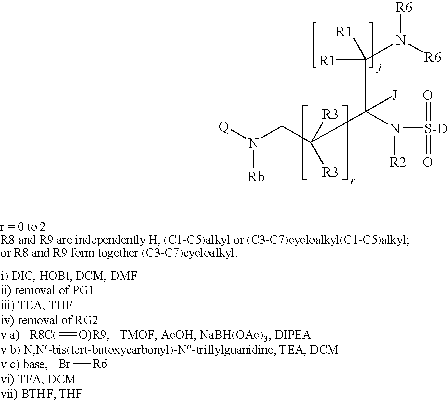

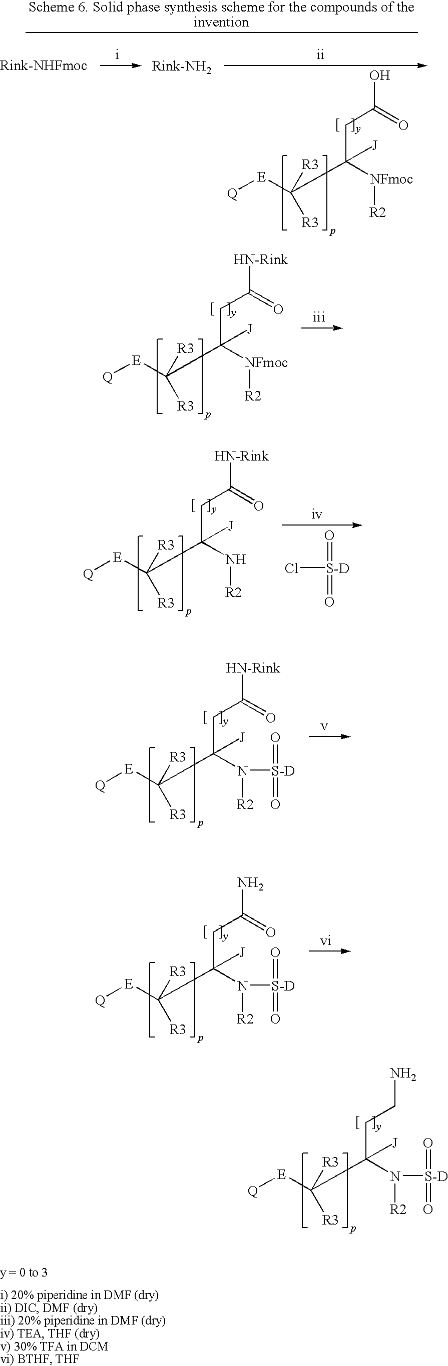

It's evident for a person skilled in the art that these general schemes can be further modified, for example by using different protecting groups (e.g. those described in T. W. Greene and P. G. M. Wuts, “Protective Groups in Organic Synthesis”, 2^{nd} ed. Wiley, 1991, New York, US), or by adding or removing steps in between or after the described steps, which enables additional synthetic modifications including, but not limited to, the examples given.

### Starting Materials

The Rink resin was obtained from Advanced ChemTech, UK. Amino acids were purchased either from Advanced ChemTech, UK, or Nova-biochem, Switzerland, unless otherwise specified. Acetic anhydride, benzyl bromide, benzyl chloroformate, BTHF, DIC, ethyl chloroformate, HOBt, piperidine, silver(I) oxide, sodium triacetoxyborohydride, TFA, alpha-toluenethiol were products of Acros Organics, Belgium. DIPEA was from Fluka AG, Germany. All other reagents or solvents were purchased from Aldrich or Merck, Germany, if not otherwise specified. The reagents were used as such and solvents were purified and dried according to the methods described in W. L. F. Armareggo and D. D. Perrin, “Purification of Laboratory Chemicals”, 4^{th} ed. Butterworth-Heinemann, 1996, Bath, Great Britain.

### General Description of MS Analysis

The molecular weight of compound was determined with a Micromass Micro triple quadrupole mass spectrometer. Essential MS parameters were: cone voltage 30 V, capillary voltage 3.5 kV, low mass resolution on MS1 15, high mass resolution on MS1 15, ion energy on MS1 1.0, source temperature 110° C., desolvation temperature 250° C. and desolvation gas flow 700 l/h. Samples were introduced via a Waters Alliance 2695 HPLC instrument. The flow rate of 0.3 ml/min was formed of 10% water and 90% MeOH eluent (containing 0.01% HCOOH). Sample volumes of 10 μl were injected through a Waters Symmetry Shield 2.1×10 mm C₁₈ precolumn.

### General Description of LC-MS Analysis

For LC-MS analysis the gradient started with 100% water (containing 0.01% HCOOH) (A) which changed linearly over ten minutes to 100% ACN (containing 0.01% HCOOH) (B). In addition, a Waters Symmetry Shield 2.1×50 mm C₁₈ column with a corresponding precolumn was flushed for two minutes with B. The flow rate used was 0.4 ml/min and sample volumes of 10 μl were injected. Some essential MS parameters were increased compared to the standard MS analysis procedure: the desolvation temperature was changed to 350° C. and the desolvation gas flow to 900 l/h. The UV chromatogram was recorded with a Waters 996 diode array detector.

### General Description of NMR Analysis

NMR spectra were recorded on a Bruker DMX 500 spectrometer operating at 500.13 MHz for ¹H. CD₃OD was used as the solvent and TMS as the internal standard.

### General Description of RP-HPLC Purification

Semi-preparative RP-HPLC purifications were done with a Waters 616 pump connected to a Waters 600 controller unit. The instrument was equipped with a Waters 2487 UV detector and a Waters fraction collector. An Xterra Prep C₁₈ RP 10×150 mm column with a 7.8×20 mm precolumn was used for purifications. The flow rate was 6.6 ml/min and the detection wavelength 254 nm. The gradient started with water (containing 0.3% HCOOH) (A) and changed linearly to ACN (containing 0.3% HCOOH) (B) over a period of ten minutes. In addition, the column was flushed with B for two minutes. The fraction collector was programmed to collect 30 s fractions, which were analysed by MS.

### General Description of Automated RP-LC Purification

Automated RP-LC purifications were done with a Biotage Flash Master II flash chromatography purification system using Supelco discovery DSC-18 columns (2-10 g). The flow rate was 5-15 ml/min, depending on the column size. The detection wavelength was 254 nm. The gradient started from water (100%) and changed to MeCN or MeOH (100%) over a period of 6-10 minutes. The fractions were collected with the aid of the a program-controlled fractions collector and analysed by LC-MS.

### General Description of Silica Gel Chromatographic Purifications

Silica gel purchased from Merck (grade 60, mesh 0.063-0.200 mm) was used in column chromatography purifications. The eluent was 1 to 25% MeOH in DCM, if not otherwise specified.

### General Description of Preparative TLC Chromatographic Purifications

Preparative TLC plates purchased from Merck (grade 60, F₂₅₄, 2 mm) were used. The eluent was 30% MeOH in DCM.

### Naming of the Compounds

As a sulfonamide group is a common feature in all compounds of the invention, the compounds are named as sulfonic acid amides.

### Example 1

### Synthesis of (S)-4-methylnaphthalene-1-sulfonic acid (4-amino-1-benzylsulfanylmethylbutyl)amide (compound 1)

### Step I

Boc-L-Ornithinol(Z) (510 mg, 352.43 g/mol, 1.45 mmol, 1 eq, Glycoteam, Germany) was dissolved in pyridine (2 ml, dry) under argon atmosphere. Triphenylphosphine (0.949 g, 262.29 g/mol, 3.62 mmol, 2.5 eq, dissolved in 2 ml of dry pyridine), tetrachloromethane (420 μl, 153.82 g/mol, 1.59 g/cm3, 4.34 mmol, 3 eq) and a small amount of molecular sieves were added to the reaction mixture. After reacting overnight, the mixture was filtered and evaporated from toluene. The thus obtained crude product was purified by chromatography to obtain 294 mg (55% yield) of (S)-5-chloro-4-N-Boc-1-N′-Z-pentane-1,4-diamine in pure form.

### Step II

Alpha-toluenethiol (158 μl, 124.21 g/mol, 1.058 g/cm3, 1.35 mmol, 1.7 eq) was added to a solution of KOtBu (133 mg, 112.21 g/mol, 1.19 mmol, 1.5 eq) in EtOH (1.5 ml, dry) under argon atmosphere. After 1 h of stirring, (S)-5-chloro-4-N-Boc-1-N′-Z-pentane-1,4-diamine (294 mg, 370.88 g/mol, 0.793 mmol, 1 eq) was dissolved in EtOH (3.5 ml) and added to the reaction mixture. The reaction mixture was allowed to react overnight before it was filtered and the filtrate was evaporated. The residue was dissolved in DCM and washed with water and brine. The organic phase was dried over Na₂SO₄, evaporated and purified by chromatography to obtain 248 mg (68% yield) of (S)-5-benzylsulfanyl-4-N-Boc-1-N′-Z-pentane-1,4-diamine in pure form.

### Step III

The Boc protection was removed by dissolving (S)-5-benzylsulfanyl-4-N-Boc-1-N′-Z-pentane-1,4-diamine (248 mg, 458.62 g/mol, 0.54 mmol) in 10 ml DCM containing 25% TFA. After 45 min of stirring, the solvent was evaporated and the residue was twice evaporated from water to quantitatively obtain (S)-5-benzylsulfanyl-1-N-Z-pentane-1,4-diamine in form of its trifluoroacetic acid salt.

### Step IV

(S)-5-benzylsulfanyl-1-N-Z-pentane-1,4-diamine trifluoroacetic acid salt (0.54 mmol, 1 eq) was dissolved in dry THF (6 ml). TEA (300 μL, 2.15 mmol, 4 eq) and 4-methyl-1-naphtalene sulfonyl chloride (285 mg, 270.71 g/mol, 1.18 mmol, 2.2 eq, Maybridge, UK) were added and the resulting mixture was stirred overnight at room temperature. The reaction mixture was filtered and the filtrate was evaporated. The residue was purified by chromatography to obtain 262 mg (87% yield) of (S)-4-methylnaphthalene-1-sulfonic acid [4-(N-Z-amino)-1-benzylsulfanylmethylbutyl]amide.

### Step V

The Z-protection was removed by dissolving (S)-4-methyl-naphthalene-1-sulfonic acid [4-(N-Z-amino)-1-benzylsulfanylmethylbutyl]amide (130 mg, 562.75 g/mol, 0.23 mmol, 1 eq) in ACN (2 ml), followed by the addition of chlorotrimethylsilane (118 μl, 108.64 g/mol, 0.85 g/cm³, 0.92 mmol, 4 eq) and sodium iodide (138 mg, 149.89 g/mol, 0.92 mmol, 4 eq). After having reacted overnight, the mixture was evaporated and the residue was taken up in DCM (50 ml) and washed with aq. 10% Na₂S₂O₃ solution (3×50 ml). The organic phase was dried over Na₂SO₄, and the reaction product was purified by chromatography to obtain 44 mg (40% yield) of the title compound in pure form.

MS-ESI⁺ (m/z): 429

¹H NMR (500 MHz, CD₃OD; δ, ppm): 8.72 (m, 1H), 8.21 (m, 1H), 8.13 (m, 1H), 7.73-7.69 (m, 2H), 7.46 (m, 1H), 7.17-7.14 (m, 3H), 6.90 (m, 2H), 3.25-3.20 (m, 1H), 3.15 (m, 2H), 2.85-2.73 (m, 2H), 2.77 (s, 3H), 2.14-2.06 (m, 2H), 1.73-1.66 (m, 2H), 1.63-1.55 (m, 1H), 1.42-1.36 (m, 1H).

### Example 2

### Synthesis of (S)-4-methylnaphthalene-1-sulfonic acid (3-amino-1-benzyl-aminomethylpropyl)amide (compound 2)

### Step I

Fmoc-L-Dab(Boc)-OH (1.00 g, 440.50 g/mol, 2.27 mmol, 1 eq), DIC (355 μl, 126.20 g/mol, 0.806 g/cm3, 2.27 mmol, 1 eq) and HOBt (308 mg, 135.12 g/mol, 2.27 mmol, 1 eq) were dissolved in DMF/DCM (1/1, 10 ml, dry). After 5 minutes of stirring, benzylamine (248 μl, 107.16 g/mol, 0.981 g/cm³, 2.27 mmol, 1 eq, Acros) was added to the reaction mixture and the stirring was continued overnight at 35° C. The reaction mixture was then evaporated and the residue purified by chromatoraphy. In this manner 1.17 g (98% yield) of (S)—N-benzyl-4-(N′-Boc-amino)-2-(N″-Fmoc-amino)butyramide were obtained.

### Step II

The Fmoc protection was removed by dissolving the (S)—N-benzyl-4-(N′-Boc-amino)-2-(N″-Fmoc-amino)butyramide (1.12 g, 529.64 g/mol, 2.1 mmol) in 10 ml DMF containing 20 vol-% piperidine. After 1.5 h of stirring, the solvent and excess piperidine were evaporated. The thus obtained crude (S)-2-amino-4-(N-Boc-amino)-N′-benzylbutyramide was used without further purification for step III.

### Step III

(S)-2-amino-4-(N-Boc-amino)-N′-benzylbutyramide (2.1 mml, 1 eq) was dissolved in THF (7 ml, dry) and 4-methyl-1-naphthalenesulfonyl chloride (761 mg, 240.71 g/mol, 3.15 mmol, 1.5 eq) and TEA (440 μl, 3.15 mmol, 1.5 eq) were added to the reaction mixture. After overnight stirring at room temperature, the solvent was evaporated and the residue purified by chromatography to give 860 mg (80% yield) of (S)-4-methylnaphthalene-1-sulfonic acid [3-(N-Boc-amino)-1-benzylcarbamoylpropyl]amide in pure form.

### Step IV

The Boc protection was removed by dissolving the (S)-4-methylnaphthalene-1-sulfonic acid [3-(N-Boc-amino)-1-benzylcarbamoylpropyl]amide (0.85 g, 511.65 g/mol, 1.7 mmol) in 10 ml DCM containing 25% TFA. After 1 h of stirring, the solvent was evaporated and the residue purified by chromatography to obtain 0.80 g (89% yield) of (S)-4-methylnaphthalene-1-sulfonic acid (3-amino-1-benzylcarbamoylpropyl)amide in the form of its trifluoroacetic acid salt.

### Step V

The (S)-4-Methylnaphthalene-1-sulfonic acid (3-amino-1-benzylcarbamoylpropyl)amide trifluoroacetic acid salt (101 mg, 539.58 g/mol, 0.19 mmol, 1 eq) was dissolved in THF (1.5 ml, dry) under argon atmosphere. BTHF (1.9 ml, 1.0 M, 1.9 mmol, 10 eq, Acros) was added dropwise by syringe to the reaction mixture. After a 5 h reaction time at 60° C., the reaction mixture was cooled to room temperature and quenched by dropwise adding MeOH (0.5 ml). The solvents were evaporated, the residue was mixed with water (1 ml) and HCl (1 ml, 6 M) and stirred at 50° C. for 1.5 h. The acidic water phase was then washed with DCM and made alkaline by adding NaOH (5 M) before the product was extracted with EtOAc. The organic phase was dried over Na₂SO₄ and evaporated. The residue was purified by semi-preparative RP-HPLC-chromatography to obtain 14 mg (19% yield) of the title compound in pure form.

MS-ESI⁺ (m/z): 398

¹H NMR (500 MHz, CD₃OD; δ, ppm): 8.68 (m, 1H), 8.40 (s, br, 1H), 8.20 (m, 1H), 8.17 (d, 1H), 7.74-7.68 (m, 2H), 7.45 (m, 1H), 7.30-7.27 (m, 3H), 7.07 (m, 2H), 3.62 (m, 2H), 3.47 (m, 1H), 2.76 (s, 3H), 2.71 (m, 1H), 2.65-2.55 (m, 3H), 1.82-1.76 (m, 1H), 1.70-1.64 (m, 1H)

### Example 3

### Synthesis of (S)-4-methylnaphthalene-1-sulfonic acid (3-amino-1-benzyl-oxymethylpropyl)amide (compound 3)

### Step I

Fmoc-L-Dab(Boc)-OH (402 mg, 440.50 g/mol 0.91 mmol, 1 eq) was dissolved in dry THF (10 ml). TEA (132 μl, 0.95 mmol, 1 eq) was added and the resulting mixture was cooled to −5° C. in an ice/salt bath. Ethyl chloroformate (91 μl, 108.53 g/mol, 1.135 g/cm³, 0.95 mmol, 1 eq) was added dropwise to the mixture. After a 30 min reaction time the formed precipitate was filtered off. The filtrate was added dropwise to a freshly prepared and cooled (−5° C.) solution of sodium borohydride (43.7 mg, 37.83 g/mol, 1.16 mmol, 1.3 eq) in 2 ml of H₂O/THF. The resulting mixture was stirred at −5° C. for 1.5 h and then allowed to warm up to room temperature. The solvent was evaporated and the residue dissolved in EtOAc (30 ml) before it was washed successively with a 10% citric acid solution, a 5% NaHCO₃ solution, water and brine. The organic phase was dried over Na₂SO₄. Filtration and evaporation gave a crude product which was purified by chromatography to obtain 282 mg (72% yield) of (S)-4-(N-Boc-amino)-2-(N′-Fmoc-amino)butan-1-ol.

### Step II

(S)-4-(N-Boc-amino)-2-(N′-Fmoc-amino)butan-1-ol (282 mg, 426.52 g/mol, 0.66 mmol, 1.0 eq) was dissolved in toluene (10 ml, dry) under argon atmosphere. Silver(I) oxide (461 mg, 231.73 g/mol, 1.99 mmol, 3.0 eq) was added and the resulting mixture was cooled to 0° C. in an ice bath. Benzyl bromide (197 μl, 171.04 g/mol, 1.438 g/cm³, 1.65 mmol, 2.5 eq) was dissolved in toluene (2 ml) and the solution was added dropwise to the cooled reaction mixture. The reaction mixture was stirred at 0° C. for 1 hour before it was allowed to warm to room temperature. After continuing the stirring overnight at 45° C. there was still starting material left. Therefore silver(I) oxide (154 mg, 1 eq) and benzyl bromide (79 μl, 1 eq) were added and the stirring was continued for another night before the reaction mixture was filtered and the filtrate evaporated. The residue was purified by chromatography to obtain 210 mg (62% yield) of (S)-4-benzyloxy-3-N-Boc-1-N′-Fmoc-butane-1,3-diamine.

### Step III

The Fmoc protection was removed from (S)-4-benzyloxy-3-N-Boc-1-N′-Fmoc-butane-1,3-diamine (210 mg, 516.64 g/mol, 0.406 mmol) as described in Example 2, Step II. The crude product was purified by chromatography to obtain 72 mg (60% yield) of (S)-4-benzyloxy-3-N-Boc-butane-1,3-diamine in pure form.

### Step IV

(S)-4-benzyloxy-3-N-Boc-butane-1,3-diamine (36 mg, 0.12 mmol, 1 eq) was dissolved in THF (1.7 ml, dry). TEA (44 μl, 0.32 mmol, 2.6 eq) and 4-methyl-1-naphtalene sulfonyl chloride (47 mg, 0.18 mmol, 1.4 eq) were added and the resulting mixture was stirred overnight at room temperature. The reaction mixture was evaporated and the residue was purified by chromatography to give 50 mg (82% yield) of (S)-4-methylnaphthalene-1-sulfonic acid [3-(N-Boc-amino)-1-benzyloxymethylpropyl]amide.

### Step V

The Boc protection was removed by dissolving (S)-4-methyl-naphthalene-1-sulfonic acid [3-(N-Boc-amino)-1-benzyloxymethylpropyl]amide (50 mg, 498.65 g/mol, 0.10 mmol) in 5 ml DCM containing 20 vol % TFA. The reaction mixture was stirred at room temperature for 1.5 h before it was diluted with DCM (25 ml), washed successively with a 5% NaHCO₃ solution and brine and dried over anhydrous Na₂SO₄. Filtration and evaporation of the solvent gave the title compound in quantitative yield (40 mg).

MS-ESI⁺ (m/z): 399

¹HNMR (500 MHz, CD₃OD; δ, ppm): 8.72 (m, 1H), 8.16 (m, 1H), 8.13 (d, 1H), 7.66 (m, 2H), 7.40 (m, 1H), 7.20 (m, 3H), 6.96 (m, 2H), 3.96 (m, 2H), 3.38 (m, 1H), 3.05 (dd, 1H, J=4.3 Hz, J=9.6 Hz), 2.92 (dd, 1H, J=5.6 Hz, J=9.6 Hz), 2.75 (m, 3H), 2.59 (m, 2H), 1.59 (m, 2H).

### Example 4

### Synthesis of (S)-4-methylnaphthalene-1-sulfonic acid (1-benzyloxymethyl-3-guanidinylpropyl)amide (compound 4)

### Step I

(S)-4-Methylnaphthalene-1-sulfonic acid (3-amino-1-benzyloxy-methylpropyl)amide (32 mg, 398.53 g/mol, 80 μmol, 1 eq, Example 3) was dissolved in DCM (3 ml) and TEA (41 μl, 0.30 mmol, 3.7 eq) before N,N′-bis(Boc)-N″-triflylquanidine (59 mg, 391.37 g/mol, 0.15 mmol, 1.9 eq) were added. After 2.5 h of stirring at room temperature, the solvent was evaporated and the reaction product purified by chromatography to give 51 mg (99% yield) of (S)-4-methylnaphthalene-1-sulfonic acid [1-benzyloxymethyl-3-(N,N′-bis(Boc)guanidinylpropyl]amide.

### Step II

The Boc protections were removed by dissolving (S)-4-methylnaphthalene-1-sulfonic acid [1-benzyloxymethyl-3-(N,N′-bis(tertbutoxycarbonyl)guanidinylpropyl]amide (51 mg, 640.80 g/mol, 79 mmol) in 6 ml DCM containing 20% TFA. The resulting mixture was stirred at room temperature for 4 h before it was diluted with DCM and sequentially washed with a 5% NaHCO₃ solution and brine. The organic phase was dried over Na₂SO₄ and filtered before the filtrate was evaporated to give 26 mg (59% yield) of the title compound.

MS-ESI⁺ (m/z): 441

¹HNMR (500 MHz, CD₃OD; δ, ppm): 8.71 (m, 1H), 8.18 (m, 1H), 8.13 (d, 1H, J=7.5 Hz), 7.67 (m, 2H), 7.41 (d, 1H, J=7.5 Hz), 7.20 (m, 3H), 6.94 (m, 2H), 3.94 (m, 2H), 3.37 (m, 1H), 3.19 (m, 2H), 3.00 (dd, 1H, J=4.4 Hz, J=9.4 Hz), 2.9 (dd, 1H, J=5.6 Hz, J=9.4 Hz), 2.76 (m, 3H), 1.79 (m, 1H), 1.66 (m, 1H)

### Example 5

### Synthesis of 4-methylnaphthalene-1-sulfonic acid (2-benzyloxy-1-piperidin-4-yl-ethyl)amide (compound 5)

### Step I

N-Fmoc-(1-Boc-piperidin-4-yl)-D,L-glycine (1.09 g, 480.57 g/mol, 2.27 mmol, 1 eq), TEA (331 μl, 2.38 mmol, 1.05 eq), ethyl chloroformate (229 μl, 2.39 mmol, 1.05 eq) and sodium borohydride (98.7 mg, 2.60 mmol, 1.15 eq) were allowed to react according to the procedure described in Example 3, step I. In this manner 586 mg (55% yield) of 2-(N-Boc-piperidin-4-yl)-2-(N′-Fmoc-amino)ethanol were obtained.

### Step II

2-(N-Boc-piperidin-4-yl)-2-(N′-Fmoc-amino)ethanol (180 mg, 466.58 g/mol, 0.39 mmol, 1 eq) was treated with benzyl bromide (280 μl, 2.35 mmol, 6.1 eq) and silver(I) oxide (54 mg, 2.33 mmol, 6 eq) in toluene according to the procedure described in Example 3, step II. After chromatographic purification, 110 mg (51% yield) of 2-benzyloxy-1-(N-Boc-piperidin-4-yl)-N′-Fmoc-ethylamine was obtained in pure form.

### Step III

The Fmoc protection was removed from 2-benzyloxy-1-(N-Boc-piperidin-4-yl)-N′-Fmoc-ethylamine (115.8 mg, 556.71 g/mol, 28 μmol) according to Example 3, step III. Chromatographic purification yielded 25 mg (36% yield) of 2-benzyloxy-1-(N-Boc-piperidin-4-yl)ethylamine.

### Step IV

2-Benzyloxy-1-(N-Boc-piperidin-4-yl)ethylamine (25 mg, 334.46 g/mol, 75 μmol, 1 eq) was sulfonylated with 4-methyl-1-naphtalene sulfonyl chloride (110 mg, 0.46 mmol, 6.1 eq) according to Example 3, step IV. After chromatographic purification, 34 mg (84% yield) of 4-methylnaphthalene-1-sulfonic acid [2-benzyloxy-1-(N-Boc-piperidin-4-yl)ethyl]amide were obtained in pure form.

### Step V

The Boc protection was removed by treating 4-methylnaphthalene-1-sulfonic acid [2-benzyloxy-1-(N-Boc-piperidin-4-yl)ethyl]amide (34 mg, 538.71 g/mol, 63 μmol) with TFA according to the procedure described in Example 3, step V. Chromatographic purification yielded 21 mg (74% yield) the title compound in pure form.

MS-ESI⁺ (m/z): 439

¹HNMR (500 MHz, CD₃OD; δ, ppm): 8.64 (m, 1H), 8.17 (m, 1H), 8.14 (d, 1H, J=7.5 Hz), 7.66 (m, 2H), 7.43 (d, 1H, J=7.5 Hz), 7.28 (m, 3H), 7.20 (m, 2H), 4.32 (s, 2H), 3.87 (m, 1H), 3.47 (m, 1H), 3.45 (m, 1H), 3.42 (m, 1H), 3.05 (m, 2H), 2.76 (s, 3H), 2.60 (m, 2H), 1.73 (m, 2H), 1.61 (m, 2H)

### Example 6

### Synthesis of (S)-4-methylnaphthalene-1-sulfonic acid [1-aminomethyl-2-(naphthalen-2-ylmethoxy)ethyl]amide (compound 6)

### Step I

Boc-L-Dap-OH (817 mg, 204.23 gμmol, 4.0 mmol, 1 eq) was dissolved in MeOH (6 ml, dry) under argon atmosphere. The reaction mixture was cooled to 0° C. and TEA (1.11 ml, 8.0 mmol, 2 eq) was added. After 10 min benzyl chloroformate (570 μl, 170.6 g/mol, 1.2 g/cm³, 4.0 mmol, 1 eq) was added dropwise and the reaction mixture was allowed to warm up to room temperature. After allowing to react overnight, the reaction mixture was evaporated. The residue was taken up in water, made acidic by adding HCl (1 M) and extracted with EtOAc. The organic phase was dried over Na₂SO₄ and the solvent evaporated. The residue was purified by chromatography to give 736 mg (54% yield) of (S)-2-(N-Boc-amino)-3-(N′-Z-amino)propionic acid in pure form.

### Step II

(S)-2-(N-Boc-amino)-3-(N′-Z-amino)propionic acid (736 mg, 338.36 g/mol, 2.18 mmol, 1 eq) was dissolved in THF (6.5 ml, dry) and cooled to −10° C. 4-Methylmorpholine (240 μl, 101.15 g/mol, 0.918 g/cm³, 2.18 mmol, 1 eq) was added to the reaction mixture, followed by the addition of methyl chloroformate (180 μl, 94.5 g/mol, 1.223 g/cm³, 2.33 mmol, 1.07 eq). After 10 min, a freshly prepared solution of sodium borohydride (249 mg, 6.58 mmol, 3 eq) in water (2 ml) was added dropwise to the reaction mixture at −15° C. After another 10 min of reaction time, the mixture was poured into water (ca. 25 ml) and stirred for a further 10 min at room temperature. The water phase was extracted with EtOAc (3×) before the combined organic phases were washed successively with HCl (1 M), water, sat. aq. NaHCO₃ and brine. The organic phase was then dried over Na₂SO₄ and evaporated to give 0.661 mg (94% yield) of (S)-2-(N-Boc-amino)-3-(N′-Z-amino)propan-1-ol.

### Step III

The Boc protection was removed by dissolving (S)-2-(N-Boc-amino)-3-(N-Z-amino)propan-1-ol (661 mg, 324.38 g/mol, 2.04 mmol) in DCM containing 25% TFA and allowing the solution to stand for 1.5 h. After evaporation of the solvent the obtained crude (S)-2-amino-3-(N-Z-amino)propan-1-ol trifluoroacetic acid salt was used without further purification in the next reaction step.

### Step IV

(S)-2-Amino-3-(N-Z-amino)propan-1-ol trifluoroacetic acid salt (2.04 mmol, 1 eq) was sulfonylated with 4-methyl-1-naphtalene sulfonyl chloride (789 mg, 3.28 mmol, 1.6 eq) according to Example 3, step IV, to obtain 289 mg (33% yield) of (S)-4-methylnaphthalene-1-sulfonic acid [1-(N-Z-amino)methyl-2-hydroxyethyl]amide.

### Step V

(S)-4-Methylnaphthalene-1-sulfonic acid [1-(N-Z-amino)methyl-2-hydroxyethyl]amide (143 mg, 428.51 g/mol, 0.33 mmol, 1 eq) was treated with 2-(bromomethyl)naphtalene (215 mg, 221.1 g/mol, 0.97 mmol, 2.9 eq) and silver(I) oxide (206 mg, 0.89 mmol, 2.66 eq) in toluene according to the procedure described in Example 3, step II, except that the reaction time was 4 d. The thus obtained crude (S)-4-methylnaphthalene-1-sulfonic acid [1-(N-Z-amino)methyl-2-(naphthalen-2-ylmethoxy)ethyl]amide was used without further purification in the next step.

### Step IV

The Z protection was removed by dissolving crude (S)-4-methylnaphthalene-1-sulfonic acid [1-(N-Z-amino)methyl-2-(naphthalen-2-ylmethoxy)ethyl]amide (0.33 mmol) in MeOH (5 ml) and adding 10% Pd/C (128 mg). The resulted reaction mixture was hydrogenated at normal pressure. After allowing to react overnight, the reaction was stopped and the catalyst was filtered off. The filtrate was evaporated and purified by chromatography to give 17 mg (12% yield) of the title compound in pure form.

MS-ESI⁺ (m/z): 435

¹HNMR (500 MHz, CD₃OD; δ, ppm): 8.68 (d, 1H, J=8.6 Hz), 8.13 (d, 1H, J=7.6 Hz), 7.95 (d, 1H, J=8.6 Hz), 7.82 (m, 1H), 7.75 (m, 1H), 7.72 (d, 1H, J=8.6 Hz), 7.62 (m, 1H), 7.48 (m, 3H), 7.40 (s, 1H), 7.34 (d, 1H, J=7.6 Hz), 7.08 (m, 1H), 3.50 (s, 2H), 3.45 (m, 1H), 3.34 (m, 2H), 2.68 (m, 1H) 2.60 (s, 3H), 2.54 (m, 1H)

### Example 7

### Synthesis of (S)—N-(4-amino-1-benzyloxymethylbutyl)-2,3,4,5,6-pentamethyl-benzenesulfonamide (compound 7)

### Step I

Fmoc-L-(Orn(Boc)-OH (500 mg, 454.5 g/mol, 1.1 mmol, 1 eq), TEA (168 μl, 1.21 mmol, 1.1 eq), ethylchloroformate (116 μl, 1.21 mmol, 1.1 eq) and sodium borohydride (62 mg, 1.65 mmol, 1.5 eq) were allowed to react according to the procedure described in Example 3, step I. After a chromatographic purification 328 mg (66% yield) of (S)-5-(N-Boc-amino)-2-(N′-Fmoc-amino)pentan-1-ol were obtained.

### Step II

(S)-5-(N-Boc-amino)-2-(N′-Fmoc-amino)pentan-1-ol (200 mg, 440.54 g/mol, 0.45 mmol, 1 eq) was treated in toluene with benzyl bromide (240 μl, 2.0 mmol, 4.5 eq) and silver(I) oxide (210 mg, 0.91 mmol, 2 eq) according to the procedure described in Example 3, step II, except that the reaction time was 4d. In this manner 48 mg (20% yield) of (S)-5-benzyloxy-N-1-Boc-N′-4-Fmoc-pentane-1,4-diamine were obtained in pure form.

### Step III

The Fmoc protection was removed by treating (S)-5-benzyloxy-N-1-Boc-N′-4-Fmoc-pentane-1,4-diamine (48 mg, 530.67 g/mol, 91 μmol) with piperidine according to Example 3, step III. Purification by chromatography gave (S)-5-benzyloxy-N-1-Boc-pentane-1,4-diamine 16 mg (57%).

### Step IV

(S)-5-benzyloxy-N-1-Boc-pentane-1,4-diamine (16 mg, 308.42 g/mol, 52 μmol, 1 eq) was sulfonylated according to Example 3, step IV, with the exception that pentamethylbenzenesulfonyl chloride (19 mg, 78 μmol, 1.5 eq) instead of 4-methyl-1-naphtalene sulfonyl was used. After chromatographic purification 13 mg (48% yield) of (S)—N-[4-(N′-Boc-amino)-1-benzyloxymethylbutyl]-2,3,4,5,6-pentamethylbenzenesulfonamide were obtained.

### Step V

The Boc protection was removed by treating (S)—N-[4-(N′-Boc-amino)-1-benzyloxymethylbutyl]-2,3,4,5,6-pentamethylbenzenesulfonamide (13 mg, 518.72 g/mol, 25 mmol) with TFA according to the procedure described in Example 3, step V, to yield 8.4 mg (80% yield) of the title compound.

MS-ESI⁺ (m/z): 419

¹HNMR (500 MHz, CD₃OD; δ, ppm): 7.25 (m, 3H), 7.15 (m, 2H), 4.26 (s, 2H), 4.22 (m, 1H), 3.34 (m, 1H), 3.28 (m, 1H), 3.17 (m, 1H), 2.58 (m, 1H), 2.55 (s, 6H), 2.27 (s, 3H), 2.21 (s, 6H), 1.48 (m, 4H)

### Example 8

### Synthesis of (S)-2-naphthalen-1-yl-ethanesulfonic acid (4-amino-1-benzyloxymethylbutyl)amide (compound 8)

### Step I

(S)-5-Benzyloxy-N-1-Boc-pentane-1,4-diamine (42 mg, 0.14 mmol, 1 eq), obtained according to the procedure described in Example 7, steps I-III, was sulfonylated according to Example 3, step IV, with the exception that 2-(1-naphtyl)ethanesulfonyl chloride (46 mg, 254.74 g/mol, 0.18 mmol, 1.3 eq, ASDI) was used instead of 4-methyl-1-naphtalenesulfonyl chloride. In this manner 11 mg (15% yield) of (S)-2-naphthalen-1-yl-ethanesulfonic acid [4-(N-Boc-amino)-1-benzyloxymethylbutyl]amide were obtained.

### Step II

(S)-2-naphthalen-1-yl-ethanesulfonic acid [4-(N-Boc-amino)-1-benzyloxymethylbutyl]amide (11 mg, 526.70 g/mol, 20 μmol) was treated with TFA according to the procedure described in Example 4, step II, except that the reaction time was 45 min. The crude product was purified twice by chromatography to give 5.1 mg (60% yield) of the title compound.

MS-ESI⁺ (m/z): 427

¹HNMR (500 MHz, CD₃OD; δ, ppm): 8.00 (m, 1H), 7.89 (m, 1H), 7.76 (m, 1H), 7.51 (m, 2H), 7.35 (m, 1H), 7.20 (m, 1H), 7.15 (m, 2H), 7.07 (m, 3H), 4.44 (m, 2H), 3.59 (m, 1H), 3.45 (m, 6H), 3.00 (m, 2H), 1.84 (m, 2H), 1.65 (m, 1H), 1.50 (m, 1H)

### Example 9

### Synthesis of (S)-4-methylnaphtalene-1-suphonic acid(5-amino-1-benzyl-oxymethylpentyl)amide (compound 9)

### Step I

Fmoc-L-Lys(Boc)-OH (300 mg, 468.54 g/mol 0.64 mmol, 1 eq), TEA (98 μl, 0.70 mmol, 1.1 eq), ethyl chloroformate (67 μl, 0.70 mmol, 1.1 eq) and sodium borohydride (36 mg, 0.96 mmol, 1.5 eq) were allowed to react according to the procedure described in Example 3, step I. In this manner, 175 mg (60% yield) of (S)-6-N-Boc-2-N′-Fmoc-2,6-diaminohexan-1-ol were obtained.

### Step II

(S)-6-N-Boc-2-N′-Fmoc-2,6-diaminohexan-1-ol (175 mg, 454.57 g/mol, 0.39 mmol, 1 eq) was treated in toluene with benzyl bromide (229 μl, 1.93 mmol, 5 eq) and silver(I) oxide (535 mg, 2.31 mmol, 6 eq) according to the procedure described in Example 3, step II, except that the reaction mixture was heated in an oil bath at 45° C. and that the reaction time was 20 h. 195 mg (93% yield) of (S)-6-benzyloxy-1-N-Boc-5-N′-Fmoc-hexane-1,5-diamine were obtained.

### Step III

(S)-6-Benzyloxy-1-N-Boc-5-N′-Fmoc-hexane-1,5-diamine (195 mg, 544.70 g/mol, 0.36 mmol) was treated with piperidine according to Example 2, step II. Purification by chromatography gave 48 mg (42% yield) of (S)-6-benzyloxy-1-N-Boc-hexane-1,5-diamine.

### Step IV

(S)-6-Benzyloxy-1-N-Boc-hexane-1,5-diamine (48 mg, 322.45 g/mol, 0.15 mmol, 1 eq) was sulfonylated with 4-methyl-1-naphtalene sulfonyl chloride (72 mg, 0.30 mmol, 2 eq) according to Example 3, step IV. In this manner, 47 mg (64% yield) of (S)-4-methylnaphtalene-1-suphonic acid [5-(N-Boc-amino)-1-benzyloxymethylpentyl]amide were obtained.

### Step V

(S)-4-Methylnaphtalene-1-suphonic acid [5-(N-Boc-amino)-1-benzyloxymethylpentyl]amide (32 mg, 526.70 g/mol, 66 μmol, 1 eq) was treated with TFA according to the procedure described in Example 3, step V, to yield 12 mg (42% yield) of the title compound.

MS-ESI⁺ (m/z): 427

¹HNMR (500 MHz, CD₃OD; δ, ppm): 8.75 (m, 1H), 8.16 (m, 1H), 8.13 (m, 1H), 7.64 (m, 2H), 7.40 (m, 1H), 7.23 (m, 3H), 7.08 (m, 2H), 4.16 (m, 2H), 3.29 (m, 1H), 3.27 (m, 1H), 3.15 (m, 1H), 2.75 (d, 3H), 2.34 (m, 2H), 1.47 (m, 1H), 1.36 (m, 1H), 1.17 (m, 2H), 1.1 (m, 1H), 0.97 (m, 1H)

### Example 10

### Synthesis of (S)-4-Methylnaphtalene-1-sulphonic acid (1-benzyloxy-methyl-5-isopropylaminopentyl)amide (compound 10)

(S)-4-Methylnaphtalene-1-suphonic acid (5-amino-1-benzyl-oxymethylpentyl)amide (21 mg, 426.58 g/mol, 45 μmol, 1 eq, Example 9) was dissolved in TMOF (1 ml). Acetone (11 μl, 58.08 g/mol, 0.79 g/cm³, 0.15 mmol, 3 eq), sodium triacetoxyborohydride (21 mg, 211.94 g/mol, 98 μmol, 2 eq), DIPEA (25 μl, 129.25 g/mol, 0.755 g/cm³, 0.15 mmol, 3 eq) and acetic acid (7 μl, 0.7 v-%) were added and the resulting mixture was stirred at room temperature for 5 h. The reaction mixture was evaporated and the product purified by chromatography to give 2.6 mg (11% yield) of the title compound in pure form.

MS-ESI⁺ (m/z): 469

¹HNMR (500 MHz, CD₃OD; δ, ppm): 8.72 (m, 1H), 8.18 (m, 1H), 8.12 (d, 1H), 7.67 (m, 2H), 7.41 (m, 1H), 7.21 (m, 3H), 6.97 (m, 2H), 3.98 (m, 2H), 3.28 (m, 2H), 3.06 (m, 1H), 2.95 (m, 1H), 2.76 (s, 3H), 2.75 (m, 2H), 1.54 (m, 2H), 1.46 (m, 2H), 1.33 (m, 1H), 1.29 (s, 6H), 1.25 (m, 1H)

### Example 11

### Synthesis of 4-methylnaphtalene-1-sulphonic acid (3-benzyloxy-1-piperidin-4-ylpropyl)amide (compound 11)

### Step I

3-(N-Fmoc-amino)-3-(N′-Boc-piperidin-4-yl)propionic acid (310 mg, 494.58 g/mol, 0.63 mmol, 1 eq, Pharmacore, UK), TEA (96 μl, 0.69 mmol, 1.1 eq), ethyl chloroformate (66 μl, 0.69 mmol, 1.1 eq) and sodium borohydride (36 mg, 0.94 mmol, 1.5 eq) were allowed to react according to the procedure described in Example 3, step I. In this manner, 189 mg (63% yield) of 3-(N-Fmoc-amino)-3-(N′-Boc-piperidin-4-yl)propan 1-ol were obtained.

### Step II

3-(N-Fmoc-amino)-3-(N′-Boc-piperidin-4-yl)propan-1-ol (72 mg, 480.61 g/mol, 0.15 mmol, 1 eq) was treated in toluene with benzyl bromide (99 μl, 0.83 mmol, 5.5 eq) and silver(I) oxide (241 mg, 1.04 mmol, 6.9 eq) according to the procedure described in Example 9, step II. After purification, 59 mg (69% yield) of 3-benzyloxy-1-(N-Boc-piperidin-4-yl)-N′-Fmoc-propylamine were obtained.

### Step III

3-Benzyloxy-1-(N-Boc-piperidin-4-yl)-N′-Fmoc-propylamine (59 mg, 570.74 g/mol, 0.10 mmol) was treated with piperidine according to Example 2, step II. Purification by chromatography gave 21 mg (59% yield) of 3-benzyloxy-1-(N-Boc-piperidin-4-yl)propylamine in pure form.

### Step IV

3-Benzyloxy-1-(N-Boc-piperidin-4-yl)propylamine (21 mg, 348.49 g/mol, 60 μmol, 1 eq) was reacted with 4-methyl-1-naphtalene sulfonyl chloride (22 mg, 90 μmol, 1.5 eq) according to the procedure described in Example 3, step IV, to obtain 24 mg (72% yield) of 4-methylnaphtalene-1-sulphonic acid [3-benzyloxy-1-(N-Boc-piperidin-4-yl)propyl]amide.

### Step V

4-Methylnaphtalene-1-sulphonic acid [3-benzyloxy-1-(N-Boc-piperidin-4-yl)propyl]amide (24 mg, 552.74 g/mol, 40 μmol) was treated with TFA according to the procedure described in Example 3, step V. In this manner 14 mg (73% yield) of the title compound were obtained.

MS-ESI⁺ (m/z): 453

¹HNMR (500 MHz, CD₃OD; δ, ppm): 8.76 (m, 1H), 8.18 (m, 1H), 8.10 (m, 1H), 7.67 (m, 2H), 7.37 (m, 1H), 7.26 (m, 3H), 7.07 (m, 2H), 3.92-3.70 (m, 3H), 3.19 (m, 1H), 3.08-2.87 (m, 3H), 2.75 (m, 1H), 2.71 (s, 3H), 2.45-2.34 (m, 1H), 1.98-1.81 (m, 1H), 1.61-1.49 (m, 3H), 1.38 (m, 1H), 1.29-1.15 (m, 2H)

### Example 12

### Synthesis of (S)-4-methylnaphthalene-1-sulfonic acid [4-amino-1-(4-methylbenzyloxymethyl)butyl]amide (compound 12)

### Step I

Boc-L-Ornithinol(Z) (291 mg, 0.82 mmol, 1 eq), was dissolved in dry toluene (2 ml) under argon atmosphere. Silver(I) oxide (277 mg, 1.20 mmol, 1.5 eq) was added, followed by 4-methylbenzyl bromide (298 mg, 185.06 g/mol, 1.61 mmol, 2.0 eq), which had been dissolved in toluene (1 ml, dry). After 4 d stirring at room temperature, the reaction mixture was evaporated and the residue purified by chromatography to give 217 mg (58% yield) of (S)-5-(4-methylbenzyloxy)-4-N-Boc-1-N′-Z-pentane-1,4-diamine.

### Step II

The Boc protection was removed by treating (S)-5-(4-methylbenzyloxy)-4-N-Boc-1-N′-Z-pentane-1,4-diamine (200 mg, 456.58 g/mol, 0.44 mmol) with TFA as described in Example 2, step IV. After chromatographic purification, 145 mg (68% yield) of (S)-5-(4-methylbenzyloxy)-1-N-Z-pentane-1,4-diamine were obtained as trifluoroacetic acid salt.

### Step III

(S)-5-(4-Methylbenzyloxy)-1-N′-Z-pentane-1,4-diamine trifluoroacetic acid salt (145 mg, 484.52 g/mol, 0.30 mmol, 1 eq) was sulfonylated with 4-methyl-1-naphthalenesulfonyl chloride (174.7 mg, 240.71 g/mol, 0.73 mmol, 2.4 eq) according to Example 3, step IV except that the reaction time was 2 d. After chromatographic purification, 160 mg (93% yield) of (S)-4-methylnaphthalene-1-sulfonic acid [4-(N-Z-amino)-1-(4-methylbenzyloxymethyl)butyl]amide were obtained.

### Step IV

For Z deprotection, (S)-4-methylnaphthalene-1-sulfonic acid [4-(N-Z-amino)-1-(4-methylbenzyloxymethyl)butyl]amide (160 mg, 576.76 g/mol, 0.28 mmol) was dissolved in methanol (10 ml), 10% Pd—C (80 mg) was added and the reaction was hydrogenated overnight under normal pressure and at room temperature. The reaction mixture was filtered and the filtrate evaporated. The residue was purified by preparative TLC to give 8.5 mg (7.1% yield) of the title compound in pure form.

MS-ESI⁺ (m/z): 427

### Example 13

### Synthesis of (S)-4-methylnaphthalene-1-sulfonic acid (4-amino-1-phenoxymethylbutyl)amide (compound 13)

### Step I

Boc-L-Ornithinol(Z) (1.50 g, 4.25 mmol, 1 eq), was dissolved in THF (8 ml, dry) and phenol (600 mg, 94.11 g/mol, 6.38 mmol, 1.5 eq) as well as triphenylphosphine (1.67 g, 6.38 mmol, 1.5 eq) were added. The mixture was bubbled with argon while DEAD (990 μl, 174.16 g/mol, 1.12 g/cm³, 6.38 mmol, 1.5 eq) dissolved in THF (4 ml, dry) was added dropwise to the mixture. After overnight stirring at room temperature, the reaction mixture was evaporated and the residue taken up in diethyl ether. The thus formed precipitate was removed by filtration and the filtrate was evaporated. The remaining residue was purified by chromatography to give 600 mg (33% yield) of (S)-4-N-Boc-5-phenoxy-1-N′-Z-pentane-1,4-diamine in pure form.

### Step II

The Boc protection was removed from (S)-4-N-Boc-5-phenoxy-1-N′-Z-pentane-1,4-diamine (600 mg, 428.53, 1.4 mmol) according to the procedure described in step IV of Example 2. After chromatographic purification, 418 mg (66% yield) of (S)-5-phenoxy-1-N-Z-pentane-1,4-diamine were obtained in form of its trifluoroacetic acid salt.

### Step III

(S)-5-Phenoxy-1-N-Z-pentane-1,4-diamine trifluoroacetic acid salt (418 mg, 455.46 g/mol, 0.92 mmol, 1 eq) was sulfonylated with 4-methyl-1-naphthalenesulfonyl chloride (527 mg, 240.71 g/mol, 2.19 mmol, 2.4 eq) according to the procedure described in step III of Example 12. After purification by chromatography, 210 mg (43% yield) of (S)-4-methylnaphthalene-1-sulfonic acid [4-(N-Z-amino)-1-phenoxymethylbutyl]amide were obtained in pure form.

### Step IV

The Z protection of (S)-4-methylnaphthalene-1-sulfonic acid [4-(N-Z-amino)-1-phenoxymethylbutyl]amide (207 mg, 532.66 g/mol, 0.39 mmol) was removed according to the procedure described in step IV of Example 12. Chromatographic purification yielded 54 mg (35% yield) of the title compound in pure form.

MS-ESI⁺ (m/z): 399

¹H NMR (500 MHz, CD₃OD; δ, ppm): 8.74 (m, 1H), 8.13 (m, 1H), 7.61 (m, 2H), 7.41 (m, 1H), 7.06 (m, 2H), 6.80 (m, 1H), 6.38 (m, 2H), 3.70-3.63 (m, 1H), 3.54 (m, 1H), 3.43 (m, 1H), 2.73 (s, 3H), 2.48 (m, 2H), 1.65-1.25 (m, 5H)

### Example 14

### Synthesis of (S)-4-methylnaphthalene-1-sulfonic acid [4-(1-ethylpropylamino)-1-phenoxymethylbutyl]amide (compound 14)

(S)-4-Methylnaphthalene-1-sulfonic acid (4-amino-1-phenoxymethylbutyl)amide (27 mg, 398.53 g/mol, 67 μmol, 1 eq, Example 13) was dissolved in TMOF (1.5 ml). 3-Pentanone (18 μl, 86.13 g/mol, 0.17 mmol, 2.6 eq), acetic acid (5.7 μl, 60.05 g/mol, 0.10 mmol, 1.5 eq), DIPEA (11 μl, 67 μmol, 1 eq) and finally sodium triacetoxyborohydride (35 mg, 0.165 mmol, 2.5 eq) were added to the reaction mixture. After 2 d of stirring at room temperature, the reaction mixture was evaporated and the residue purified by semi-preparative RP-HPLC to give 5.2 mg (17% yield) of the title compound in pure form.

MS-ESI⁺ (m/z): 469

### Example 15

### Synthesis of (S)-4-methyl-naphthalene-1-sulfonic acid (4-amino-1-benzyloxymethylbutyl)amide (compound 15)

### Step I

Boc-L-Ornithinol(Z) (303 mg, 0.86 mmol, 1 eq) was dissolved in dry toluene (2 ml) under argon atmosphere. Silver(I) oxide (405 mg, 1.75 mmol, 2.0 eq) was added to the reaction mixture, followed by benzyl bromide (308 μl, 2.60 mmol, 3.0 eq) prepared as a solution in toluene (2 ml, dry). After two days of stirring at room temperature, the reaction mixture was filtered and the filtrate evaporated. The obtained residue was purified by chromatography to obtain 227 mg (60% yield) of (S)-5-benzyloxy-4-N-Boc-1-N′-Z-pentane-1,4-diamine.

### Step II

The Boc protection was removed from (S)-5-benzyloxy-4-N-Boc-1-N′-Z-pentane-1,4-diamine (227 mg, 442.56 g/mol, 0.51 mmol) according to the procedure described in step III of Example 1 to obtain a quantitative amount of (S)-5-benzyloxy-1-N-Z-pentane-1,4-diamine in form of its trifluoroacetic acid salt.

### Step III

(S)-5-benzyloxy-1-N-Z-pentane-1,4-diamine trifluoroacetic acid salt (170 mg, 455.46 g/mol, 0.37 mmol, 1 eq) was sulfonylated with 4-methyl-1-naphthalenesulfonyl chloride (181 mg, 240.71 g/mol, 0.74 mmol, 2 eq) according to the procedure described in step III of Example 12. After chromatographic purification, 95 mg (46% yield) of (S)-4-methylnaphthalene-1-sulfonic acid [4-(N-Z-amino)-1-benzyloxymethylbutyl]amide were obtained.

### Step IV

The Z protection of (S)-4-methylnaphthalene-1-sulfonic acid [4-(N-Z-amino)-1-benzyloxymethylbutyl]amide (95 mg, 546.69 g/mol, 0.17 mmol) was removed according to the procedure described in step IV of Example 12. After chromatographic purification by preparative TLC, 15 mg (21% yield) of the title compound were obtained in pure form.

MS-ESI⁺ (m/z): 413

¹H NMR (500 MHz, CD₃OD; δ, ppm): 8.73 (m, 1H), 8.16 (m, 2H), 7.66 (m, 2H), 7.41 (m, 1H), 7.21 (m, 3H), 7.00 (m, 2H), 4.02 (m, 2H), 3.27 (m, 1H), 3.13 (m, 1H), 3.00 (m, 1H), 2.75 (m, 3H), 2.43 (m, 2H), 1.55-1.46 (m, 1H), 1.43-1.34 (m, 2H), 1.32-1.23 (m, 1H)

### Example 16

### Synthesis of (R)-[4-methylnaphthalene-1-sulfonic acid [1-(2-aminoethyl)-3-phenylpropyl]amide (compound 16)

### Step I

Rink amide resin (0.2 g, 0.7 mmol/g, 0.14 mmol) was washed twice with DMF prior to use. 3 ml of 20 vol-% piperidine in DMF was added to the resin and the mixture was agitated for 30 minutes. The piperidine/DMF-solution was removed by filtration and the treatment of the resin was repeated with fresh reagents. The resin was then washed thrice with DMF and thrice with DCM before it was used immediately for step II.

### Step II

Fmoc-(R)-3-amino-5-phenylpentanoic acid (175 mg, 415.49 g/mol, 0.42 mmol, 3 eq) and DIC (66 μl, 126.20 g/mol, 0.806 g/cm³, 0.42 mmol, 3 eq) were dissolved in dry DMF (1 ml) and allowed to stand for 10 minutes before they were mixed with the resin along with 1 ml of DCM. After overnight agitation the solvent was filtered off and the resin washed thrice with DMF and thrice with DCM.

### Step III

The Fmoc protection of the resin-attached amino acid obtained in step II was removed according to the procedure described in step I but without any washes prior to the treatment with piperidine/DMF.

### Step IV

4-Methyl-1-naphthalenesulfonyl chloride (100 mg, 240.71 g/mol, 0.42 mmol, 3 eq) was dissolved in dry THF (0.5 ml) and mixed with the amino acid-loaded resin obtained in step III. 20 vol-% of TEA in THF (1 ml) was then added to the mixture. After overnight agitation, the solvent was filtered off and the resin washed thrice with THF, thrice with DMF and finally thrice with DCM.

### Step V

The resin bound product of step IV was cleaved by treating the resin with 30 vol-% TFA in DCM (3 ml) for 30 min. The resulting red solution was collected and 1 ml water was added to it before the solvents were evaporated.

### Step VI

The product of step V was dissolved in BTHF (1 M, 2.0 ml) to reduce the carbonyl group. The reaction mixture was stirred overnight before being quenched by the addition of water (2.0 ml). The mixture was first made acidic with conc. HCl (2.0 ml) and stirred for 30 min before it was made alkaline with an NaOH solution (5 M) and the product was extracted with EtOAc. Drying and evaporation of the organic extract gave the title compound with 95% overall yield.

MS-ESI⁺ (m/z): 395

¹H NMR (500 MHz, CD₃OD; δ, ppm): 8.77 (m, 1H), 8.23 (m, 1H), 8.10 (m, 1H), 7.73 (m, 2H), 7.44 (m, 1H), 7.02 (m, 3H), 6.44 (m, 2H), 3.18 (m, 1H), 3.04 (m, 2H), 2.78 (s, 3H), 1.96-1.85 (m, 3H), 1.77-1.70 (m, 1H), 1.46-1.39 (m, 1H), 1.32-1.24 (m, 1H).

### Example 17

### Synthesis of (S)-4-methylnaphthalene-1-sulfonic acid {4-isopropylamino-1-[(1,2,3,4-tetrahydronaphthalen-1-ylamino)methyl]butyl}amide (compound 17)

The compound was synthesised according to the procedure described in Example 2, steps I-IV, with the exception that Fmoc-L-Dap(Boc)-OH was replaced with Fmoc-L-Orn(Boc)-OH, and benzyl amine with 1,2,3,4-tetrahydro-1-naphthylamine. The thus obtained (S)-4-methylnaphthalene-1-sulfonic acid [4-amino-1-(1,2,3,4-tetrahydronaphthalen-1-carbamoyl)butyl]amide was alkylated by reductive amination according to the procedure described in Example 10. The thus obtained (S)-4-methylnaphthalene-1-sulfonic acid [4-isopropylamino-1-(1,2,3,4-tetrahydronaphthalen-1-carbamoyl)butyl]amide was treated with BTHF according to the step V, Example 2. Finally, the crude product was purified by chromatography to give the title compound with 15% overall yield.

MS-ESI⁺ (m/z): 494

¹H NMR (500 MHz, CD₃OD; δ, ppm): ¹H NMR (500 MHz, CD₃OD; 8, ppm): 8.72 (m, 1H), 8.15 (m, 2H), 7.66 (m, 2H), 7.41 (m, 1H), 7.07 (m, 1H), 6.98 (m, 3H), 3.42 (m, 1H), 3.23 (m, 1H), 2.77 (s, 3H), 2.71 (m, 2H), 2.58 (m, 2H), 2.49-2.33 (m, 4H), 1.65-1.28 (m, 7H), 1.06 (d, 3H), 1.04 (d, 3H)

### Example 18

### Synthesis of (R)-4-methylnaphthalene-1-sulfonic acid [2-benzylsulfanyl-1-(2-dimethylaminoethylcarbamoyl)ethyl]amide (compound 18)

### Step I

N-Boc-S-benzyl-L-cysteine (502 mg, 311.40 g/mol, 1.61 mmol, 1 eq) was dissolved in dry DMF/DCM (1/1, 3 ml). HOBt (216 mg, 1.60 mmol, 1 eq) and DCC (203 mg, 206.33 g/mol, 0.99 mmol, 0.6 eq) were added to the reaction mixture. After 15 min, unsym-dimethylethylenediamine (0.180 ml, 88.15 g/mol, 0.8 g/cm³, 1.63 mmol, 1.01 eq) was added dropwise to the reaction mixture before it was stirred overnight at room temperature. The reaction mixture was then diluted with DCM and filtered. After evaporation of the solvent, the dry residue of the filtrate was taken up in DCM and washed twice with aq. sat. NaHCO₃ and water. The organic phase was dried over Na₂SO₄ and evaporated to yield 514 mg (84% yield) of (R)-2-(N-Boc-amino)-3-benzylsulfanyl-N′-(2-dimethylaminoethyl)propionamide.

### Step II

The Boc protection was removed by dissolving the (R)-2-(N-Boc-amino)-3-benzylsulfanyl-N′-(2-dimethylaminoethyl)propionamide (514 mg, 381.54 g/mol, 1.35 mmol) in 5 ml DCM containing 25 vol-% TFA. After 2 hours of stirring, the solvents were removed by evaporation and the residue was purified by chromatography to give 286 mg (53% yield) of (R)-2-amino-3-benzylsulfanyl-N-(2-dimethylaminoethyl)propionamide in form of its trifluoroacetic acid salt.

### Step III

(R)-2-amino-3-benzylsulfanyl-N-(2-dimethylaminoethyl)propionamide trifluoroacetic acid salt (286 mg, 395.45 g/mol, 0.72 mmol, 1 eq) was sulfonylated with 4-methyl-1-naphthalenesulfonyl chloride (367.5 mg, 1.53 mmol, 2.1 eq) according to the procedure described in Example 2, step III, to obtain 101 mg (50% yield) of the title compound in pure form.

MS-ESI⁺ (m/z): 486

¹H NMR (500 MHz, CD₃OD; δ, ppm): 8.73 (m, 1H), 8.19 (m, 1H), 8.16 (m, 1H), 7.72-7.66 (m, 2H), 7.46 (m, 1H), 7.20-7.15 (m, 3H), 7.02 (m, 2H), 3.78 (t, 1H, J=7.0 Hz), 3.34 (m, 2H), 3.09-3.00 (m, 2H), 2.77 (s, 3H), 2.57 (dd, 1H, J=14.0 Hz, J=7.0 Hz), 2.43 (dd, 1H, J=14.0 Hz, J=7.0), 2.23 (s, 6H), 2.22-2.12 (m, 2H).

### Example 19

### Synthesis of (R)-4-methylnaphthalene-1-sulfonic acid {2-benzylsulfanyl-1-[(2-dimethylaminoethylamino)methyl]ethyl}amide (compound 19)

### Step I

(R)-4-Methylnaphthalene-1-sulfonic acid [2-benzylsulfanyl-1-(2-dimethylaminoethylcarbamoyl)ethyl]amide (33 mg, 485.67 g/mol, 68 μmol, 1 eq, Example 18) was dissolved in THF (1 ml, dry) and BTHF (0.7 ml, 1.0 M) was added. The reaction mixture was refluxed for 4 d and then quenched by adding water (1.0 ml). The mixture was made acidic with HCl (1.0 ml, conc.) and refluxed for 30 min before it was made alkaline with aq. sat. NaHCO₃ solution and extracted with EtOAc. The organic extracts were dried over Na₂SO₄ and the solvent was evaporated. The thus prepared crude product was purified by chromatography to obtain the title compound in pure form with 27% yield (8.7 mg).

MS-ESI⁺ (m/z): 472

¹H NMR (500 MHz, CD₃OD; δ, ppm): 8.72 (m, 1H), 8.21 (m, 1H), 8.16 (d, 1H, J=7.5 Hz), 7.70 (m, 2H), 7.46 (d, 1H, J=7.5 Hz), 7.22-7.15 (m, 3H), 7.05 (m, 2H), 3.38-3.30 (m, 3H), 2.77 (d, 3H), 2.64 (m, 1H), 2.49 (m, 1H), 2.40-2.37 (m, 2H), 2.32 (m, 2H), 2.25 (s, 6H), 2.25-2.17 (m, 2H)

### Example 20

### Synthesis of (S)-4-methylnaphthalene-1-sulfonic acid [2-benzyloxy-1-(2-dimethylaminoethylcarbamoyl)ethyl]amide (compound 20)

The compound was synthesized according to the procedure described in Example 18, with the exception that instead of N-Boc-S-benzyl-L-cysteine, it was N-Boc-O-benzyl-L-serine that was used as the starting material. In this manner, the title compound was obtained in pure form with 8% overall yield.

MS-ESI⁺ (m/z): 470

### Example 21

### Synthesis of (R)-4-methylnaphthalene-1-sulfonic acid {2-benzyloxy-1-[(2-dimethylaminoethylamino)methyl]ethyl}amide (compound 21)

(S)-4-methylnaphthalene-1-sulfonic acid [2-benzyloxy-1-(2-dimethylaminoethylcarbamoyl)ethyl]amide (23 mg, 469.61 g/mol, 49 μmol, 1 eq, Example 20) was treated with BTHF (0.75 ml, 0.74 mmol, 15 eq) according to the procedure described in Example 19 to obtain 11 mg (50% yield) of the title compound in pure form.

MS-ESI⁺ (m/z): 456

¹H NMR (500 MHz, CD₃OD; δ, ppm): 8.72 (m, 1H), 8.17 (m, 2H), 7.68 (m, 2H), 7.42 (m, 1H), 7.22 (m, 3H), 7.02 (m, 2H), 4.05 (s, 2H), 3.42 (m, 1H), 3.18 (dd, 1H, J=4.5 Hz, J=9.7 Hz), 3.06 (dd, 1H, J=6.1 Hz, J=9.7 Hz), 2.76 (s, 3H), 2.66-2.58 (m, 2H), 2.54-2.45 (m, 2H), 2.29-2.24 (m, 2H), 2.25 (s, 6H)

### Example 22

### Synthesis of (S)—N-(3-amino-1-benzyloxymethylpropyl)-2,3,4,5,6-pentamethylbenzenesulfonamide (compound 22)

The compound was synthesised according to the procedure described in Example 3, with the exception that 4-methyl-1-naphthalenesulfonyl chloride was substituted by pentamethylbenzenesulphonyl chloride. In this manner, the title compound was obtained with 25% yield.

MS-ESI⁺ (m/z): 405

¹H NMR (500 MHz, CD₃OD; δ, ppm): 7.25 (m, 3H), 7.14 (m, 2H), 4.25 (s, 2H), 3.44 (m, 1H), 3.23 (dd, 1H, J=4.5 Hz, J=9.5 Hz), 3.14 (dd, 1H, J=5.7 Hz, J=9.5 Hz), 2.77-2.67 (m, 2H), 2.55 (s, 6H), 2.27 (s, 3H), 2.21 (s, 6H), 1.74-1.62 (m, 2H).

### Example 23

### Synthesis of (S)—N-(1-benzyloxymethyl-3-guanidinylpropyl)-2,3,4,5,6-pentamethylbenzenesulfonamide (compound 23)

(S)—N-(3-amino-1-benzyloxymethylpropyl)-2,3,4,5,6-pentamethylbenzenesulfonamide (39 mg, 404.57 g/mol, 96 μmol, Example 22) was guanidylated according to the procedure described in Example 4, step I. The thus formed (S)—N-(1-benzyloxymethyl-3-(N,N′-bis(Boc)guanidinylpropyl)-2,3,4,5,6-pentamethylbenzenesulfonamide (58 mg, 646.85 g/mol, 89 μmol) was dissolved in DCM (5 ml), TFA (1.7 ml) was added and the resulting mixture was stirred for 4.5 h at room temperature. The reaction mixture was then diluted with DCM (15 ml) and washed twice with water and brine. The organic phase was dried over Na₂SO₄ and the solvent evaporated to obtain 41 mg (82% yield) of the title compound in form of its trifluoroacetic acid salt.

MS-ESI⁺ (m/z): 447

¹H NMR (500 MHz, CD₃OD; δ, ppm): 7.25 (m, 3H), 7.13 (m, 2H), 4.25 (s, 2H), 3.44 (m, 1H), 3.29 (m, 2H), 3.22 (m, 1H), 3.13 (m, 1H), 2.55 (s, 6H), 2.28 (s, 3H), 2.21 (s, 6H), 1.86 (m, 1H), 1.75 (m, 1H).

### Example 24

### Synthesis of (R)-4-methylnaphthalene-1-sulfonic acid [1-(2-aminoethylcarbamoyl)-2-benzyloxyethyl]amide (compound 24)

The compound was synthesised according to the procedure described in Example 18, with the exception that in step I N-Boc-S-benzyl-L-cysteine and unsym-dimethylethylenediamine were replaced with N-Boc-O-benzyl-D-serine and ethylenediamine, respectively. Furthermore, the thus in step I obtained (R)—N-(2-aminoethyl)-2-(N′-Boc-amino)-3-benzyloxypropionamide was Fmoc protected before the steps II-III described in Example 18 were carried out. The Fmoc protection was removed after step III and the product was then purified by chromatography to obtain 21 mg (5% overall yield) of the title compound in pure form.

MS-ESI (m/z): 442

¹H NMR (500 MHz, CD₃OD; δ, ppm): 8.72 (m, 1H), 8.18 (m, 1H), 8.14 (d, 2H, J=7.5 Hz), 7.68 (m, 2H), 7.42 (d, 1H, J=7.5 Hz), 7.21 (m, 3H), 7.00 (m, 2H), 4.12 (s, 2H), 3.85 (t, 1H, J=5.3 Hz), 3.49 (dd, 1H, J=5.3 Hz, J=9.7 Hz), 3.25 (dd, 1H, J=5.3 Hz, J=9.7 Hz), 3.07 (t, 2H, J=6.3 Hz), 2.76 (s, 3H), 2.54 (t, 2H, J=6.3 Hz)

### Example 25

### Synthesis of (S)-2,3,4,5,6-pentamethylbenzenesulfonic acid [2-benzyloxy-1-(2-dimethylaminoethylcarbamoyl)ethyl]amide (compound 25)

The compound was synthesised according to the procedure described in Example 18, with the exception that in step I N-Boc-S-benzyl-L-cysteine was substituted with N-Boc-O-benzyl-L-serine (500 mg, 295.34 g/mol, 1.69 mmol, 1 eq) and in step III pentamethylsulphonyl chloride (97 mg, 246.76 g/mol, 0.39 mmol, 1.3 eq) was used instead of 4-methyl-1-naphthalenesulfonyl chloride. Chromatographic purification gave the title compound with 18% overall yield (145 mg).

MS-ESI⁺ (m/z): 512

¹H NMR (500 MHz, CD₃OD; δ, ppm): 7.24 (m, 3H), 7.16 (m, 2H), 4.30 (m, 2H), 3.79 (t, 1H, J=5.3 Hz), 3.59 (dd, 1H, J=5.3 Hz, J=9.6 Hz), 3.38 (dd, 1H, J=5.3 Hz, J=9.6 Hz), 3.20 (td, 2H, J=6.8 Hz, J=2.0 Hz), 2.51 (s, 6H), 2.31 (t, 2H, J=6.8 Hz), 2.25 (s, 3H), 2.21 (s, 6H), 2.17 (s, 6H)

### Example 26

### Synthesis of (S)-4-methylnaphthalene-1-sulfonic acid [1-(2-amino-2-methylpropylcarbamoyl)-2-benzyloxyethyl]amide (compound 26)

### Step I

The Boc protection of N-Boc-O-benzyl-L-serine (1.09 g, 3.7 mmol, 1 eq) was removed with TFA according to the procedure described in Example 6, step II. The thus obtained crude trifluoro acetic acid salt of (S)-2-amino-3-benzyloxy-propionic acid was used in the next step without further purification.

### Step II

The trifluoroacetic acid salt of (S)-2-Amino-3-benzyloxy-propionic acid (3.7 mmol. 1 eq) was sulfonylated with 4-methyl-1-naphthalenesulfonyl chloride (1.55 g, 6.4 mmol, 1.7 eq) according to the procedure described in Example 3, step IV. In this manner, 641 mg (44% yield) of (S)-3-benzyloxy-2-(4-methylnaphthalene-1-sulfonylamino)propionic acid were obtained.

### Step III

(S)-3-benzyloxy-2-(4-methylnaphthalene-1-sulfonylamino)propionic acid (160 mg, 399.74 g/mol, 0.40 mmol) was dissolved in DCM (3 ml). DCC (82 mg, 0.40 mmol, 1 eq) and 1,2-diamino-2-methylpropane (62 μl, 88.15 g/mol, 0.847 g/cm³, 0.60 mmol, 1.5 eq) were added, and the resulting mixture was stirred at room temperature for 3 d. The reaction mixture was then filtered, the solvent evaporated and the obtained residue purified by chromatography to obtain 8.4 mg (4% yield) of the title compound.

MS-ESI⁺ (m/z): 470

### Example 27

### Synthesis of (S)-4-methylnaphthalene-1-sulfonic acid {2-benzyloxy-1-[(2-dimethylaminoethylamino)methyl]ethyl}amide (compound 27)

The compound was synthesised according to the procedure described in Example 18, except that in step I N-Boc-S-benzyl-L-cysteine was substituted by N-Boc-O-benzyl-D-serine (298 mg, 1.0 mmol). The thus obtained (S)-4-methylnaphthalene-1-sulfonic acid [2-benzyloxy-1-(2-dimethylaminoethylcarbamoyl)ethyl]amide (21 mg, 469.61 g/mol, 45 μmol) was treated with BTHF according to the procedure described in Example 19. Finally, 3.8 mg (1% overall yield) of the title compound were obtained in pure form.

MS-ESI⁺ (m/z): 456

1H NMR (500 MHz, CD₃OD; δ, ppm): 8.69 (m, 1H), 8.16 (m, 1H), 8.12 (m, 1H), 7.65 (m, 2H), 7.39 (m, 1H), 7.20 (m, 3H), 7.00 (m, 2H), 4.04 (s, 2H), 3.42-3.36 (m, 1H), 3.17 (m, 1H), 3.05 (m, 1H), 2.73 (s, 3H), 2.56 (m, 2H), 2.40 (m, 2H), 2.14 (s, 6H), 2.18-2.12 (m, 2H)

### Example 28

### Synthesis of (R)-4-methylnaphthalene-1-sulfonic acid [2-benzyloxy-1-(2-morpholin-4-ylethylcarbamoyl)ethyl]amide (compound 28)

The compound was synthesised according to the procedure described in Example 18, except that N-Boc-S-benzyl-L-cysteine and unsym-dimethylethylenediamine were replaced with N-Boc-O-benzyl-D-serine (150 mg, 0.51 mmol. 1 eq) and 4-(2-aminoethyl)morpholine (108 μl, 130.19 g/mol, 0.922 g/cm³, 0.77 mmol, 1.5 eq), respectively. In this manner, 115 mg (44% overall yield) of the title compound were obtained in pure form.

MS-ESI⁺ (m/z): 512

1H NMR (500 MHz, CD₃OD; δ, ppm): 8.74 (m, 1H), 8.17 (m, 1H), 8.13 (m, 1H), 7.68 (m, 2H), 7.42 (m, 1H), 7.20 (m, 3H), 7.01 (m, 2H), 4.12 (s, 2H), 3.87 (m, 1H), 3.63 (m, 4H), 3.51 (m, 1H), 3.26 (m, 1H), 3.12 (m, 2H), 2.76 (s, 3H), 2.38 (m, 4H), 2.23 (m, 2H).

### Example 29

### Synthesis of (S)-4-methylnaphthalene-1-sulfonic acid [2-benzyloxy-1-(2-morpholin-4-ylethylamino)ethyl]amide (compound 29)

(R)-4-Methylnaphthalene-1-sulfonic acid [2-benzyloxy-1-(2-morpholin-4-ylethylcarbamoyl)ethyl]amide (109 mg, 511.64 g/mol, 0.21 mmol, Example 28) was treated with BTHF according to the procedure described in Example 19 to give 66 mg (63% yield) of the title compound in pure form,

MS-ESI⁺ (m/z): 498

¹H NMR (500 MHz, CD₃OD; δ, ppm): 8.72 (m, 1H), 8.18 (m, 1H), 8.15 (m, 1H), 7.68 (m, 2H), 7.42 (m, 1H), 7.19-7.24 (m, 3H), 7.99 (m, 2H), 4.00 (s, 2H), 3.65 (m, 4H), 3.45 (m, 1H), 3.12 (dd, 1H, J=4.2 Hz, J=9.8 Hz), 3.01 (dd, 1H, J=6.2 Hz, J=9.8 Hz), 2.76 (m, 3H), 2.66 (m, 1H), 2.59 (m, 1H), 2.54 (m, 1H), 2.45 (m, 1H), 2.35-2.42 (m, 4H), 2.24 (m, 2H)

### Example 30

### Synthesis of (S)-4-methylnaphthalene-1-sulfonic acid [4-amino-1-(4-trifluoromethoxybenzyloxymethyl)butyl]amide (compound 30)

The compound was synthesized according to the procedure described in Example 12, except that instead of 4-methylbenzyl bromide, 4-trifluoromethoxybenzyl bromide was used. In this manner, the title compound was obtained in pure form with 8% overall yield.

MS-ESI⁺ (m/z): 497

1H NMR (500 MHz, CD₃OD; δ, ppm): 8.71 (m, 1H), 8.16 (m, 1H), 8.12 (m, 1H), 7.66 (m, 2H), 7.41 (m, 1H), 7.08 (m, 2H), 6.97 (m, 2H), 3.89 (m, 2H), 3.36 (m, 1H), 3.01 (m, 1H), 2.89 (m, 1H), 2.88-2.79 (m, 2H), 2.74 (s, 3H), 1.80-1.50 (m, 4H)

### Example 31

### Synthesis of (S)-4-methylnaphthalene-1-sulfonic acid [1-(2-aminoethylcarbamoyl)-2-benzylsulfanylethyl]amide (compound 31)

The compound was synthesized according to the procedure described in Example 18, except that instead of N-Boc-S-benzyl-L-cysteine and unsym-dimethylethylenediamine, N-Boc-S-benzyl-D-cysteine (303 mg, 311.40 g/mol, 0.97 mmol, 1 eq) and ethylenediamine (295 μl, 60.10 g/mol, 0.897 g/cm³, 4.4 mmol, 4.5 eq) were used. The title compound was obtained in pure form with 8% overall yield (20 mg).

MS-ESI⁺ (m/z): 458

1H NMR (500 MHz, CD₃OD; δ, ppm): 8.73 (m, 1H), 8.21 (m, 1H), 8.17 (m, 1H), 7.70 (m, 2H), 7.48 (m, 1H), 7.14 (m, 3H), 6.94 (m, 2H), 3.74 (m, 1H), 3.27 (m, 2H), 3.12 (m, 2H), 2.79 (s, 3H), 2.67 (m, 2H), 2.56 (m, 1H), 2.38 (m, 1H).

### Example 32

### Synthesis of 4-methylnaphthalene-1-sulfonic acid (1-benzyloxymethyl-2-pyrrolidin-2-ylethyl)amide (compound 32)

### Step I

2-Amino-3-(N-Boc-pyrrolidin-2-yl)propan-1-ol (151 mg, 244.33 g/mol, 0.62 mmol, 1 eq, Pharmacore, UK) was dissolved in THF (3 ml, dry). TEA (81 μl, 0.59 mmol, 0.95 eq) and 4-methyl-1-naphtalene sulfonyl chloride (140 mg, 0.59 mmol, 0.95 eq) were added and the resulting mixture was stirred overnight at room temperature. The reaction mixture was evaporated and purified by column chromatography to give 159 mg (58% yield) of 4-methylnaphthalene-1-sulfonic acid (1-hydroxymethyl-2-pyrrolidin-2-ylethyl)amide.

### Step II

4-Methylnaphthalene-1-sulfonic acid (1-hydroxymethyl-2-pyrrolidin-2-ylethyl)amide (159 mg, 448.59 g/mol, 0.35 mmol, 1 eq) was treated with benzyl bromide (210 μl, 1.76 mmol, 5 eq) and silver(I) oxide (405 mg, 1.76 mmol, 5 eq) according to the procedure described in Example 3, step II. In this manner, 141 mg (75% yield) of 4-methylnaphthalene-1-sulfonic acid [1-benzyloxymethyl-2-(N-Boc-pyrrolidin-2-yl)ethyl]amide were obtained.

### Step III

The Boc protection was removed by treating 4-methylnaphthalene-1-sulfonic acid [1-benzyloxymethyl-2-(N-Boc-pyrrolidin-2-yl)ethyl]amide (141 mg, 538.71 g/mol, 0.26 mmol) with TFA according to the procedure described in Example 1, step III. In this manner, 73 mg (22% overall yield) of the title compound were obtained as trifluoroacetic acid salt.

MS-ESI⁺ (m/z): 439

¹H NMR (500 MHz, CD₃OD; δ, ppm): 8.71 (m, 1H), 8.13-8.23 (m, 2H), 7.71 (m, 2H), 7.40-7.47 (m, 1H), 7.15-7.28 (m, 3H), 6.85-7.0 (m, 2H), 3.98 (m, 2H), 3.42 (m, 1H), 3.26-3.30 (m, 1H), 3.18 (m, 1H), 3.10 (m, 1H), 3.05 (m, 1H), 2.91 (m, 1H), 2.78 (s, 3H), 1.88-1.97 (m, 2H), 1.76 (m, 2H), 1.66 (m, 1H), 1.38 (m, 1H)

### Example 33

### Synthesis of (S)-4-methylnaphthalene-1-sulfonic acid (4-isopropylamino-1-phenoxymethylbutyl)amide (compound 33)

(S)-4-Methylnaphthalene-1-sulfonic acid (4-amino-1-phenoxymethylbutyl)amide (26 mg, 398.53 g/mol, 66 μmol, 1 eq, Example 13) was dissolved in TMOF (1.5 ml). Acetone (14 μl, 0.19 mmol, 2.9 eq), sodium triacetoxyborohydride (29 mg, 0.14 mmol, 2.1 eq) and acetic acid (6 μl, 99 μmol, 1.5 eq) were added and the reaction mixture was stirred overnight at room temperature. Then DIPEA (11 μl, 67 μmol, 1 eq) was added and the stirring of the reaction mixture was continued for another night. The solvents were evaporated, the residue was taken up in EtOAc and washed with water. The aqueous phase was made alkaline by adding sat. aq. NaHCO₃-solution before it was extracted with EtOAc. The combined organic phase was dried over Na₂SO₄ and the solvent was evaporated. The residue was purified by semi-preparative RP-HPLC to yield 8.1 mg (28% yield) of the title compound in pure form.

MS-ESI⁺ (m/z): 441

### Example 34

### Synthesis of (S)-4-methylnaphthalene-1-sulfonic acid (4-amino-1-phenylsulfanylmethylbutyl)amide (compound 67)

### Step I

Boc-L-Om(Z)-OH (2.0 g, 366.4 g/mol, 5.46 mmol, 1.0 eq) was reduced according to the procedure described in Example 3, step I. The thus obtained (S)-2-N-Boc-5-N′-Z-2,5-diaminopentan-1-ol 1.5 g (76% yield) was used without further purification in the next reaction step.

### Step II

(S)-2-N-Boc-5-N′-Z-2,5-diaminopentan-1-ol (1.1 g, 352.43 g/mol, 3.19 mmol, 1.0 eq) was chlorinated according to the procedure described in Example 1, step I. The reaction product was purified by silica gel chromatography to obtain 0.5 g (42% yield) of (S)-5-chloro-4-N-Boc-1-N′-Z-pentane-1,4-diamine.

### Step III

(S)-5-chloro-4-N-Boc-1-N′-Z-pentane-1,4-diamine (96 mg, 370.88 g/mol, 0.26 mmol, 1.0 eq.) was reacted with thiophenol (53 ml, 1.08 g/cm3, 110.17 g/mol, 0.52 mmol, 2.0 eq.) according to the procedure described in Example 1, step II except that the reaction time was 2 d. The reaction product was purified by silica gel chromatography to obtain 79 mg (69% yield) of (S)-5-phenylsulfanyl-4-N-Boc-1-N′-Z-pentane-1,4-diamine.

### Step IV

The Boc protection was removed by dissolving (S)-5-phenylsulfanyl-4-N-Boc-1-N′-Z-pentane-1,4-diamine (79 mg, 444.6 g/mol, 0.18 mmol) in 4 ml DCM containing 20 vol % TFA. The reaction mixture was stirred 30 minutes at room temperature and evaporated to dryness. The obtained crude (S)-5-phenylsulfanyl-1-N-Z-pentane-1,4-diamine trifluoroacetic acid salt was used without further purification in the next reaction step.

### Step V

(S)-5-phenylsulfanyl-1-N-Z-pentane-1,4-diamine trifluoroacetic acid salt (104 mg, 344.48 g/mol, 0.30 mmol, 1.0 eq.) was sulfonylated with 4-methyl-1-naphthalene sulfonyl chloride (124 mg, 240.71 g/mol, 0.51 mmol, 1.7 eq.) according to the procedure in Example 3, step IV. The reaction product was purified by silica gel chromatography to obtain 83.9 mg (51% yield) of (S)-4-methylnaphthalene-1-sulfonic acid [4-(N-Z-amino)-1-phenylsulfanylmethylbutyl]amide,

### Step VI

The Z protection was removed from (S)-4-methylnaphthalene-1-sulfonic acid [4-(N-Z-amino)-1-phenylsulfanylmethylbutyl]amide (83.9 mg, 548.73 g/mol, 0.15 mmol) according to the procedure described in Example 1, step V. The reaction product was purified by silica gel chromatography to obtain 33 mg (53% yield) of the title compound.

MS-ESI⁺ (m/z): 415

1H NMR (500 MHz, CD₃OD; d, ppm): 8.70 (m, 1H), 8.19 (m, 1H), 7.96 (d, 1H, J=7.57 Hz), 7.70 (m, 2H), 7.36 (d, 1H, J=7.57 Hz), 7.15 (m, 3H), 6.80 (m, 2H), 3.18-3.13 (m, 1H), 2.77 (s, 3H), 2.78-2.69 (m, 2H), 2.55 (m, 1H), 1.86 (m, 1H), 1.55 (m, 1H), 1.59-1.50 (m, 1H), 1.43 (m, 1H)

### Example 35

### Synthesis of (S)-4-methylnaphthalene-1-sulfonic acid [4-amino-1-(isoquinolin-6-yloxymethylbutyl)]-amide (compound 69)

### Step I

Boc-L-Ornithinol(Z) (1.13 g, 352.43 g/mol, 3.19 mmol, 1.0 eq.) was dissolved in THF (8 ml) together with 7-hydroxyisoquinoline (601 mg, 145.16 g/mol, 4.15 mmol) and triphenylphosphine (1.1 g, 262.29 g/mol, 4.15 mmol, 1.3 eq) before DEAD (644 μl, 174.16 g/mol, 1.12 g/cm³, 0.59 mmol, 1.5 eq) was added in a dropwise fashion. After reacting for 4.5 hours at room temperature, the reaction mixture was evaporated to dryness and the residue was purified by silica gel chromatography to provide 722 mg (47% yield) of (S)-5-isoquinolin-6-yloxy-4-N-Boc-1-N′-Z-pentane-1,4-diamine.

### Step II

The Boc protection was removed by treating (S)-5-isoquinolin-6-yloxy-4-N-Boc-1-N′-Z-pentane-1,4-diamine (722 mg, 479.58 g/mol, 1.5 mmol) with TFA according to the procedure described in Example 3, step V. After silica gel chromatography purification 440 mg (77% yield) of (S)-5-isoquinolin-6-yloxy-1-N-Z-pentane-1,4-diamine was obtained.

### Step III

(S)-5-isoquinolin-6-yloxy-1-N′-Z-pentane-1,4-diamine (280 mg, 379.46 g/mol, 0.74 mmol 1.0 eq.) was sulfonylated with 4-methyl-1-naphthalenesulfonyl chloride (273 mg, 240.71 g/mol, 1.11 mmol, 1.5 eq.) according to the procedure described in Example 3, step IV. After silica gel chromatography purification 99 mg (23% yield) of (S)-4-methylnaphthalene-1-sulfonic acid [4-(N-Z-amino)-1-(isoquinolin-6-yloxymethyl)-butyl]-amide was obtained.

### Step IV

Z-protection was removed by dissolving (S)-4-methylnaphthalene-1-sulfonic acid [4-(N-Z-amino)-1-(isoquinolin-6-yloxymethyl)-butyl]-amide (99 mg, 583.71 g/mol, 0.17 mmol) in MeCN (2.0 ml), followed by the addition of iodotrimethylsilane (61 μl, 200.09 g/mol, 1.4 g/cm³, 1.1 mmol, 2.5 eq). After a reaction time of 1.5 hour the mixture was evaporated to dryness. The residue was dissolved in DCM and was washed with 10% Na₂SO₃. The organic phase was then dried over Na₂SO₄ and evaporated. After work-up, 69 mg (90% yield) of the title compound were obtained.

MS-ESI⁺ (m/z): 450

¹H NMR (500 MHz, CD₃OD; δ, ppm): 9.02 (s, 1H), 8.69 (d, 1H, J=8.29 Hz), 8.32 (d, 1H, J=5.64), 8.07 (d, 1H, J=7.63), 8.02 (d, 1H, J=8.29), 7.65 (d, 1H, J=5.64), 7.54 (m, 3H), 7.39 (d, 1H, J=7.63 Hz) 6.95 (m, 1H), 6.42 (m, 1H), 3.80 (m, 2H), 3.53 (m, 1H), 2.72 (m, 2H), 2.60 (s, 3H), 1.74-1.57 (m, 4H)

### Example 36

### Synthesis of (R)-4-methylnaphthalene-1-sulfonic acid [2-benzyloxy-2-(S)methyl-1-(aminoethylcarbamoyl)-ethyl]amide (compound 70)

### Step I

Boc-O-benzyl-D-threonine (500 mg, 309.4 g/mol, 1.62 mmol, 1.0 eq) was dissolved in dry DCM. DCC (336.6 mg, 206.33 g/mol, 1.62 mmol, 1.0 eq) was added and the mixture was stirred briefly, before ethylenediamine (270 μl, 60.10 g/mol, 0.90 g/cm³, 4.04 mmol, 2.5 eq) dissolved in DCM was added slowly to the solution. The reaction mixture was stirred 45 minutes at room temperature. The formed precipitate was filtered off and the filtrate was evaporated to dryness. The reaction product was purified by silica gel chromatography to obtain 195 mg (34% yield) of (R)-2-(N-Boc-amino)-3-benzyloxy-3-(S)-methyl-N′-(2-ethylamino)propionamide.

### Step II

(R)-2-(N-Boc-amino)-3-benzyloxy-3-(S)-methyl-N′-(2-ethylamino)propionamide (194 mg, 351.45 g/mol, 0.55 mmol, 1.0 eq) was dissolved in DCM. DIPEA (142 μl, 129.25 g/mol, 0.83 mmol, 1.5 eq) was added and the mixture was stirred 10 minutes before 9-fluorenylmethyl-chloroformate (158 mg, 258.70 g/mol, 0.61 mmol, 1.1 eq) was added. The reaction mixture was stirred 2 hours at room temperature and then evaporated to dryness. The reaction product was purified by silica gel chromatography to obtain 268 mg (85% yield) of (R)-2-(N-Boc-amino)-3-benzyloxy-3-(S)-methyl-N′-(2-N″-Fmoc-aminoethyl)propionamide.

### Step III

The Boc protection was removed by treating (R)-2-(N-Boc-amino)-3-benzyloxy-3-(S)-methyl-N′-(2-N′-Fmoc-aminoethyl)propionamide (285 mg, 573.70 g/mol, 0.50 mmol) with TFA according to the procedure described in Example 3, step V. The reaction product was purified by silica gel chromatography to obtain 263 mg (89% yield) of (R)-2-amino-3-benzyloxy-3-(S)-methyl-N-(2-N′-Fmoc-aminoethyl)propionamide.

### Step IV

(R)-2-amino-3-benzyloxy-3-(S)-methyl-N-(2-N″-Fmoc-aminoethyl)propionamide (135 mg, 586.59 g/mol, 0.23 mmol, 1.0 eq) was sulfonylated with 4-methyl-1-naphthalenesulfonyl chloride (83.6 mg, 240.71 g/mol, 0.35 mmol, 1.5 eq.) according to the procedure described in Example 3, step IV. The reaction product was purified by silica gel chromatography to obtain 122 mg (78% yield) of (R)-4-methyl-naphthalene-1-sulfonic acid [2-benzyloxy-2-(S)-methyl-1-(2-N-Fmoc-aminoethylcarbamoyl)-ethyl]amide.

### Step V

The Fmoc was removed from (R)-4-methyl-naphthalene-1-sulfonic acid [2-benzyloxy-2-(S)-methyl-1-(2-N-Fmoc-aminoethylcarbamoyl)ethyl]amide as described in Example 2, step II. The reaction product was purified by silica gel chromatography to obtain 57 mg (72%) of the title compound in pure form.

MS-ESI⁺ (m/z): 456

¹H NMR (500 MHz, (CD₃OD; δ, ppm): 8.78 (m, 1H), 8.18 (m, 1H), 8.12 (d, 1H, J=7.46 Hz), 7.68 (m, 2H), 7.42 (m, 1H), 7.25 (m, 3H), 7.16 (m, 2H), 4.42 (d, 1H, J=11.82 Hz), 4.29 (d, 1H, J=11.82 Hz), 3.79 (m, 1H), 3.64 (d, 1H, J=3.98 Hz), 3.04 (m, 2H), 2.76 (s, 3H), 2.50 (m, 2H), 0.82 (d, 3H, J=6.30 Hz)

### Example 37

### Synthesis of (R)-4-methylnaphthalene-1-sulfonic acid [2-benzyloxy-2-(S)methyl-1-(2-isopropylaminoethylcarbamoyl)-ethyl]amide (compound 71) Step I-V

(R)-4-Methylnaphthalene-1-sulfonic acid [2-benzyloxy-2-(S)methyl-1-(aminoethylcarbamoyl)-ethyl]amide was prepared according to the procedure described in Example 36 steps I-V.

### Step VI

(R)-4-Methylnaphthalene-1-sulfonic acid [2-benzyloxy-2-(S)-methyl-1-(aminoethylcarbamoyl)-ethyl]amide (47 mg, 455.58 g/mol, 0.10 mmol, 1.0 eq) was dissolved in TMOF. Acetone (15 μl, 58.08 g/mol, 0.21 mmol, 2.0 eq), sodium triacetoxyborohydride (48.2 mg, 211.94 g/mol, 0.23 mmol, 2.2 eq) and acetic acid (8.8 μl, 60.05 g/mol, 0.16 mmol, 1.5 eq) were added and the reaction mixture was stirred overnight at room temperature. The reaction mixture was then evaporated to dryness and the residue was dissolved in DCM. The organic phase was washed twice with sat. aq. NaHCO₃-solution, once with brine, dried over Na₂SO₄, filtered and evaporated. The reaction product was purified by silica gel chromatography to obtain 37 mg (73% yield) of the title compound in pure form.

MS-ESI⁺ (m/z): 498

¹H NMR (500 MHz, (CD₃OD; δ, ppm): 8.78 (m, 1H), 8.17 (m, 1H), 8.11 (d, 1H, J=7.57 Hz), 7.68 (m, 2H), 7.42 (m, 1H), 7.28-7.22 (m, 3H), 7.16 (m, 2H), 4.42 (d, 1H, J=11.70 Hz), 4.30 (d, 1H, J=11.70 Hz), 3.79 (m, 1H), 3.63 (d, 1H, J=3.94), 3.10 (m, 2H), 2.76 (s, 3H), 2.72 (m, 1H), 2.43 (t, 2H, J=6.54 Hz), 1.01 (dd, 6H, J=2.36 Hz, J=6.30 Hz), 0.82 (d, 3H, J=6.30 Hz)

### Example 38

### Synthesis of (R)-4-methylnaphtalene-1-sulfonic acid [1-(2-dimethylamino-ethylcarbamaoyl)-4-phenylbut-3-enyl]amide (compound 72)

### Step I

Boc-styrylalanine.DCHA (473 mg, 472.67 g/mol, 1.0 mmol, 1.0 eq) was coupled with unsym-dimethylethylenediamine (132 μl, 88.15 g/mol, 0.8 g/cm³, 1.2 mmol, 1.2 eq.) according to the procedure described in Example 2, step I. After 2 days, the reaction mixture was evaporated to dryness and the residue was dissolved in DCM. The organic phase was washed with sat. aq. NaHCO₃-solution and water before it was dried over Na₂SO₄, filtered and evaporated. The thus obtained (R)-2-N-boc-amino-5-phenylpent-4-enoic acid (2-dimethylamino-ethyl)amide was used without further purification in the next reaction step.

### Step II

The Boc protection was removed from (R)-2-N-boc-amino-5-phenylpent-4-enoic acid (2-dimethylamino-ethyl)amide (517 mg, 361.48 g/mol, 1.43 mmol) according to the procedure described in Example 3, step V. The thus obtained (R)-2-amino-5-phenylpent-4-enoic acid (2-dimethylaminoethyl)amide was used without further purification in the next reaction.

### Step III

(R)-2-Amino-5-phenylpent-4-enoic acid (2-dimethylaminoethyl)amide (261 mg, 261.37 g/mol, 1.0 mmol, 1.0 eq.) was sulfonylated with 4-methyl-1-naphthalenesulfonyl chloride (444 mg, 240.71 g/mol, 1.7 mmol, 1.7 eq.) according to the procedure described in Example 3, step IV. The reaction product was purified once by silica gel chromatography and twice by automated RP-LC. 152 mg (33% yield) of the title compound were obtained.

MS-ESI⁺ (m/z): 466

¹H NMR (500 MHz, (CD₃OD; δ, ppm): 8.72 (m, 1H), 8.10 (d, 1H, J=7.40 Hz), 8.06 (m, 1H) 7.61 (m, 2H), 7.38 (m, 1H), 7.17-7.11 (m, 3H), 6.84 (m, 2H), 6.08 (d, 1H, J=15.79 Hz), 5.62 (m, 1H), 3.71 (m, 1H), 3.13 (m, 2H), 2.67 (s, 3H), 2.42-2.26 (m, 4H), 2.24 (s, 6H)

### Example 39

### Synthesis of (R)-2-methyl-4-bromobenzyl-1-suphonic acid(2-benzyloxy-1-[2-dimethylaminoethylcarbamoyl)-ethyl]amide (compound 73)

### Step I

Boc-D-Ser(Bzl)-OH (298 mg, 295.34 g/mol, 1.0 mmol, 1.0 eq) and unsym-dimethylethylenediamine (111 μl, 88.15 g/mol, 0.80 g/cm³, 1.0 mmol, 1.0 eq) were coupled according to the procedure described in Example 36, step I. The reaction product was purified by silica gel chromatography to obtain 176 mg (48% yield) of (R)-2-(N-Boc-amino)-3-benzyloxy-N′-(2-dimethylaminoethyl)propionamide.

### Step II

The Boc protection was removed from (R)-2-(N-Boc-amino)-3-benzyloxy-N′-(2-dimethylaminoethyl)propionamide (175 mg, 365.48 g/mol, 0.48 mmol) according to the procedure described in Example 3, step V. The reaction product was purified by silica gel chromatography to obtain (R)-2-amino-3-benzyloxy-N-(2-dimethylaminoethyl)propionamide in quantitative yield.

### Step III

(R)-2-amino-3-benzyloxy-N′-(2-dimethylaminoethyl)propionamide (61 mg, 379.38 g/mol, 0.16 mmol, 1.0 eq) was sulfonylated with 4-bromo-2-ethylbenzene-1-sulfonyl chloride (70.3 mg, 283.57 g/mol, 0.24 mmol, 1.5 eq.) according to the procedure described in Example 3, step IV. The reaction product was purified by preparative RP-HPLC-column to give 3.1 mg (4% yield) of the title compound in pure form.

MS-ESI⁺ (m/z): 514

¹H NMR (500 MHz, (CD₃OD; δ, ppm): 8.46 (s, br, 1H), 7.83 (m, 1H), 7.52 (d, 1H, J=2.09 Hz), 7.46 (dd, 1H, J=2.09 Hz, J=8.49 Hz), 7.34-7.26 (m, 3H), 7.19 (m, 2H), 4.36 (m, 2H), 3.89 (m, 1H), 3.60 (m, 1H), 3.51 (m, 1H), 3.40 (m, 2H), 3.08-2.83 (m, 4H), 2.66 (m, 6H), 1.23 (m, 3H)

### Example 40

### Synthesis of 4-methylnaphthalene-1-sulfonic acid (3-phenoxy-1-piperidin-4-yl-propyl)-amide (compound 74)

### Step I

3-N-Fmoc-amino-3-(4-N′-Boc-piperidinyl)propionic acid (215 mg, 494.58 g/mol, 0.44 mmol, 1 eq), TEA (78.5 μl, 101.19 g/mol, 0.73 g/cm³, 0.57 mmol, 1.3 eq), ethyl chloroformate (54.1 μl, 108.52 g/mol, 1.14 g/cm³, 0.57 mmol, 1.3 eq) and sodium borohydride (18.1 mg, 0.48 mmol, 1.1 eq) were allowed to react according to the procedure described in Example 3, step I. In this manner 190 mg (91% yield) of 3-N-Fmoc-amino-3-(N′-Boc-piperidin-4-yl)propan-1-ol were obtained in pure form.

### Step II

3-N-Fmoc-amino-3-(N′-Boc-piperidin-4-yl)-propan-1-ol (190 mg, 480.61 g/mol, 0.40 mmol, 1.0 eq.) was dissolved in dry THF together with phenol (59.6 mg, 94.11 g/mol, 0.59 mmol, 1.5 eq) and triphenylphosphine (156 mg, 262.29 g/mol, 0.59 mmol, 1.5 eq) before DEAD (92.4 μl, 174.16 g/mol, 1.12 g/cm³, 0.59 mmol, 1.5 eq) was added in a dropwise manner. After reacting overnight at 50° C., the reaction mixture was evaporated to dryness and the residue was purified by silica gel chromatography. The purification provided 3-phenoxy-1-(N-Boc-piperidin-4-yl)-N′-Fmoc-propyl-amine in quantitative yield (489 mg).

### Step III

Fmoc protection was removed by dissolving the 3-phenoxy-1-(N-Boc-piperidin-4-yl)-N′-Fmoc-propyl-amine (489 mg, 556.71 g/mol, 0.88 mmol) in 3 ml DMF containing 20 vol-% piperidine. After 20 minutes of stirring, the solvent and excess piperidine were evaporated and the residue was dissolved in 1 M HCl-solution. The acidic water phase was washed thrice with DCM and then made alkaline by adding aq. 5 M NaOH-solution before the product was extracted four times with DCM. The combined organic fractions were dried over Na₂SO₄ and the solvent evaporated to give 57.9 mg (20% yield) of 3-phenoxy-1-(N-Boc-piperidin-4-yl)propylamine in pure form.

### Step IV

3-phenoxy-1-(N-Boc-piperidin-4-yl)propylamine (57.9 mg, 334.46 g/mol, 0.17 mmol) was sulfonylated with 4-methyl-1-naphthalenesulfonyl chloride (62.5 mg, 240.71 g/mol, 0.26 mmol, 1.5 eq) according to the procedure described in Example 3, step IV. After silica gel chromatography purification 51 mg (55%) of 3-phenoxy-1-(N-Boc-piperidin-4-yl)propylamine were obtained in pure form.

### Step V

The Boc protection was removed by treating 3-phenoxy-1-(N-Boc-piperidin-4-yl)propylamine (50.8 mg, 538.71 g/mol, 0.09 mmol) with TFA according to the procedure described in Example 3, step V. Preparative RP-HPLC purification yielded 1.7 mg (4%) of the title compound in pure form.

MS-ESI⁺ (m/z): 439

¹H NMR (500 MHz, (CD₃OD; δ, ppm): 8.74 (m, 1H), 8.52 (s, 1H), 8.08 (m, 1H), 8.02 (d, 1H, J=7.41 Hz), 7.66 (m, 2H), 7.17 (m, 1H), 7.06 (m, 2H), 6.82 (m, 1H), 6.11 (m, 2H), 3.44-3.38 (m, 3H), 3.33-3.27 (m, 1H), 2.99-2.89 (m, 3H), 2.56 (s, 3H), 2.02-1.52 (m, 7H)

### Example 41

### Synthesis of (S)-4-Methylnaphthalene-1-sulfonic acid(1-allyloxymethyl-4-amino-butyl)-amide (compound 75)

### Step I

Boc-L-Ornithinol(Z) (331 mg, 352.43 g/mol, 0.94 mmol, 1.0 eq.) was treated with allyl bromide (567 mg, 120.97 g/mol, 4.70 mmol), silver(I) oxide (1.09 g, 231.74 g/mol, 4.70 mmol, 5.0 eq.) and tertbutylammonium iodide (34.6 mg, 369.36 g/mol, 0.09 mmol, 0.1 eq.) in toluene according to the procedure described in Example 3, step II. After silica gel chromatography purification 236 mg (64% yield) of (S)-5-allyloxy-4-N-Boc-1-N′-Z-pentane-1,4-diamine were obtained in pure form.

### Step II

The Boc protection was removed by treating (S)-5-allyloxy-4-N-Boc-1-N′-Z-pentane-1,4-diamine with TFA according to the procedure described in Example 3, step V. After washing, the organic phase was dried over Na₂SO₄ and evaporated to give 236 mg of (S)-5-allyloxy-1-N-Z-pentane-1,4-diamine.

### Step III

(S)-5-allyloxy-1-N-Z-pentane-1,4-diamine (236 mg, 292.38 g/mol, 0.81 mmol, 1.0 eq.) was sulfonylated with 4-methyl-1-naphthalenesulfonyl chloride (273 mg, 240.71 g/mol, 1.13 mmol, 1.4 eq.) according to Example 3, step IV. After silica gel chromatography purification 351 mg (87% yield) of (S)-4-methylnaphthalene-1-sulfonic acid [4-(N-Z-amino)-1-allyloxymethyl-butyl]-amide were obtained in pure form.

### Step IV

The Z-protection was removed by dissolving (S)-4-methylnaphthalene-1-sulfonic acid [4-(N-Z-amino)-1-allyloxymethyl-butyl]-amide (155 mg, 496.63 g/mol, 0.3 mmol, 1.0 eq.) in MeCN (1.5 ml), followed by the addition of iodotrimethylsilane (53.6 μl, 200.09 g/mol, 0.38 mmol, 1.3 eq.). After a 1 hour reaction time, a few drops of aq. 10% Na₂S₂O₃ were added and the reaction mixture was evaporated to dryness. The residue was purified by silica gel chromatography. After purification the product was dissolved in DCM and the organic phase was washed with solutions of sat. aq. NaHCO₃, sat aq. NaCl, and sat. aq. Na₂S₂O₃, pH adjusted to 12 with 5 M NaOH-solution. Finally, the organic phase was dried over Na₂SO₄ and evaporated to obtain 8.5 mg (9% yield) mg of the title compound.

MS-ESI⁺ (m/z): 363

¹H NMR (500 MHz, CD₃OD; δ, ppm): 8.73 (r, 1H), 8.55 (s, 1H), 8.19 (m, 1H), 8.14 (d, 1H, J=7.53 Hz), 7.69 (m, 2H), 7.46 (d, 1H, J=7.53 Hz), 5.48-5.39 (m, 1H), 4.91 (m, 2H), 3.43 (m, 2H), 3.37-3.23 (m, 1H), 3.00 (m, 1H), 2.86 (m, 1H), 2.78 (s, 3H), 2.62 (m, 2H), 1.57-1.40 (m, 4H)

### Example 42

### Synthesis of (S)-4-methylnaphthalene-1-sulfonic acid (4-amino-1-ethoxymethylbutyl)amide (compound 76)

### Step I

Boc-L-ornithinol(Z) (176 mg, 352.43 g/mol, 0.50 mmol, 1.0 eq.) was treated in toluene with ethyliodide (60 μl, 155.96 g/mol, 1.95 g/cm³, 0.75 mmol), silver(I) oxide (463 mg, 231.73 g/mol, 2.0 mmol, 4.0 eq.) and TBAI (18 mg, 369.36 g/mol, 0.05 mmol, 0.1 eq.) according to the procedure described in Example 3, step II. The reaction product was purified by silica gel chromatography to obtain 65 mg (34% yield) of (S)-5-ethoxy-4-N-Boc-1-N′-Z-pentane-1,4-diamine.

### Step II

The Boc protection was removed from (S)-5-ethoxy-4-N-Boc-1-N′-Z-pentane-1,4-diamine (65 mg, 380.49 g/mol, 0.17 mmol) according to the procedure described in Example 34, step IV. The thus obtained (S)-5-ethoxy-1-N-Z-pentane-1,4-diamine trifluoroacetic acid salt was used without further purification in the next reaction step.

### Step III

(S)-5-Ethoxy-1-N-Z-pentane-1,4-diamine trifluoroacetic acid salt (0.17 mmol) was sulfonylated with 4-methyl-1-naphthalenensulfonyl chloride (82 mg, 240.71 g/mol, 0.34 mmol) according to the procedure described in Example 3, step IV. The reaction product was purified by silica gel chromatography to obtain 75 mg (91% yield) of (S)-4-methylnaphthalene-1-sulfonic acid [4-(Z-amino)-1-ethoxymethylbutyl]amide in pure form.

### Step IV

The Z protection was removed by dissolving (S)-4-methylnaphthalene-1-sulfonic acid [4-(Z-amino)-1-ethoxymethylbutyl]amide (75 mg, 484.62 g/mol, 0.16 mmol, 1.0 eq.) in MeCN (2 ml) followed by the addition of iodotrimethylsilane (55 μl, 200.09 g/mol, 1.4 g/cm³, 0.388 mmol, 2.5 eq.). After overnight stirring at room temperature, the reaction mixture was evaporated to dryness and the residue was dissolved in DCM. The organic phase was washed with 10% aq. Na₂S₂O₃, sat. aq. NaHCO₃ and brine before it was dried over Na₂SO₄ and evaporated. The reaction product was purified by silica gel chromatography to obtain 18 mg (32% yield) of the title compound.

MS-ESI⁺ (m/z): 351

¹H NMR (500 MHz, CD₃OD; δ, ppm): 8.72 (m, 1H), 8.19 (m, 1H), 8.15 (m, 1H), 7.70 (m, 2H), 7.46 (m, 1H), 2.95-2.80 (m, 4H), 2.87-2.82 (m, 1H), 2.80-2.75 (m, 2H), 2.78 (s, 3H), 1.82-1.64 (m, 2H), 1.62-1.46 (m, 2H), 0.72 (t, 3H, J=7.05 Hz, J=14.11 Hz)

### Example 43

### Synthesis of 4-methylnaphthalene-1-sulfonic acid (4-amino-1-cyclohexylaminomethyl-butyl)-amide formic acid salt (compound 77)

### Step I

Fmoc-Orn(Boc)-OH (459 mg, 454.52 g/mol, 1.01 mmol, 1.0 eq.), DIC (158 μl, 126.20 g/mol, 0.81 g/cm³, 1.01 mmol, 1.2 eq) and HOBt (137 mg, 135.12 g/mol, 1.01 mmol, 1.0 eq) were dissolved in DMF/DCM (1/1, 3 ml, dry). After 15 minutes stirring, cyclohexylamine (140 μl, 99.17 g/mol, 0.87 g/cm³, 1.21 mmol, 1.2 eq) was added to the reaction mixture and the stirring was continued overnight at room temperature. The reaction mixture was evaporated to dryness and the residue was dissolved in DCM. The organic phase was washed thrice with water and dried over Na₂SO₄. The thus obtained (S)—N-cyclohexyl-5-(N′-Boc-amino)-2-(N″-Fmoc-amino)pentanamide (541 mg) was used without further purification in the next reaction step.

### Step II

The Fmoc protection was removed from (S)-5-(N′-Boc)-2-(N″-Fmoc-amino) N-cyclohexyl pentanamide as described in Example 40, step II. In this manner 328 mg of (S)-2-amino-5-(N-Boc-amino)-N′-cyclohexylpentanamide were obtained in quantitative yield.

### Step III

(S)-2-amino-5-(N-Boc-amino)-N′-cyclohexylpentanamide (328 mg, 313.44 g/mol, 1.05 mmol, 1.0 eq.) was sulfonylated with 4-methyl-1-naphthalenesulfonyl chloride (352 mg, 240.71 g/mol, 1.50 mmol, 1.5 eq) according to the procedure described Example 3, step IV. Silica gel chromatography purification yielded 324 mg (60% yield) of (S)-4-methylnaphthalene-1-sulfonic acid [4-(N-Boc-amino)-1-cyclohexylcarbamoyl-butyl]amide.

### Step IV

The Boc protection was removed by treating (S)-4-methylnaphthalene-1-sulfonic acid [4-(N-Boc-amino)-1-cyclohexylcarbamoyl-butyl]amide (328 mg, 517.7 g/mol, 0.63 mmol) with TFA according to the procedure described in Example 3, step V. Silica gel chromatography purification yielded 211 mg (80% yield) of (S)-4-methylnaphthalene-1-sulfonic acid (4-amino-1-cyclohexylcarbamoyl-butyl)amide.

### Step V

The peptide bond of (S)-4-methylnaphthalene-1-sulfonic acid (4-amino-1-cyclohexylcarbamoyl-butyl)amide (209 mg, 417.57 g/mol, 0.50 mmol) was reduced according to the procedure described in Example 16, step VI. The crude product was purified by semi-preparative RP-HPLC to give 70 mg (35%) of the title compound in form of its formic acid salt.

MS-ESI⁺ (m/z): 404

¹H NMR (500 MHz, CD₃OD; δ, ppm): 8.74 (m, 1H), 8.25-8.19 (m, 2H), 7.78-7.71 (m, 2H), 7.52 (m, 1H), 3.47-3.42 (m, 1H), 2.86-2.69 (m, 2H), 2.80 (s, 3H), 2.46 (m, 2H), 1.77 (m, 2H), 1.77 (m, 4H), 1.66 (m, 1H), 1.45-1.36 (m, 2H), 1.33-1.09 (m, 8H)

### Example 44

### Synthesis of (S)-4-fluoronaphthalene-1-sulfonic acid (4-amino-1-benzyloxymethylbutyl)amide (compound 78)

### Step I

Boc-L-Ornithinol(Z) (352 mg, 352.43 g/mol, 1.0 mmol, 1.0 eq.), benzyl bromide (714 μl, 171.04 g/mol, 1.44 g/cm³, 6.0 mmol, 6.0 eq.), silver(I)oxide (1.16 g, 231.73 g/mol, 5.0 mmol, 5.0 eq.) and TBAI (37 mg, 369.36 g/mol, 0.1 mmol, 0.1 eq.) were allowed to react according to the procedure described in Example 3, step II. The reaction product was purified by silica gel chromatography to obtain 267 mg (60% yield) of (S)-5-benzyloxy-4-N-Boc-1-N′-Z-pentane-1,4-diamine.

### Step II

The Boc protection was removed from (S)-5-benzyloxy-4-N-Boc-1-N′-Z-pentane-1,4-diamine (178 mg, 422.56 g/mol, 0.42 mmol) according to the procedure described in Example 34, step IV. The obtained (S)-5-benzyloxy-1-N-Z-pentane-1,4-diamine trifluoroacetic acid salt was used without further purification in the next reaction step.

### Step II

(S)-5-Benzyloxy-1-N-Z-pentane-1,4-diamine trifluoroacetic acid salt (0.42 mmol, 1.0 eq.) was sulfonylated with 4-fluoronaphthalenesulfonyl-1-chloride (134 mg, 244.67 g/mol, 0.55 mmol, 1.3 eq.) according to the procedure described in Example 3, step IV. The reaction product was purified by silica gel chromatography to obtain 214 mg (92% yield) of (S)-4-fluoronaphthalene-1-sulfonic acid [4-(N-Z-amino)-1-benzyloxymethylbutyl]amide.

### Step IV

The Z protection was removed from (S)-4-fluoronaphthalene-1-sulfonic acid [4-(N-Z-amino)-1-benzyloxymethylbutyl]amide (214 mg, 550.64 g/mol, 0.39 mmol) according to the procedure described in Example 41, step IV. The reaction product was purified by silica gel chromatography to obtain 83 mg (52% yield) of the title compound in pure form.

MS-ESI⁺ (m/z): 417

¹H NMR (500 MHz, CD₃OD; δ, ppm): 8.74 (d, 1H, J=8.62 Hz), 8.23 (m, 1H), 8.20 (d, 1H, J=8.10 Hz), 7.73 (m, 2H), 7.22 (m, 4H), 6.98 (m, 2H), 4.04 (m, 2H), 3.29-3.27 (m, 2H), 3.12 (m, 1H), 3.04 (m, 1H), 2.46 (t, 2H, J=7.15 Hz, J=14.30 Hz), 1.57-1.50 (m, 1H), 1.45-1.36 (m, 2H), 1.34-1.26 (m, 1H)

### Example 45

### Synthesis of (S)-4-methylnaphthalene-1-sulfonic acid [1-benzyloxymethyl-3-(4,5-dihydro-1H-imidazol-2-ylamino)propyl]amide formic acid salt (compound 79)

### Step I

Boc-Dab(Z)-OH.DCHA (1.07 g, 533.71 g/mol, 2.0 mmol) was reduced according to the procedure described in Example 3, step I. In this manner 568 mg (84% yield) of (S)-4-(N-Z-amino)-2-(N′-Boc-amino)butan-1-ol were obtained.

### Step II

(S)-4-(N-Z-amino)-2-(N′-Boc-amino)butan-1-ol (284 mg, 338.41 g/mol, 0.84 mmol, 1.0 eq.) was treated in toluene with benzylbromide (400 μl, 171.04 g/mol, 1.44 g/cm³, 3.36 mmol, 4.0 eq), silver(I) oxide (779 mg, 231.73 g/mol, 3.36 mmol, 4.0 eq.) and TBAI (31 mg, 369.36 g/mol, 0.084 mmol, 0.1 eq.) according to the procedure described in Example 3, step II. The reaction product was purified by silica gel chromatography to obtain 209 mg (58%, yield) of (S)-4-benzyloxy-3-N-Boc-1-N′-Z-butane-1,3-diamine.

### Step III

The Boc protection was removed from (S)-4-benzyloxy-3-N-Boc-1-N′-Z-butane-1,3-diamine (209 mg, 428.53 g/mol, 0.49 mmol) according to the procedure described in Example 34, step IV. The thus obtained (S)-4-benzyloxy-1-N-Z-butane-1,3-diamine trifluoroacetic acid salt was used without further purification in the next reaction step.

### Step IV

(S)-4-benzyloxy-1-N-Z-butane-1,3-diamine trifluoroacetic acid salt (0.49 mmol, 1.0 eq.) was sulfonylated with 4-methyl-1-naphthalenesulfonyl chloride (178 mg, 240.71 g/mol, 0.74 mmol, 1.5 eq.) according to the procedure described in Example 3, step IV. The reaction product was purified by silica gel chromatography to obtain 230 mg (88% yield) of (S)-4-methylnaphthalene-1-sulfonic acid [3-(N-Z-amino)-1-benzyloxymethylpropyl]amide.

### Step V

The Z-protection was removed from (S)-4-methylnaphthalene-1-sulfonic acid [3-(N-Z-amino)-1-benzyloxymethylpropyl]amide (230 mg, 532.66 g/mol, 0.43 mmol) with iodotrimethylsilane (123 μl, 200.09 g/mol, 1.4 g/cm³, 0.86 mmol, 2.0 eq.) according to the procedure described in Example 42, step IV. The reaction product was purified by silica gel chromatography to obtain 58 mg (34% yield) of (S)-4-methyl-naphthalene-1-sulfonic acid (3-amino-1-benzyloxymethylpropyl)-amide.

### Step VI

(S)-4-Methyl-naphthalene-1-sulfonic acid (3-amino-1-benzyloxymethylpropyl)-amide (29 mg, 398.53 g/mol, 0.073 mmol, 1.0 eq.) was dissolved in water before NaOH (29 mg, 40.08 g/mol, 0.73 mmol, 10.0 eq.) and 2-methylsulfanyl-4,5-dihydro-1H-imidazolinium iodide (prepared by mixing equimolar amounts of imidazolinethione and methyl iodide in THF at 25° C. for 2 h). The solvent was then evaporated and the product used without further purification. 179 mg, 244.81 g/mol, 0.73 mmol, 10.0 eq) were added. The reaction mixture was stirred overnight at 50-60° C. Some water was added and the reaction mixture was extracted thrice with DCM. The combined organic fractions were washed with 10% aq. Na₂S₂O₃. The water phase was then made alkaline with NaOH and extracted twice with DCM. The combined organic fractions were dried over Na₂SO₄ and evaporated to yield 40 mg of crude product, which was purified by semi-preparative RP-HPLC to obtain 6.7 mg (16% yield) of the title compound.

MS-ESI⁺ (m/z): 467

¹H NMR (500 MHz, CD₃OD; δ, ppm); 8.70 (m, 1H), 8.49 (s, br), 1H), 8.19 (m, 1H), 8.13 (d, 1H, J=7.47 Hz), 7.68 (m, 2H), 7.42 (d, 1H, J=7.47), 7.68 (m, 2H), 7.42 (d, 1H, J=7.47), 7.20 (m, 3H), 6.92 (m, 2H), 3.92 (m, 2H), 3.67 (s, 4H), 3.39 (m, 1H), 3.24 (m, 2H), 2.98 (m, 1H), 2.88 (m, 1H), 2.76 (s, 3H), 1.83-1.76 (m, 1H), 1.72-1.64 (m, 1H)

### Example 46

### Synthesis of (S)-4-methyl-naphthalene-1-sulfonic acid {1-[(2-amino-ethylamino)methyl]-2-benzyloxy-ethyl]-amide (compound 65)

### Step I

Boc-D-Ser(Bzl)-OH (301 mg, 295.34 g/mol, 1 mmol, 1.0 eq.) was coupled with ethylenediamine (170 μl, 60.10 g/mol, 0.90 g/cm³, 2.5 mmol, 2.5 eq.) according to the procedure described in Example 35, step I. The reaction product was purified by silica gel chromatography to obtain 50 mg (15% yield) of (R)-2-(N-Boc-amino)-3-benzyloxy-N′-(2-ethylamino)propionamide.

### Step II

(R)-2-(N-Boc-amino)-N′-(2-ethylamino)propionamide (50 mg, 337.42 g/mol, 0.15 mmol, 1.0 eq.) was protected with 9-fluorenylmethylchloro-formate (44 mg, 258.70 g/mol, 0.16 mmol, 1.1 eq) according to the procedure described in Example Example 36, step II. The reaction product was purified by silica gel chromatography to obtain 55 mg (66% yield) of (R)-2-(N-Boc-amino)-3-benzyloxy-N′-(2-N″-Fmoc-aminoethyl)propionamide.

### Step III

The Boc protection was removed from (R)-2-(N-Boc-amino)-3-benzyloxy-N′-(2-N″-Fmoc-aminoethyl)propionamide (55 mg, 559.66 g/mol, 98 μmol) according to the procedure in Example 3, step V. The (R)-2-amino-3-benzyloxy-N-(2-N′-Fmoc-aminoethyl)propionamide was then used without further purification for step IV.

### Step IV

(R)-2-Amino-3-benzyloxy-N-(2-N′-Fmoc-aminoethyl)propionamide (45 mg (theoretical), 459.55 g/mol, 98 μmol, 1.0 eq.) was sulfonylated with 4-methyl-1-naphthalenesulfonyl chloride (44.4 mg, 240.71 g/mol, 184 μmol, 1.8 eq) according to the procedure described in Example 3, step IV. The reaction product was purified by silica gel chromatography to obtain 47 mg (72% yield) of (R)-4-methyl-naphthalene-1-sulfonic acid [2-benzyloxy-1-(2-N-Fmoc-aminoethylcarbamoyl)ethyl]amide.

### Step V

The Fmoc protection was removed from (R)-4-methylnaphthalene-1-sulfonic acid [2-benzyloxy-1-(2-N-Fmoc-aminoethylcarbamoyl)ethyl]amide (46 mg, 663.79 g/mol, 70 μmol) according to the procedure described in Example 2, step II. The reaction product was purified by silica gel chromatography to obtain 21 mg (69% yield) of (R)-4-methyl-naphthalene-1-sulfonic acid [2-benzyloxy-1-(2-aminoethylcarbamoyl)ethyl]amide.

### Step VI

(R)-4-Methyl-naphthalene-1-sulfonic acid [2-benzyloxy-1-(2-aminoethylcarbamoyl)ethyl]amide (18.4 mg, 441.55 g/mol, 42 μmol, 1.0 eq.) was treated with BTHF (375 μl, 1.0 M, 9.0 eq.) according to the procedure described in Example 2, step V. The reaction product was purified by preparative RP-HPLC-chromatography to obtain 1.7 mg (10% yield) of the title compound.

MS-ESI⁺ (m/z): 428

¹H NMR (500 MHz, CD₃OD; δ, ppm): 8.71 (m, 1H), 8.36 (s, (br), 1H), 8.19 (m, 1H), 8.16 (d, 1H, J=7.50), 7.68 (m, 2H), 7.43 (d, 1H, J=7.50), 7.21 (m, 3H), 6.96 (m, 2H), 3.98 (m, 1H), 3.62-3.40 (m, 2H), 3.10-3.07 (m, 1H), 3.02-2.97 (m, 1H), 2.92-2.80 (m, 2H), 2.77 (m, 3H), 2.72-2.64 (m, 3H), 1.83-1.56 (m, 1H)

### Example 47

### Synthesis of (S)-4-methylnaphthalene-1-sulfonic acid (4-amino-1-isobutoxymethylbutyl)amide (compound 80)

### Step I

Boc-L-Ornithinol(Z) (176 mg, 352.43 g/mol, 0.50 mmol, 1.0 eq.), 1-iodo-2-methylpropane (2.72 ml, 184.01 g/mol, 1.42 g/cm³, 21 mmol, 42 eq.), silver(I) oxide (1.39 g, 231.73 g/mol, 6.0 mmol, 12 eq.) and TBAI (92 mg, 369.63 g/mol, 0.25 mmol, 0.5 eq.) were allowed to react according to the procedure described in Example 3, step II. The reaction product was first purified by automated RP-LC and after that by silica gel chromatography to obtain 72 mg (35% yield) of (S)-5-isobutoxy-4-N-Boc-1-N′-Z-pentane-1,4-diamine.

### Step II

The Boc protection was removed from (S)-5-isobutoxy-4-N-Boc-1-N′-Z-pentane-1,4-diamine (71.6 mg, 408.54 g/mol, 0.175 mmol) according to the procedure described in Example 34, step IV. The thus obtained (S)-5-isobutoxy-1-N-Z-pentane-1,4-diamine trifluoroacetic acid salt was used without further purification in the next reaction step.

### Step III

(S)-5-isobutoxy-1-N-Z-pentane-1,4-diamine trifluoroacetic acid salt (0.175 mmol, 1.0 eq.) was sulfonylated with 4-methyl-1-naphtahalenesulfonyl chloride (63 mg, 240.71 g/mol, 0.263 mmol, 1.5 eq.) according to the procedure described in Example 3, step IV. The reaction product was purified by silica gel chromatography to obtain (S)-4-methylnaphthalene-1-sulfonic acid [4-(N-Z-amino)-1-isobutoxymethylbutylamide 77 mg (86% yield) in pure form.

### Step IV

The Z protection was removed from (S)-4-methylnaphthalene-1-sulfonic acid [4-(N-Z-amino)-1-isobutoxymethylbutyl]amide (76.8 mg, 512.67 g/mol, 0.15 mmol) with iodotrimethylsilane (32 μl, 200.09 g/mol, 1.4 g/cm³, 0.22 mmol, 1.5 eq.) according to the procedure described in Example 41, step IV, except that the solvent for the reaction was DCM. The reaction product was purified twice by silica gel chromatography to obtain 11 mg (20% yield) of the title compound.

MS-ESI⁺ (m/z): 379

¹H NMR (500 MHz, CD₃OD; δ, ppm): 8.74 (m, 1H), 8.20 (m, 1H), 8.15 (m, 1H), 7.68 (m, 2H), 7.46 (m, 1H), 3.22 (m, 1H), 3.07-2.92 (m, 2H), 2.78-2.72 (m, 4H), 2.64 (m, 1H), 2.45 (m, 2H), 1.51-1.28 (m, 5H), 0.66 (m, 6H)

### Example 48

### Synthesis of 4-methyl-naphthalene-1-sulfonic acid (3-amino-1-cyclo-hexylaminomethyl-propyl)-amide (compound 81)

### Step I

Fmoc-Dab(Boc)-OH (261 mg, 440.49 g/mol, 0.59 mmol, 1.0 eq.), DIC (92 μl, 126.20 g/mol, 0.81 g/cm³, 0.59 mmol, 1.0 eq.), HOBt (81.4 mg, 135.12 g/mol, 0.59 mmol, 1.0 eq.) were dissolved in DMF/DCM (1/1, 3 ml). After 5 minutes of stirring, cyclohexylamine (81 μl, 0.87 g/cm³, 99.18 g/mol, 0.71 mmol, 1.2 eq.) was added to the reaction mixture and the stirring was continued overnight at room temperature. The reaction mixture was evaporated to dryness and the residue was dissolved in DCM. The organic phase was washed thrice with water and once with brine before it was dried over Na₂SO₄ and evaporated to obtain (S)—N-cyclohexyl-4-(N′-Boc-amino)-2-(N″-Fmoc-amino)butyramide.

### Step II

The Fmoc protection was removed from (S)—N-cyclohexyl-4-(N′-Boc-amino)-2-(N″-Fmoc-amino)butyramide (0.59 mmol) according to the procedure described in Example 40, step II. The thus obtained (S)-2-amino-4-(N-Boc-amino)-1-cyclohexyl-butyramide was used without further purification in the next reaction step.

### Step III

(S)-2-Amino-4-(N-Boc-amino)-1-cyclohexyl-butyramide (0.59 mmol) was sulfonylated with 4-methyl-1-naphthalenesulfonyl chloride (213 mg, 240.71 g/mol, 0.89 mmol, 1.5 eq.) according to the procedure described in Example 1, step IV. The reaction product was purified by silica gel chromatography to obtain 198 mg (67% yield) of (S)-4-methylnaphthalene-1-sulfonic acid[3-(N-Boc-amino)-1-cyclohexylcarbamoyl-propyl]amide.

### Step IV

The Boc protection was removed from (S)-4-methylnaphthalene-1-sulfonic acid[3-(N-Boc-amino)-1-cyclohexylcarbamoyl-propyl]amide (198 mg, 503.67 g/mol, 0.39 mmol) according to the procedure described in Example 3, step V. The reaction product was purified by silica gel chromatography to obtain 124 mg (79% yield) of (S)-4-methylnaphthalene-1-sulfonic acid(3-amino-1-cyclohexylcarbamoyl-propyl)amide.

### Step V

(S)-4-methylnaphthalene-1-sulfonic acid(3-amino-1-cyclohexyl-carbamoylpropyl)amide (41.3 mg, 403.55 g/mol, 0.10 mmol, 1.0 eq.) was dissolved in THF and the solution was flushed with argon. BTHF (900 μl, 1.0 M, 0.90 mmol, 9.0 eq.) was added and the reaction mixture was refluxed for 4 hours. The reaction mixture then was quenched by the addition of methanol (2 ml) before it was evaporated to dryness. 1 ml of acetic acid and 1 ml of water were added and the mixture was refluxed for 30 minutes before it was again evaporated to dryness. The residue was dissolved in DCM and washed with sat. aq. NaHCO₃-solution. The organic phase was then dried over Na₂SO₄ before the solvent was evaporated. The reaction product was purified by silica gel chromatography to obtain 20 mg (51% yield) of the title compound in pure form.

MS-ESI⁺ (m/z): 390

¹H NMR (500 MHz, CD₃OD; δ, ppm): 8.74 (m, 1H), 8.21 (m, 2H), 7.71 (m, 2H), 7.48 (m, 1H), 3.29-3.26 (m, 2H), 2.78 (s, 3H), 2.64-2.53 (m, 2H), 2.32-2.24 (m, 2H), 1.80-1.73 (m, 1H), 1.57-1.45 (m, 5H), 1.30 (m, 1H), 1.16 (m, 1H), 1.03-0.93 (m, 2H), 0.52-0.45 (m, 2H)

### Example 49

### Synthesis of (S)-4-methylnaphthalene-1-sulfonic acid (4-amino-1-benzyloxymethylbutyl)methylamide (compound 82)

### Step I

Fmoc-Ornithinol(Boc)-OH (909 mg, 454.5 g/mol, 2.0 mmol, 1.0 eq.) was dissolved in methanol (28 ml). DCC (495 mg, 206.33 g/mol, 2.4 mmol, 1.2 eq.) and DMAP (24 mg, 122.17 g/mol, 0.2 mmol, 0.2 eq.) were added and the reaction mixture was stirred overnight at room temperature before it was evaporated to dryness and the residue was dissolved in DCM The formed precipitate was filtered off and the filtrate was evaporated to dryness. The reaction product was purified by silica gel chromatography to obtain 480 mg (53% yield) of (S)-5-N-Boc-2-N′-(Fmoc)-2,5-diaminopentanoic acid methyl ester.

### Step II

The Fmoc protection was removed from (S)-5-N-Boc-2-N′-(Fmoc)-2,5-diaminopentanoic acid methyl ester (480 mg, 468.55 g/mol, 1.024 mmol) according to the procedure described in Example 3, step III. The thus obtained (S)-5-N-Boc-2,5-diaminopentanoic acid methyl ester was used in the next reaction step without further purification.

### Step III

(S)-5-N-Boc-2,5-diaminopentanoic acid methyl ester (260 mg, 246.31 g/mol, 1.024 mmol, 1.0 eq.) was sulfonylated with 4-methyl-1-naphthalenesulfonyl chloride (555 mg, 240.71 g/mol, 2.304 mmol, 1.8 eq.) according to the procedure described in Example 3, step IV. The reaction product was purified by silica gel chromatography to obtain 183 mg (39% yield) of (S)-5-N-Boc-amino-2-(4-methylnaphthalene-1-sulfonylamino)pentanoic acid methyl ester.

### Step IV

(S)-5-N-Boc-amino-2-(4-methylnaphthalene-1-sulfonylamino)pentanoic acid methyl ester (183 mg, 450.56 g/mol, 0.406 mmol, 1.0 eq.) was dissolved in DMF (4 ml). DBU (182 μl, 152.24 g/mol, 1.018 g/cm³, 1.218 mmol, 3.0 eq.) and dimethyl sulfate (173 μl, 126.13 g/mol, 1.33 g/cm3, 4.5 eq.) were then added at 0° C. under argon to the reaction mixture. After a reaction time of 4 h at 0° C., the reaction mixture was evaporated to dryness and the reaction product was purified by silica gel chromatography to obtain 125 mg (66% yield) of (S)—N-5-Boc-amino-2-[methyl-(4-methylnaphthalene-1-sulfonyl)amino]pentanoic acid methyl ester.

### Step V

Sodium borohydride (48.9 mg, 37.82 g/mol, 1.29 mmol, 5.0 eq.) was dissolved in THF/water (4:1, 2.5 ml) and cooled to 0° C. (S)—N-5-Boc-amino-2-[methyl-(4-methylnaphthalene-1-sulfonyl)amino]pentanoic acid methyl ester (125 mg, 464.59 g/mol, 0.269 mmol, 1.0 eq.) was dissolved in THF and added dropwise to the NaBH₄-solution. When bubbling stopped, the cooling bath was removed and the reaction mixture was allowed to warm up to room temperature. After a reaction time of 6 h the reaction mixture was evaporated to dryness. The residue was dissolved in water and extracted with ethyl acetate. The organic phase was then washed with 10% aq. citric acid-solution, saturated aq. NaHCO₃-solution and brine. The water phase was made slightly acid with a concentrated HCl-solution and extracted with ethyl acetate. The organic phase was washed with saturated aq. NaHCO₃-solution and brine. The combined organic phases were then dried over Na₂SO₄ and evaporated to dryness. The thus obtained (S)-4-methylnaphthalene-1-sulfonic acid (4-N-Boc-amino-1-hydroxymethylbutyl)methylamide was used in the next reaction step without further purification.

### Step VI

(S)-4-methylnaphthalene-1-sulfonic acid (4-N-Boc-amino-1-hydroxymethylbutyl)methylamide (117 mg, 436.57 g/mol, 0.269 mmol, 1.0 eq.) was treated with benzyl bromide (128 μl, 171.04 g/mol, 1.438 g/cm³, 1.076 mmol, 4.0 eq.), silver(I) oxide (249.3 mg, 231.74 g/mol, 1.076 mmol, 4.0 eq.) and tert-butylammonium iodide (10 mg, 369.36 g/mol, 0.027 mmol, 0.1 eq.) in toluene according to the procedure described in Example 0.3, step II, except that the reaction time was 2 d. The thus obtained crude (S)-4-Methyl-naphthalene-1-sulfonic acid (4-N-Boc-amino-1-benzyloxymethylbutyl)methyl-amide (152 mg) was used in the next reaction step without further purification.

### Step VII

The boc protection was removed from (S)-4-methylnaphthalene-1-sulfonic acid (4-N-Boc-amino-1-benzyloxymethylbutyl)methylamide (152 mg, 526.70 g/mol, 0.288 mmol) according to the procedure described in Example 3, step V. The reaction product was purified by silica gel chromatography to obtain 25 mg (21% yield) of the title compound in pure form.

MS-ESI⁺ (m/z): 427

¹H NMR (500 MHz, CD₃OD; δ, ppm): 8.72 (m, 1H), 8.15 (m, 2H), 7.63 (m, 2H), 7.37 (m, 1H), 7.21 (m, 3H), 6.98 (m, 2H), 4.10 (m, 3H), 3.25 (m, 2H), 2.99-2.84 (m, 2H), 2.78 (s, 3H), 2.74 (s, 3H), 1.74-1.66 (m, 2H), 1.64-1.60 (m, 2H)

### Example 50

### Synthesis of (S)-naphthalene-1-sulfonic acid (4-amino-1-benzyloxymethylbutyl)amide (compound 83)

The compound was synthesized according to the procedure described in Example 44, except that instead of 4-fluoronaphthalenesulfonyl chloride naphthalene-1-sulfonyl chloride was used. In this manner, the title compound was obtained in pure form with 10% overall yield.

MS-ESI⁺ (m/z): 399

¹H NMR (500 MHz, CD₃OD; δ, ppm): 8.72 (m, 1H), 8.24 (dd, 1H), 8.13 (m, 1H), 8.01 (m, 1H), 7.69-7.60 (m, 2H), 7.54 (m, 1H), 7.21 (m, 3H), 6.99 (m, 2H), 4.00 (m, 2H), 3.11 (m, 1H), 2.99 (m, 1H), 2.52 (m, 2H), 1.57-1.29 (m, 5H)

### Example 51

### Synthesis of (S)-4-methylnaphthalene-1-sulfonic acid [2-(1H-imidazol-4-yl)-1-phenoxymethylethyl]amide (compound 84)

### Step I

Boc-L-Histidinol(Tos) (791 mg, 395.48 g/mol, 2.0 mmol, 1.0 eq) was treated with phenol (226 mg, 94.11 g/mol, 2.4 mmol, 1.2 eq.), triphenylphosphine (629 mg, 262.29 g/mol, 2.4 mmol, 1.2 eq.) and DEAD (378 μl, 174.16 g/mol, 1.11 g/cm³, 2.4 mmol, 1.2 eq.) according to the procedure described in Example 13, step I. The reaction product was purified by silica gel chromatography to obtain 494 mg (52% yield) of (S)-3-phenoxy-2-N-Boc-1-[(1-Tos)imidazol-4-yl]propane-2-amine.

### Step II

The Boc protection was removed from (S)-3-phenoxy-2-N-Boc-1-[(1-Tos)imidazol-4-yl]propane-2-amine (494 mg, 471.58 g/mol, 1.05 mmol) according to the procedure described in Example 34, step. IV. The thus obtained (S)-3-phenoxy-1-[(1-Tos)imidazol-4-yl]propane-2-amine trifluoroacetic acid salt was used without further purification in the next reaction step.

### Step III

(S)-3-phenoxy-1-[(1-Tos)imidazol-4-yl]propane-2-amine trifluoroacetic acid salt (1.05 mmol, 1.0 eq.) was sulfonylated with 4-methyl-1-naphthalenesulfonyl chloride (378 mg, 240.71 g/mol, 1.57 mmol, 1.5 eq.) according to the procedure described in Example 3, step IV. The tosyl protection group was cleaved during the sulfonylation. The reaction product was purified by silica gel chromatography to obtain 100 mg (23% yield) of the title compound.

MS-ESI⁺ (m/z): 421

### Example 52

### Synthesis of (S)-4-methylnaphthalene-1-sulfonic acid {4-amino-1-[(benzylmethylamino)methyl]butyl}amide (compound 85)

### Step I

Boc-L-Ornithinol(Z) (176 mg, 352.43 g/mol, 0.50 mmol, 1.0 eq.) was dissolved in DCM under argon. The solution was cooled to 0° C. and triflic anhydride (93 μl, 282.13 g/mol, 1.68 g/cm³, 0.55 mmol, 1.1 eq.) was added. The mixture was stirred for 10 minutes before 2,6-lutidine (70 μl, 107.15 g/mol, 0.92 g/cm³, 0.60 mmol, 1.2 eq.) was added. The mixture was stirred another 10 minutes before a pre-mixed solution of N-methylbenzylamine (97 μl, 121.18 g/mol, 0.94 g/cm³, 0.75 mmol, 1.5 eq.) and TEA (152 μl, 101.19 g/mol, 0.73 g/cm³, 1.1 mmol, 2.2 eq.) in DCM was added. Stirring was continued for 30 minutes at 0° C. and thereafter overnight at room temperature. The reaction mixture was diluted with DCM and washed with a sat. aq. NaHCO₃-solution. The organic phase was then dried over Na₂SO₄ and evaporated. The reaction product was purified by silica gel chromatography to obtain 24 mg (11% yield) of (S)-5-benzylmethylamino-4-N-Boc-1-N′-Z-pentane-1,4-diamine in pure form.

### Step II

The Boc protection was removed from (S)-5-benzylmethylamino-4-N-Boc-1-N′-Z-pentane-1,4-diamine (24 mg, 455.60 g/mol, 0.053 mmol, 1.0 eq.) according to the procedure described in Example 34, step IV, except that the reaction time was 40 minutes. The thus obtained (S)-5-benzylmethylamino-1-N-Z-pentane-1,4-diamine trifluoroacetic acid salt was used without further purification in the next reaction step.

### Step II

(S)-5-benzylmethylamino-1-N-Z-pentane-1,4-diamine trifluoroacetic acid salt (0.053 mmol, 1.0 eq.) was sulfonylated with 4-methyl-1-naphthalenesulfonyl chloride (19 mg, 240.71 g/mol, 0.079 mmol, 1.5 eq.) according to the procedure described in Example 3, step IV. The reaction product was purified by silica gel chromatography to obtain 3.3 mg (11% yield) of (S)-4-methylnaphthalene-1-sulfonic acid [4-(N-Z-amino)-1-benzylmethylaminomethylbutyl]amide.

### Step IV

The Z protection was removed from (S)-4-methylnaphthalene-1-sulfonic acid [4-(N-Z-amino)-1-benzylmethylaminomethylbutyl]amide. (3.3 mg, 559.73 g/mol, 0.006 mmol, 1.0 eq.) according to the procedure described in Example 42, step IV, except that 42 eq. iodotrimethylsilane were used and the reaction time was 1 week. The reaction product was purified by silica gel chromatography to obtain 0.8 mg (32% yield) of title compound in pure form.

MS-ESI⁺ (m/z): 426

### Example 53

### Synthesis of (R)-4-methylnaphthalene-1-sulfonic acid (N-aminomethyl-carbamoyl-2-benzyloxyethyl)amide (compound 86)

### Step I

Boc-D-Ser(Bzl)-OH (295 mg, 295.34 g/mol, 1.0 mmol, 1.0 eq.) was treated with DCC (248 mg, 206.33 g/mol, 1.2 mmol, 1.2 eq.), HOBt (135 mg, 135.12 g/mol, 1.0 mmol, 1.0 eq.), glycinamide hydrochloride (133 mg, 110.54 g/mol, 1.2 mmol, 1.2 eq.) and TEA (166 μl, 101.19 g/mol, 0.73 g/cm³, 1.2 mmol, 1.2 eq.) in DMF/DCM (1/1, 4 ml, dry) according to the procedure described in Example 2, step I. The reaction product was first purified by silica gel chromatography and after that by automated RP-LC to obtain 289 mg (82% yield) of (R)—N-carbamoylmethyl-3-benzyloxy-2-N′-Boc-aminopropionamide in pure form.

### Step II

The Boc protection was removed from (R)—N-carbamoylmethyl-3-benzyloxy-2-N′-Boc-aminopropionamide (289 mg, 351.40 g/mol, 0.82 mmol) according to the procedure described in Example 34, step IV, except that the reaction time was 3 hours. The thus obtained (R)—N-carbamoylmethyl-3-benzyloxy-2-aminopropionamide trifluoroacetic acid salt was used without further purification in the next reaction step.

### Step III

(R)—N-carbamoylmethyl-3-benzyloxy-2-aminopropionamide trifluoroacetic acid salt (0.82 mmol, 1.0 eq.) was sulfonylated with 4-methyl-1-naphtahalenesulfonyl chloride (355 mg, 240.71 g/mol, 1.47 mmol, 1.8 eq.) according to the procedure described in Example 3, step IV. The reaction product was purified by silica gel chromatography to obtain 120 mg (32% yield) of (R)-4-methylnaphthalene-1-sulfonic acid (N-carbamoylmethylcarbamoyl-2-benzyloxyethyl)amide.

### Step IV

PIFA (57 mg, 430.04 g/mol, 0.13 mmol, 1.0 eq.) was dissolved in MeCN/H₂O (1/1, 1.4 ml), (R)-4-methylnaphthalene-1-sulfonic acid (N-carbamoylmethylcarbamoyl-2-benzyloxyethyl)amide (60 mg, 455.53 g/mol, 0.13 mmol, 1.0 eq.) was added and the reaction mixture was stirred overnight at room temperature. The reaction mixture was evaporated to dryness and the residue was dissolved in water. The water phase was first made acidic with HCl and washed twice with ethyl acetate before it was made alkaline with a sat. aq. NaHCO₃-solution and extracted thrice with DCM. The combined organic fractions were dried over Na₂SO₄ and evaporated. 30 mg (54% yield) of the title compound in pure form was obtained.

MS-ESI⁺ (m/z): 428

### Example 54

### Synthesis of (S)-4-methylnaphthalene-1-sulfonic acid [4-amino-1-(thiophen-3-yloxymethyl)-butyl]-amide (compound 87)

### Step I

Potassium-3-thiophenefluoroborate (252 mg, 190.04 g/mol, 1.32 mmol, 2.0 eq.) was dissolved in DCM, copper(II) acetate monohydrate (16.5 mg, 199.65 g/mol, 0.083 mmol, 0.1 eq) and DMAP (20.2 mg, 122.17 g/mol, 0.165 mmol, 0.2 eq.) were added and the mixture was stirred for 5 minutes before Boc-L-ornithinol(Z) (233 mg, 352.43 g/mol, 0.662 mmol, 1.0 eq.) was added and the stirring was continued overnight at room temperature. The reaction mixture was then filtered through celite and the filtrate was evaporated to dryness. The reaction product was purified by silica gel chromatography to obtain 72 mg (25% yield) of (S)-5-(thiophen-3-yloxy)-4-N-Boc-1-N′-Z-pentane-1,4-diamine.

### Step II

The Boc protection was removed from (S)-5-(thiophen-3-yloxy)-4-N-Boc-1-N′-Z-pentane-1,4-diamine (72.3 mg, 434.56 g/mol, 0.166 mmol) according to the procedure described in Example 3, step V. The thus obtained (S)-5-(thiophen-3-yloxy)-1-N-Z-pentane-1,4-diamine was used without further purification in the next reaction step.

### Step III

(S)-5-(thiophen-3-yloxy)-1-N-Z-pentane-1,4-diamine (55.6 mg, 334.44 g/mol, 0.166 mmol, 1.0 eq.) was sulfonylated with 4-methyl-1-naphthalenesulfonyl chloride (52.1 mg, 240.71 g/mol, 0.216 mmol, 1.3 eq.) according to the procedure described in Example 3, step IV, except that the reaction mixture was stirred 2 days at 50° C. The reaction product was purified by silica gel chromatography to obtain 43 mg (48% yield) of (S)-4-methylnaphthalene-1-sulfonic acid [4-(N-Z-amino)-1-(thiophen-3-yloxymethyl)butyl]-amide.

### Step IV

The Z protection was removed from (S)-4-methylnaphthalene-1-sulfonic acid [4-(N-Z-amino)-1-(thiophen-3-yloxymethyl)-butyl]-amide (43.4 mg, 538.69 g/mol, 0.081 mmol) according to the procedure described in Example 1, step V. The reaction product was purified by automated RP-LC to obtain 8.9 mg (27% yield) of the title compound.

MS-ESI⁺ (m/z): 405

### Example 55

### Synthesis of (S)-4-methyl-naphthalene-1-sulfonic acid [4-amino-1-(1-phenyl-ethoxy methyl)-butyl]-amide formic acid salt (compound 88)

### Step I

Fmoc-Orn(Boc)-OH (400 mg, 454.5 g/mol, 0.88 mmol) was reduced according to the procedure described in Example 3, step I.

### Step II

(S)-5-(N-Boc-amino)-2-(N′-Fmoc-amino)-pentan-1-ol (179 mg, 440.54 g/mol, 0.405 mmol) was alkylated with 1-bromoethylbenzene (375 mg, 185.06 g/mol, 2.03 mmol, 5.0 eq.) according to the procedure described in Example 3, step II. The reaction product was purified by silica gel chromatography to obtain 67 mg (30% yield) of (S)-5-(1-phenyl-ethoxy)-4-N-Fmoc-1-N′-Boc-pentane-1,4-diamine.

### Step III

The Fmoc protection was removed from (S)-5-(1-phenyl-ethoxy)-4-N-Fmoc-1-N′-Boc-pentane-1,4-diamine (67 mg, 544.69 g/mol, 0.123 mmol) according to the procedure described in Example 3, step III. The reaction product was purified by silica gel chromatography to obtain 22 mg (56% yield) of (S)-5-(1-phenylethoxy)-1-N-Boc-pentane-1,4-diamine.

### Step IV

(S)-5-(1-phenyl-ethoxy)-1-N-Boc-pentane-1,4-diamine (12.9 mg, 322.45 g/mol, 0.04 mmol, 1.0 eq.) was sulfonylated with 4-methyl-1-naphthalenesulfonyl chloride (14.5 mg, 240.71 g/mol, 0.06 mmol, 1.0 eq.) according to the procedure described in Example 3, step IV. The reaction product was purified by RP-HPLC to obtain 5.1 mg (24% yield) of (S)-4-methylnaphthalene-1-sulfonic acid [4-(N-Boc-amino)-1-(1-phenyl-ethoxy-methyl)-butyl]-amide.

### Step IV

The Boc protection was removed by dissolving (S)-4-methylnaphthalene-1-sulfonic acid [4-(N-Boc-amino)-1-(1-phenyl-ethoxy methyl)-butyl]-amide (1.9 mg, 526.70 g/mol, 6.07 mmol) in 20 vol-% formic acid in DCM (1.5 ml). The reaction mixture was stirred 4 days at room temperature before it was co-evaporated three times with EtOH to remove residual formic acid. 2.1 mg of the title compound was obtained.

MS-ESI⁺ (m/z): 427

### Example 56

### Synthesis of (S)-4-methylnaphthalene-1-sulfonic acid (1-aminomethyl-3-benzyloxy-propyl)-amide (compound 989)

### Step I

Boc-O-benzyl-D-homoserine (300 mg, 309.3 g/mol, 0.97 mmol, 1.0 eq.) was reduced according to the procedure described in Example 3, step I. The thus obtained (S)-4-benzyloxy-2-(N-Boc-amino)-butan-1-ol 159 mg (55% yield) was used without further purification in the next reaction step.

### Step II

(S)-4-benzyloxy-2-(N-Boc-amino)butan-1-ol (159 mg, 295.38 g/mol, 0.54 mmol, 1.0 eq.), triphenylphosphine (184 mg, 262.29 g/mol, 0.702 mmol, 1.3 eq.) and phtalimide (103 mg, 147.13 g/mol, 0.702 mmol, 1.3 eq.) were dissolved in dry THF. The solution was cooled to 0° C. and DEAD (109 μl, 174.16 g/mol, 1.2 g/cm³, 0.702 mmol, 1.3 eq.) was added dropwise to the solution. The reaction mixture was allowed to warm up to room temperature and was for stirred 1 hour before it was evaporated to dryness. The reaction product was purified by silica gel chromatography to obtain 222 mg (97% yield) of (S)-4-benzyloxy-2-N-Boc-1-N′-phtaloyl-butane-1,2-diamine.

### Step III

The Boc protection was removed from (S)-4-benzyloxy-2-N-Boc-1-N′-phtaloyl-butane-1,2-diamine (222 mg, 424.50 g/mol, 0.52 mmol) according to the procedure described in Example 3, step V. The thus obtained (S)-4-benzyloxy-2-amino-1-N′-phtaloyl-butane-1,2-diamine 161 mg (95% yield) was used without further purification in the next reaction step.

### Step IV

(S)-4-benzyloxy-2-amino-1-N-phtaloyl-butane-1,2-diamine (161 mg, 324.38 g/mol, 0.49 mmol, 1.0 eq.) was sulfonylated with 4-methyl-1-naphthalenesulfonyl chloride (177 mg, 240.71 g/mol, 0.74 mmol, 1.5 eq.) according to the procedure described in Example 3, step IV, except that the reaction mixture was dissolved in DCM after it was evaporated to dryness, and it was washed with 5% aq. NaHCO₃ and brine. The organic phase was dried over Na₂SO₄ and evaporated. The reaction product was purified by RP-HPLC to obtain 28 mg (11% yield) of (S)-4-methylnaphthalene-1-sulfonic acid (1-N-phtaloyl-aminomethyl-3-benzyloxy-propyl)-amide.

### Step V

(S)-4-methylnaphthalene-1-sulfonic acid (1-N-phtaloyl-aminomethyl-3-benzyloxy-propyl)-amide (27.5 mg, 528.63 g/mol, 0.052 mmol, 1.0 eq.), hydrazinium hydroxide (11.3 μl, 50.06 g/mol, 1.03 g/cm³, 0.234 mmol, 4.5 eq.) and allyl alcohol (15.9 μl, 58.08 g/mol, 0.85 g/cm³, 0.234 mmol, 4.5 eq.) were dissolved in dioxane/DMF (1/1, 1.0 ml). The reaction mixture was first stirred overnight at room temperature and after that for 3 days at 40-50° C. After cooling to room temperature, the reaction mixture was diluted with EtOH. The precipitate that formed was filtered off and was washed with EtOH. The filtrate was then evaporated to dryness and the reaction product was purified by silica gel chromatography to obtain 6.6 mg (32% yield) of the title compound.

MS-ESI⁺ (m/z): 399

### Example 57

### Synthesis of (S)-4-methyl-naphthalene-1-sulfonic acid (3-benzyloxy-1-guanidinomethyl-propyl)-amide trifluoroacetic acid salt (compound 90)

### Step I

(S)-4-methylnaphthalene-1-sulfonic acid (1-aminomethyl-3-benzyloxy-propyl)-amide (Example 56, 15.2 mg, 398.53 g/mol, 0.038 mmol, 1.0 eq.) was guanidylated according to the procedure described in Example 4, step I, except that the reaction time was overnight at room temperature. The reaction mixture was diluted with DCM and washed with 10% aq. citric acid and brine. The organic phase was dried over Na₂SO₄ and evaporated. The reaction product was purified first by silica gel chromatography and after that with preparative TLC to obtain 0.4 mg (2% yield) of (S)-4-methylnaphthalene-1-sulfonic acid [3-benzyloxy-1-(1,3-N,N-Boc-guanidino) aminomethyl-propyl)amide,

### Step II

The Boc protections were removed from (S)-4-methylnaphthalene-1-sulfonic acid [3-benzyloxy-1-(1,3-N,N′-Boc-guanidino) aminomethyl-propyl)-amide according to the procedure described in Example 34, step IV to provide 0.4 mg of the title compound.

MS-ESI⁺ (m/z): 441

### Example 58

### Synthesis of (R)-4-methyl-naphthalene-1-sulfonic acid (1-benzyloxymethyl-2-guanidino-2-oxo-ethyl)-amide hydrochloric acid salt (compound 91)

### Step I

The Boc protection was removed from Boc-D-Ser(Bzl)-OH (200 mg, 293.34 g/mol, 0.68 mmol) according to the procedure described in Example 34, step IV. The thus obtained 2-(R)-2-amino-3-(benzyloxy)propionic acid trifluoroacetic acid salt was used without further purification in the next reaction step.

### Step II

(R)-2-amino-3-(benzyloxy)propionic acid trifluoroacetic acid salt (0.68 mmol) was sulfonylated with 4-methyl-1-naphthalenesulfonyl chloride (361 mg, 240.71 g/mol, 1.02 mmol, 1.5 eq., 1.0 eq.) according to the procedure described in Example 3, step IV. The reaction product was purified by silica gel chromatography to obtain 50 mg (18% yield) of (R)-3-benzyloxy-2-(4-methylnaphthalene-1-sulfonylamino)-propionic acid.

### Step III

(R)-3-Benzyloxy-2-(4-methyl-naphthalene-1-sulfonylamino) propionic acid (6.4 mg, 399.47 g/mol, 0.016 mmol, 1.0 eq.) was dissolved in DCM/DMF (1:1, 1 ml) before DCC (3.3 mg, 206.33 g/mol, 0.016 mmol, 1.0 eq.) and HOBt (2.4 mg, 153.12 g/mol, 0.016 mmol, 1.0 eq.) were added. 1,3-Bis(tert-butoxycarbonyl)guanidine (9.1 mg, 259.3 g/mol, 0.096 mmol, 6.0 eq.) was treated first with HCl, which removed only one boc group, and then with TEA (20 μl, 101.19 g/mol, 0.73 g/cm³, 0.144 mmol, 9.0 eq.) to liberate the HCl salt, which was added to the reaction mixture above. After stirring for one day at room temperature and then overnight at 30-40° C., the reaction mixture was evaporated to dryness and the residue was dissolved in DCM. The organic phase was washed with a 5% aq. NaHCO₃ solution and brine, dried over Na₂SO₄ and evaporated. The reaction product was purified by preparative TLC to obtain (R)-4-methyl-naphthalene-1-sulfonic acid [1-benzyloxymethyl-2-(1-N,-Boc-guanidino)-2-oxo-ethyl]-amide.

### Step IV

The Boc protection group were removed from (R)-4-methylnaphthalene-1-sulfonic acid [1-benzyloxymethyl-2-(1-N′-Boc-guanidino)-2-oxo-ethyl]-amide according to the procedure described in Example 34, step IV, except that the reaction mixture was stirred overnight at room temperature. The reaction mixture was then extracted with water, the water phase evaporated to dryness and the residue dissolved in 1 N HCl-solution before it was washed with EtOAc/hexane. The acidic water phase was evaporated to dryness to obtain 0.5 mg (7% yield) of the title compound in form of its HCl acid salt.

MS-ESI⁺ (m/z): 441

### Example 59

### Synthesis of (S)-4-bromo-naphthalene-1-sulfonic acid [4-amino-1-(isoquinolin-6-yloxymethyl)-butyl]-amide (compound 92)

### Step I

Boc-L-Ornithinol(Z) (1.13 g, 352.43 g/mol, 3.19 mmol, 1.0 eq.) was alkylated with 7-hydroxyisoquinoline (601 mg, 145.16 g/mol, 4.14 mmol) according to the procedure described in Example 40, step II. After silica gel chromatography purification 722 mg (47% yield) of (S)-5-(isoquinolin-6-yloxy)-4-N-Boc-1-N′-Z-pentane-1,4-diamine were obtained.

### Step II

The Boc protection was removed by treating (S)-5-(isoquinolin-6-yloxy)-4-N-Boc-1-N′-Z-pentane-1,4-diamine (722 mg, 479.58 g/mol, 1.5 mmol) with TFA according to the procedure described in Example 3, step V. After silica gel chromatographic purification 440 mg (77% yield) of (S)-5-(isoquinolin-6-yloxy)-1-N-Z-pentane-1,4-diamine were obtained.

### Step II

(S)-5-(isoquinolin-6-yloxy)-1-N-Z-pentane-1,4-diamine (285 mg, 379.46 g/mol, 0.75 mmol, 1.0 eq) was sulfonylated with 4-bromo-1-naphthalenesulfonyl chloride (345 mg, 305.58 g/mol, 1.13 mmol, 1.5 eq) according to the procedure described in Example 3, step IV. After silica gel chromatography purification 81 mg (17%) yield of (S)-4-bromo-naphthalene-1-sulfonic acid [4-(N-Z-amino)-1-(isoquinolin-6-yloxymethyl)-butyl]-amide was obtained.

### Step III

The Z-protection was removed by dissolving (S)-4-bromo-naphthalene-1-sulfonic acid [4-(N-Z-amino)-1-(isoquinolin-6-yloxymethyl)-butyl]-amide (250 mg, 648.58 g/mol, 0.53 mmol, 1.0 eq) in MeCN (1.0 ml), followed by the addition of iodotrimethylsilane (152 μl, 200.09 g/mol, 1.4 g/cm³, 1.1 mmol, 2.1 eq). After a reaction time of 1.5 hours the reaction mixture was evaporated to dryness. The residue was dissolved in DCM and washed with 10% Na₂S₂O₃. The organic phase was then dried over Na₂SO₄ and evaporated. The reaction product was purified by automated RP-LC chromatography. In this manner 6 mg (2% yield) of the title compound were obtained.

MS-ESI⁺ (m/z): 516

### Example 60

### Synthesis of (S)-4-methylnaphthalene-1-sulfonic acid [1-(benzylaminomethyl)-2-(1H-imidazol-4-yl)ethyl]amide (compound 93)

### Step I

Boc-L-His(DNP)—OH.IPA (0.963 g, 481.46 g/mol, 2.0 mmol, 1 eq, IRIS Biotech), DCC (0.495 g, 2.4 mmol, 1.2 eq) and HOBt (0.270 g, 2.0 mmol, 2 eq) were dissolved in 10 ml of DCM. Benzylamine (262 μl, 107.16 g/mol, 0.981 g/cm³, 2.4 mmol, 1.2 eq) was added, and the reaction mixture was stirred at room temperature overnight. The solvent was evaporated, and the residue was purified by chromatography to give crude (S)—N-benzyl-2-Boc-amino-3-(1-DNP-1H-imidazol-4-yl)propionamide.

### Step II

The Boc protection of (S)—N-benzyl-2-Boc-amino-3-(1-DNP-1H-imidazol-4-yl)propionamide (2.0 mmol) was removed as described in Example 6, step III. After evaporation of the solvent, the obtained crude (S)—N-benzyl-2-amino-3-(1-DNP-1H-imidazol-4-yl)propionamide trifluoroacetic acid salt was used without further purification in the next reaction step.

### Step III

The (S)—N-benzyl-2-amino-3-(1-DNP-1H-imidazol-4-yl)propionamide trifluoroacetic acid salt was sulfonylated according to the procedure described in Example I, step IV. After chromatographic purification, 540 mg (44% yield) of (S)—N-benzyl-2-(4-methylnaphthalene-1-sulfonylamino)-3-(1-DNP-1H-imidazol-4-yl)propionamide was obtained.

### Step IV

The DNP protection of (S)—N-benzyl-2-(4-methylnaphthalene-1-sulfonylamino)-3-(1-DNP-1H-imidazol-4-yl)propionamide (280 mg, 448.545 g/mol, 0.455 mmol) was removed by treating the compound with 20 vol-% piperidine in DMF at room temperature for 2 h. Solvents were evaporated and the residue was purified by chromatography. Thus, S)—N-benzyl-2-(4-methylnaphthalene-1-sulfonylamino)-3-(1H-imidazol-4-yl)propionamide was obtained with 78% yield in pure form.

### Step V

The S)—N-benzyl-2-(4-methylnaphthalene-1-sulfonylamino)-3-(1H-imidazol-4-yl)propionamide (50 mg, 434.562 g/mol, 0.113 mmol, 1 eq) was treated overnight with BTHF (1.0 M in THF, 4.52 ml, 4.52 mmol, 40 eq) in THF at room temperature. The reaction was quenched by adding methanol, the solvents were evaporated and the residue taken up in water/acetic acid (1:1, 4 ml). The reaction mixture was refluxed for 5 h and then stirred at room temperature overnight. The solvents were evaporated and the product was purified by chromatography. Thus, 25 mg (50% yield) of the title compound containing 0.5 equivalent of acetic acid was obtained.

MS-ESI⁺ (m/z): 435

¹H NMR (500 MHz, CD₃OD; δ, ppm): 8.61 (m, 1H), 8.14 (m, 1H), 8.11 (d, 1H), 7.64 (m, 2H), 7.42 (m, 1H), 7.29 (m, 3H), 7.17 (m, 3H), 6.36 (s, 1H), 3.68 (m, 2H), 3.62 (m, 1H), 2.76 (s, 3H), 2.74-2.64 (m, 2H), 2.55-2.44 (m, 2H), 1.94 (s, CH₃COOH).

### Example 61

### Synthesis of (S)-4-methylnaphthalene-1-sulfonic acid [2-(1H-imidazol-4-yl)-1-phenylaminomethylethyl]amide (compound 94)

The compound was synthesized according to the procedure described in Example 60, except that aniline instead of benzylamine was used. Thus, the title compound containing 0.2 equivalent acetic acid was obtained with 27% overall yield.

MS-ESI⁺ (m/z): 421

¹H NMR (500 MHz, CD₃OD; δ, ppm): 8.62 (m, 1H), 8.14 (m, 1H), 8.04 (d, 1H), 7.64 (m, 2H), 7.32 (m, 1H), 7.27 (s, 1H), 6.91 (m, 2H), 6.54 (s, 1H), 6.50 (m, 1H), 6.11 (d, 2H), 3.52 (m, 1H), 3.06-2.96 (m, 2H), 2.76 (m, 1H), 2.73 (s, 3H), 2.64 (m, 1H), 1.95 (s, CH₃COOH).

### Example 62

Additional compounds (including but not restricted to those described below) were prepared according to methods described in examples 1-33 but using the corresponding starting materials.

| | MS-ESI⁺ | |
|---|---|---|
| Name | (m/z) | Example |
| 4-methylnaphthalene-1-sulfonic acid (2-benzylamino-1- | 438 | 2 |
| piperidin-4-ylethyl)amide (compound 34) | | |
| (S)-4-methylnaphthalene-1-sulfonic acid [4-amino-1- | 412 | 2 |
| (benzylaminomethyl)butyl]amide (compound 35) | | |
| (R)-4-methylnaphthalene-1-sulfonic acid (1-carbamoyl- | 397 | 16 |
| methyl-3-phenylpropyl)amide (compound 36) | | |
| (R)-4-methylnaphthalene-1-sulfonic acid [1-carbamoyl- | 433 | 16 |
| methyl-2-(naphthalen-1-yl)ethyl]amide (compound 37) | | |
| (S)-4-methylnaphthalene-1-sulfonic acid [1-carbamoyl- | 433 | 16 |
| methyl-2-(naphthalen-1-yl)ethyl]amide (compound 38) | | |
| (S)-4-methylnaphthalene-1-sulfonic acid (2-amino-1- | 355 | 16 |
| benzylethyl)amide (compound 39) | | |
| (S)-4-methylnaphthalene-1-sulfonic acid [1-(2-amino- | 383 | 16 |
| ethyl)-3-phenylpropyl]amide (compound 40) | | |
| (S)-4-methylnaphthalene-1-sulfonic acid {[2-amino-1- | 419 | 16 |
| (naphthalen-1-yl)methyl]ethyl}amide (compound 41) | | |
| (R)-4-methylnaphthalene-1-sulfonic acid {[2-amino-1- | 419 | 16 |
| (naphthalen-1-yl)methyl]ethyl}amide (compound 42) | | |
| (S)-4-methylnaphthalene-1-sulfonic acid (1-aminomethyl- | 385 | 3 |
| 2-benzyloxyethyl)amide (compound 43) | | |
| (S)-4-methylnaphthalene-1-sulfonic acid [1-benzyloxy- | 482 | 18 |
| methyl-2-(4-methylpiperazin-1-yl)-2-oxoethyl]amide | | |
| (compound 44) | | |
| (S)-4-methylnaphthalene-1-sulfonic acid [1-(2-amino- | 442 | 24 |
| ethylcarbamoyl)-2-benzyloxyethyl]amide (compound 45) | | |
| (R)-4-methylnaphthalene-1-sulfonic acid {1-[(2-aminoethyl- | 428 | 24, 19 |
| amino)methyl]-2-benzyloxyethyl}amide (compound 46) | | |
| (R)-4-methylnaphthalene-1-sulfonic acid [1-benzyloxy- | 468 | 18, 19 |
| methyl-2-(4-methylpiperazin-1-yl)ethyl]amide (compound 47) | | |
| (S)-4-bromonaphthalene-1-sulfonic acid (4-amino-1- | 479 | 3 |
| benzyloxymethylbutyl)amide (compound 48) | | |
| 4-methylnaphthalene-1-sulfonic acid [3-benzyloxy-1-(1- | 495 | 3, 4 |
| guanidinylpiperidin-4-yl)propyl]amide (compound 49) | | |
| 4-methylnaphthalene-1-sulfonic acid [2-benzyloxy-1-(1- | 481 | 3, 4 |
| guanidinylpiperidin-4-yl)ethyl]amide (compound 50) | | |
| (R)-4-methylnaphthalene-1-sulfonic acid [1-benzylsulfa- | 498 | 18 |
| nylmethyl-2-(4-methylpiperazin-1-yl)-2-oxoethyl]amide | | |
| (compound 51) | | |
| (R)-4-methylnaphthalene-1-sulfonic acid [1-(4-methyl- | 512 | 18 |
| benzylsulfanylmethyl)-2-(4-methylpiperazin-1-yl)-2- | | |
| oxoethyl]amide (compound 52) | | |
| (R)-4-methylnaphthalene-1-sulfonic acid [1-(2-dimethyl- | 500 | 18 |
| aminoethylcarbamoyl)-2-(4-methylbenzyloxy)ethyl]amide | | |
| (compound 53) | | |
| (R)-4-methylnaphthalene-1-sulfonic acid [1-(4-methoxy- | 528 | 18 |
| benzylsulfanylmethyl)-2-(4-methylpiperazin-1-yl)-2-oxo- | | |
| ethyl]amide (compound 54) | | |
| (R)-4-methylnaphthalene-1-sulfonic acid [1-(2-dimethyl- | 516 | 18 |
| aminoethylcarbamoyl)-2-(4-methoxylbenzyloxy)ethyl]- | | |
| amide (compound 55) | | |
| (R)-4-methylnaphthalene-1-sulfonic acid [2-(4-methyl- | 498 | 18, 19 |
| benzylsulfanyl)-1-(4-methylpiperazin-1-ylmethyl)ethyl]- | | |
| amide (compound 56) | | |
| (R)-4-methylnaphthalene-1-sulfonic acid [2-(4-methoxy- | 514 | 18, 19 |
| benzylsulfanyl)-1-(4-methylpiperazin-1-ylmethyl)ethyl]- | | |
| amide (compound 57) | | |
| (R)-4-methylnaphthalene-1-sulfonic acid [1-[(2-dimethyl- | 502 | 18, 19 |
| aminoethylamino)methyl]-2-(4-methoxybenzyl- | | |
| sulfanyl)ethyl]amide (compound 58) | | |
| (R)-4-methylnaphthalene-1-sulfonic acid [2-(4-methoxy- | 556 | 18 |
| benzylsulfanyl)-2-methyl-1-(4-methylpiperazine-1- | | |
| carbonyl)propyl]amide (compound 59) | | |
| (R)-4-methylnaphthalene-1-sulfonic acid [1-(2-dimethyl- | 544 | 18 |
| aminoethylcarbamoyl)-2-(4-methoxybenzylsulfanyl)-2- | | |
| methylpropyl]amide (compound 60) | | |
| (S)-4-methylnaphthalene-1-sulfonic acid (1-benzylsulfanyl- | 471 | 1, 33 |
| methyl-4-isopropylaminobutyl)amide (compound 61) | | |
| (S)-4-methylnaphthalene-1-sulfonic acid (1-benzyloxy- | 455 | 12, 33 |
| methyl-4-isopropylaminobutyl)amide (compound 62) | | |
| (R)-4-methylnaphthalene-1-sulfonic acid (1-benzyloxy- | 413 | 16, steps I and II, |
| methyl-2-carbamoylethyl)amide (compound 63) | | 1, step III and IV |
| (R)-4-methylnaphthalene-1-sulfonic acid (3-amino-1- | 399 | 3 |
| benzyloxymethylpropyl)amide (compound 64) | | |
| (S)-benzo[b]thiophene-3-sulfonic acid [2-benzyloxy-1-(2- | 462 | 18 |
| dimethylaminoethylcarbamoyl)ethyl]amide (compound 66) | | |
| (S)-4-methylnaphthalene-1-sulfonic acid (4-amino-1- | 443 | 1 |
| phenethylsulfanylmethylbutyl)amide (compound 68) | | |
| (S)-Benzo[b]thiophene-3-sulfonic acid (4-amino-1- | 405 | 15 |
| benzyloxy-methylbutyl)amide (compound 95) | | |
| (S)-4-Methyl-naphthalene-1-sulfonic acid [4-amino-1- | 453 | 13 |
| (5,6,7,8-tetrahydronaphthalen-1-yloxymethyl)butyl]amide | | |
| (compound 96) | | |
| (S)-4-Bromo-naphthalene-1-sulfonic acid [4-isopropyl- | 557 | 13 and 10 |
| amino-1-(isoquinolin-6-yloxymethyl)butyl]amide | | |
| (compound 97) | | |
| 4-Methylnaphthalene-1-sulfonic acid [4-(benzylamino- | 424 | 2, I-III, V and |
| methyl)-piperidin-4-yl]amide (compound 98) | | IV |
| (S)-phenylmethanesulfonic acid (4-amino-1-benzyloxy- | 363 | 15 |
| methylbutyl)amide (compound 99) | | |
| (S)-4-Methylnaphthalene-1-sulfonic acid (3-amino-1- | 385 | 3, step I, 13 |
| phenoxymethylpropyl)amide (compound 100) | | |
| (S)-4-methylnaphthalene-1-sulfonic acid [4-amino-1-(4- | 431 | 15 |
| fluorobenzyloxymethyl)butyl]amide (compound 101) | | |
| (S)-4-methylnaphthalene-1-sulfonic acid (4-amino-1-penta- | 503 | 15 |
| fluorophenylmethoxymethylbutyl)amide (compound 102) | | |
| 4-Methylnaphthalene-1-sulfonic acid [1-benzyloxy- | 481 | 3, 4 |
| methyl-2-(1-guanidinylpyrrolidin-2-yl)ethyl]amide | | |
| (compound 103) | | |
| (R)-4-methylnaphthalene-1-sulfonic acid [1-(aminomethyl- | 424 | 38, 57 |
| carbamoyl)-4-phenylbut-3-enyl]amide (compound 104) | | |
| (S)-4-methylnaphthalene-1-sulfonic acid [4-isopropylamino- | 492 | 35, 33 |
| 1-(isoquinolin-6-yloxymethyl)butyl]amide (compound 105) | | |

### Example 63

### Binding Affinity at the Human Somatostatin Receptor Subtypes

The affinity of the compounds of the invention for the five human somatostatin receptor subtypes (sst₁, sst₂, sst₃, sst₄, and sst₅) was determined in competition binding assays with (¹²⁵I-Tyr)-[Leu⁸, DTrp²²]-Somatostatin-28 (¹²⁵I-LTT-SRIF-28). The biological material for these experiments consisted of membranes from Chinese hamster ovary (CHO) cells stably transfected with one of the five human somatostatin receptor subtypes. Membranes (3-20 μg of total protein per sample) and trace amount of ¹²⁵I-LTT-SRIF-28 were incubated in 10 mM Hepes, 1 mM EDTA, 5 mM MgCl₂, 5 mg/ml of BSA and 30 μg/ml bacitracin, pH 7.6, with six concentrations of the compounds. Each concentration was run in duplicate. Nonspecific binding was defined by 1 μM somatostatin-14 (SRIF-14) and corresponded to 5-25% of total binding. After 60 min at room temperature, incubations were terminated by rapid vacuum filtration through GF/B glass fiber filter mats (presoaked at 4° C. in 200 ml of 10 mM Hepes, 1 mM EDTA, 5 mM MgCl₂, pH 7.6) and three 5 ml washes with ice-cold wash buffer (20 mM TRIS, 1 mM EDTA, 5 mM MgCl₂, pH 7.4). The filters were then dried, impregnated with scintillate and their radioactivity was measured by scintillation counting. The analysis of the experiments was carried out by nonlinear least square curve fitting. Affinity constants (Kᵢ) were calculated from the IC₅₀ values according to the Cheng-Prusoff's equation (Cheng and Prusoff, 1973). Experiments were repeated a minimum of three times.

Using the aforementioned protocol, the following test results were obtained.

| | sst₁ | sst₂ | sst₃ | sst₄ | sst₅ |
|---|---|---|---|---|---|
| Compound | Kᵢ (nM) | Kᵢ (nM) | Kᵢ (nM) | Kᵢ (nM) | Kᵢ (nM) |
| compound 33 | 9.6 ± 0.5 | >10 000 | >3 000 | 110 ± 10 | >3 000 |
| compound 2 | 200 ± 60 | >10 000 | >3 000 | 5.6 ± 3.2 | >3 000 |

Besides these, a set of compounds of the invention had Kᵢ values of less than 300 nM for the sst₁. Among this set were for example:

compound 4

compound 13

compound 14

compound 15

compound 17

compound 27

compound 32

compound 33

compound 61

compound 62

compound 64

compound 69

compound 71

compound 79

compound 105.

Furthermore, another set of the compounds of the invention had Kᵢ values of less than 300 nM for the sst₄. Among this set were for example:

compound 1

compound 2

compound 3

compound 4

compound 12

compound 13

compound 15

compound 24

compound 27

compound 31

compound 32

compound 33

compound 34

compound 35

compound 62

compound 64

compound 67

compound 69

compound 70

compound 76

compound 77

compound 78

compound 79

compound 80

compound 83

compound 84

compound 85

compound 86

compound 87

compound 88

compound 93

compound 94

compound 95

compound 100

compound 101

compound 102.

### REFERENCES

Aavik et al. (2002), Elimination of vascular fibrointimal hyperplasia by somatostatin receptor 1,4-selective agonist FASEB J
van den Anker-Lugtenburg et al. (1996), Somatostatin receptor scintigraphy in the initial stageing of Hodgkin's disease. Br J Haematol 93:96-103
Bito et al. (1994), Functional coupling of SSTR4, a major hippocampal somatostatin receptor, to adenylate cyclase inhibition, arachidonate release and activation of the mitogen-activated protein kinase cascade. J Biol Chem 269:12722-12730
Cheng and Prusoff (1973), Relationship between the inhibition constant (KI) and the concentration of inhibitor which causes 50 percent inhibition (150) of an enzymatic reaction, Biochem. Pharmacol. 22:3099-3108
Curtis et al. (2000), Somatostatin receptor subtype expression and function in human vascular tissue. Am J Physiol Heart Circ Physiol 278:H1815-1822
Eriksen et al. (1995), Randomized double-blind Scandinavian trial of angiopeptin versus placebo for the prevention of clinical events and restenosis after coronary balloon angioplasty. Am Heart J 130:1-8
Fehlmann et al. (2000), Distribution and characterisation of somatostatin receptor mRNA and binding sites in the brain and periphery. J Physiol Paris 94:265-281
Haldemann et al. (1995), Somatostatin receptor scintigraphy in central nervous system tumors: role of blood-brain barrier permeability. J Nucl Med 36:403-410
Hoyer et al. (1995), Classification and nomenclature of somatostatin receptors. TIPS 16:86-88
Janson et al. (1994) [111In]-DPTA-D-Phe1]octreotide scintigraphy in patients with carcinoid tumours: the predictive value for somatostatin analogue treatment. Eur J Endocrinol 131:577-581
Mori et al. (1997), Differential expression of somatostatin receptors in the rat eye: SSTR4 is intensely expressed in the iris/ciliary body. Neurosci Left 223:185-188
Patel (1999), Somatostatin and its receptor family. Front Neuroendocrinol 20:157-198
Reisine and Bell (1995), Molecular biology of somatostatin receptors. Endocrinological Reviews 16:427-442
Reubi et al (1997), A selective analog for the somatostatin sst1-receptor subtype expressed by human tumors. Eur J Pharmacol 345:103-110
Reubi et al. (2001), Somatostatin receptor sst1-sst5 expression in normal and neoplastic human tissues using receptor autoradiography with subtype-selective ligands. Eur J Nucl Med 28:836-846
Reubi J C (2002), Peptide receptors as molecular targets for cancer diagnosis and therapy. Endocr Rev 24:389-427
Rivier et al. (2001), Potent somatostatin undecapeptide agonists selective for somatostatin receptor 1 (sst1). J Med Chem 44:2238-2246
Rohrer et al. (1998), Rapid identification of subtype-selective agonists of the somatostatin receptor through combinatorial chemistry. Science 282:737-740
Sinisi et al. (1997), Different expression patterns of somatostatin receptor subtypes in cultured epithelial cells from human normal prostate and prostate cancer. J Clin Endocrinol Metab 82:2566-2569
van Essen et al. (1997), Effects of octreotide treatment on restenosis after coronary angioplasty: results of the VERAS study. Circulation 96:1482-1487

1. A method of treating a disease or condition in mammals where an agonist or antagonist of somatostatin receptor subtypes 1 and/or 4 is indicated to be useful, comprising administering to a mammal that has said disease or condition a treatment effective amount of a compound of formula I 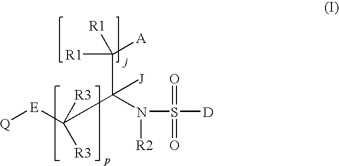
or pharmaceutically acceptable salt or ester thereof, wherein
A is NR6R6 or NR6-(C₁-C₃)alkyl-NR6R6 and the (C₁-C₃)alkyl may be unsubstituted or substituted with one to four groups selected from R^{a}; or
A is a 5- to 6-membered saturated or unsaturated ring containing 0 to 2 nitrogens, said ring being unsubstituted or substituted with 1 to 3 groups independently selected from R6, except R6 being H, and (CH₂)ₛ—NR6R6; or
A and J together with the carbon atom to which they are attached form a 5- to 6-membered ring containing 1 to 2 nitrogens, said ring being unsubstituted or substituted with 1 to 3 groups independently selected from R6 or —(CH₂)ₛ—NR6R6; or
A and J together with the carbon atom to which they are attached form a 5- to 6-membered ring containing 0 nitrogens, said ring being substituted by a group —(CH₂)ₛ—NR6R6 and 0 to 2 groups independently selected from R6; or
A and R2 together with the atoms to which they are attached form a saturated 5- or 6-membered ring, said ring being substituted by a group —(CH₂)ₛ—NR6R6 and 0 to 3 groups independently selected from (C₁-C₆)alkyl;
D is aryl, heteroaryl or aryl-(C₁-C₂)-alkyl and may be unsubstituted or substituted with one to seven groups selected from R^{a};
E is O, S, NR^{b}, or CR^{b}R^{b};
J is H or methyl; or J is part of a spiro ring system together with A;
Q is
1. aryl,
2. heteroaryl or
3. a group of formula R4R5CH—;
wherein aryl or heteroaryl is unsubstituted or substituted with 1 to 4 substituents selected from R^{a};
R1 is independently
a group selected from R^{a}; or
R1 and R1 together form ═O,
R2 is
1. H,
2. (C₁-C₆)alkyl,
3. (C₂-C₆)alkenyl,
4. (C₃-C₇)cycloalkyl, or
5. benzyl
or R2 is part of a ring system together with A;
R3 is independently
1. H,
2. (C₁-C₆)alkyl, or
when E is NR^{b} or CR^{b}R^{b}, R3 and R^{b} may form a double bond between the atoms to which they are attached;
R4 is
1. H,
2. (C₁-C₆)alkyl,
3. (C₂-C₆)alkenyl,
4. (C₂-C₆)alkynyl,
5. Cy,
6. Cy-(C₁-C₆)alkyl or
7. Cy-(C₂-C₆)alkenyl,
wherein alkyl, alkenyl, alkynyl and Cy are each optionally substituted with one to two substituents selected from R^{a};
R5 is
1. H,
2. (C₁-C₆)alkyl,
3. (C₂-C₆)alkenyl,
4. (C₂-C₆)alkynyl
5. aryl,
6. aryl-(C₁-C₆)alkyl,
7. heteroaryl,
8. heteroaryl-(C₁-C₆)alkyl,
9. —OR^{b};
10. —(CH₂)ₖ—OR^{b} or
11. —(CH₂)ₖC(O)NHR^{b},
wherein aryl and heteroaryl are each optionally substituted with one to two substituents selected from R^{a}; or
R4 and R5 together with the atom to which they are attached form a 3- to 7-membered ring containing 0 to 2 heteroatoms selected from N, O and S, wherein said ring may be substituted with one to three substituents selected from R^{a}; or said ring may be fused to aryl or heteroaryl which may be substituted with one to three substituents selected from R^{a};
R6 is independently
1. H,
2. (C₁-C₆)alkyl,
3. (C₃-C₇)cycloalkyl,
4. (C₃-C₇)cycloalkyl(C₁-C₆)alkyl or
5. —C(═NR^{b})NR^{b}R^{b},
wherein symbols R^{b} together may form a 5- to 6-membered unsaturated or saturated ring; or
R6 and R6 together with the atoms to which they are attached form a 5- to 7-membered ring containing 1 to 3 heteroatoms selected from N, O and S, said ring being unsubstituted or substituted with 1 to 4 groups independently selected from (C₁-C₆)alkyl or halogen;
R^{a} is independently
1. H,
2. halogen,
3. —OR^{b},
4. —(C₁-C₆)alkyl-OR^{b},
5. (C₁-C₆)alkyl,
6. —CF₃,
7. —NO₂,
8. —SR^{b},
9. —NR^{b}R^{b},
10. —CN,
11. —C(O)R^{b},
12. (C₂-C₆)alkenyl,
13. (C₃-C₇)cycloalkyl
14. —NR^{b}C(O)R^{b} or
15. —C(O)NHR^{b}.
R^{b} is independently
1. hydrogen,
2. (C₁-C₆)alkyl,
3. Cy or
4. Cy-(C₁-C₄)alkyl;
p is an integer 0 to 3;
j is an integer 0 to 4;
k is an integer 0 to 2,
s is an integer 0 to 2; and
Cy is cycloalkyl, heterocyclyl, aryl or heteroaryl;
with the proviso that when
a) E is CR^{b}R^{b} or NR^{b}, then R1 and R1 cannot together form ═O,
b) A is pyrrole or pyrazole, one of the 1 to 3 substituents on said ring must be selected from —C(═NR^{b})NR^{b}R^{b}, —(CH₂)ₛ—NR6-C(═NR^{b})NR^{b}R^{b} or —(CH₂)ₛ—NR6R6,
c) A is a 6-membered unsaturated ring not containing a nitrogen atom, said ring must be substituted with 1 to 3 substituents selected from —C(═NR^{b})NR^{b}R^{b}, —(CH₂)ₛ—NR6-C(═NR^{b})NR^{b}R^{b} or —(CH₂)ₛ—NR6R6,
d) A is a saturated ring not containing a nitrogen atom, at least one of the 1 to 3 substituents on ring A must be selected from —C(═NR^{b})NR^{b}R^{b}, —(CH₂)ₛ—NR6-C(═NR^{b})NR^{b}R^{b} or —(CH₂)ₛ—NR6R6.

2. The method according to claim 1, where the compound is an agonist.

3. The method according to claim 1, where the compound is an antagonist.

4. The method according to claim 1, where the compound is sst₁ selective.

5. The method according to claim 1, where the compound is sst₄ selective.

6. The method according to claim 1, wherein the compound of Formula I is a compound of Formula II 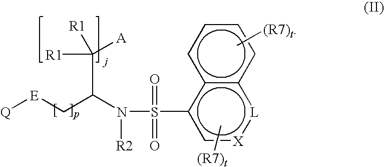
or a pharmaceutically acceptable salt or ester thereof,
R2 is H or CH₃;
R7 is independently
1) H,
2) halogen,
3) —NO₂,
4) —NR^{b}R^{b},
5) —CN,
6) —OR^{b},
7) —SR^{b},
8) —C(O)R^{b},
9) (C₁-C₆)alkyl,
10) (C₂-C₆)alkenyl,
11) (C₃-C₇)cycloalkyl or
12) —CF₃;
L is C(R7), S or N;
X is a bond or C(R7); and
t is an integer from 0 to 7.

7. The method according to claim 6, wherein
L is CR7;
X is CH;
R7 is selected from H, (C₁-C₄)alkyl or halogen and t=1.

8. The method according to claim 1, wherein
E is O or NR^{b};
R3 is H and
p is 1 or 2.

9. The method according to claim 1, wherein
R1 is H;
p is 1 or 2;
j is 2 or 3; and
A is NR6R6, with R6 chosen from H, (C₁-C₃)alkyl or —C(═NH)NH₂.

10. The method according to claim 6, wherein
R1 is H;
p is 1 or 2;
j is 2 or 3;
A is NR6R6, with R6 chosen from H, (C₁-C₃)alkyl or —C(═NH)NH₂;
R7 is H, halogen or —(C₁-C₃)alkyl; and
t is 0 or 1.

11. The method according to claim 1, wherein
R1 is H;
j is 0 or 1;
A is 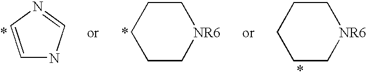
with the star denoting the point of attachment and
R6 is H, (C₁-C₃)alkyl or —C(═NH)NH₂.

12. The method according to claim 1, wherein
J is 1;
A is NR6-(C₁-C₃)alkyl-NR6R6 and R6 is selected independently from H, (C₁-C₄)alkyl, cyclopropyl or —C(═NH)NH₂

13. The method according to claim 1, wherein R3 is H and p is 1.

14. The method according to claim 1, wherein the compound is (S)-4-methylnaphthalene-1-sulfonic acid (1-benzyloxymethyl-3-guanidinylpropyl)amide, (S)-4-methylnaphthalene-1-sulfonic acid (4-amino-1-phenoxymethylbutyl)amide, (S)-4-methylnaphthalene-1-sulfonic acid (4-amino-1-benzyloxymethylbutyl)amide, 4-methylnaphthalene-1-sulfonic acid (2-benzylamino-1-piperidin-4-ylethyl)amide, (S)-4-methylnaphthalene-1-sulfonic acid (4-isopropylamino-1-phenoxymethylbutyl)amide, (S)-4-methylnaphthalene-1-sulfonic acid (4-amino-1-benzylsulfanylmethylbutyl)amide, (R)-4-methylnaphthalene-1-sulfonic acid [1-(2-aminoethylcarbamoyl)-2-benzyloxyethyl]amide, (S)-4-methylnaphthalene-1-sulfonic acid [1-(2-aminoethylcarbamoyl)-2-benzylsulfanylethyl]amide, (S)-4-methylnaphthalene-1-sulfonic acid {2-benzyloxy-1-[(2-dimethylaminoethylamino)methyl]ethyl}amide, (S)—N-(1-benzyloxymethyl-3-guanidinylpropyl)-2,3,4,5,6-pentamethylbenzenesulfonamide, (S)-4-methylnaphthalene-1-sulfonic acid {4-isopropylamino-1-[(1,2,3,4-tetrahydronaphthalen-1-ylamino)methyl]butyl}amide, (R)-[4-methylnaphthalene-1-sulfonic acid [1-(2-aminoethyl)-3-phenylpropyl]amide, (S)-4-methyl-naphthalene-1-sulfonic acid [1-(benzylamino-methyl)-2-(1H-imidazol-4-yl)-ethyl]amide, (S)-4-methylnaphthalene-1-sulfonic acid [2-(1H-imidazol-4-yl)-1-phenoxymethylethyl]amide, (S)-4-methylnaphthalene-1-sulfonic acid [1-benzyloxymethyl-3-(4,5-dihydro-1H-imidazol-2-ylamino)propyl]amide or (S)-4-methylnaphthalene-1-sulfonic acid [2-(1H-imidazol-4-yl)-1-phenylaminomethylethyl]-amide.

15. The method according to claim 1, where the disease or condition is at least one member selected from the group consisting of depression, anxiety, bipolar disorders, ADHD, angiogenesis, restenosis, new blood vessel sprouting, arteriosclerosis, diabetic angiopathy, diabetic retinopathy, cancerous tumors and tumor metastasis, high intraocular pressure, age-related macular degeneration, wound healing, acute forms of neurogenic and non-neurogenic inflammation and pain, chronic inflammation and neuropathic pain.

16. A method for imaging of healthy or diseased tissues and/or organs possessing sst₁ and/or sst₄ receptors, comprising administering to said tissues and/or organs and a compound of Formula I or pharmaceutically acceptable salt or ester thereof, and imaging said tissues and/or organs; 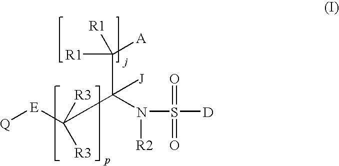
wherein
A is NR6R6 or NR6-(C₁-C₃)alkyl-NR6R6 and the (C₁-C₃)alkyl may be unsubstituted or substituted with one to four groups selected from R^{a}; or
A is a 5- to 6-membered saturated or unsaturated ring containing 0 to 2 nitrogens, said ring being unsubstituted or substituted with 1 to 3 groups independently selected from R6, except R6 being H, and —(CH₂)ₛ—NR6R6; or
A and J together with the carbon atom to which they are attached form a 5- to 6-membered ring containing 1 to 2 nitrogens, said ring being unsubstituted or substituted with 1 to 3 groups independently selected from R6 or —(CH₂)ₛ—NR6R6; or
A and J together with the carbon atom to which they are attached form a 5- to 6-membered ring containing 0 nitrogens, said ring being substituted by a group —(CH₂)ₛ—NR6R6 and 0 to 2 groups independently selected from R6; or
A and R2 together with the atoms to which they are attached form a saturated 5- or 6-membered ring, said ring being substituted by a group —(CH₂)ₛ—NR6R6 and 0 to 3 groups independently selected from (C₁-C₆)alkyl;
D is aryl, heteroaryl or aryl-(C₁-C₂)-alkyl and may be unsubstituted or substituted with one to seven groups selected from R^{a};
E is O, S, NR^{b}, or CR^{b}R^{b};
J is H or methyl; or J is part of a spiro ring system together with A;
Q is
1. aryl,
2. heteroaryl or
3. a group of formula R4R5CH—;
wherein aryl or heteroaryl is unsubstituted or substituted with 1 to 4 substituents selected from R^{a};
R1 is independently
a group selected from R^{a}; or
R1 and R1 together form ═O,
R2 is
1. H,
2. (C₁-C₆)alkyl,
3. (C₂-C₆)alkenyl,
4. (C₃-C₇)cycloalkyl, or
5. benzyl
or R2 is part of a ring system together with A;
R3 is independently
1. H,
2. (C₁-C₆)alkyl, or
when E is NR^{b} or CR^{b}R^{b}, R3 and R^{b} may form a double bond between the atoms to which they are attached;
R4 is
1. H,
2. (C₂-C₆)alkyl,
3. (C₂-C₆)alkenyl,
4. (C₂-C₆)alkynyl,
5. Cy,
6. Cy-(C₁-C₆)alkyl or
7. Cy-(C₂-C₆)alkenyl,
wherein alkyl, alkenyl, alkynyl and Cy are each optionally substituted with one to two substituents selected from R^{a};
R5 is
1. H,
2. (C₁-C₆)alkyl,
3. (C₂-C₆)alkenyl,
4. (C₂-C₆)alkynyl
5. aryl,
6. aryl-(C₁-C₆)alkyl,
7. heteroaryl,
8. heteroaryl-(C₁-C₆)alkyl,
9. —OR^{b};
10. —(CH₂)ₖ—OR^{b} or
11. —(CH₂)ₖC(O)NHR^{b},
wherein aryl and heteroaryl are each optionally substituted with one to two substituents selected from R^{a}; or
R4 and R5 together with the atom to which they are attached form a 3- to 7-membered ring containing 0 to 2 heteroatoms selected from N, O and S, wherein said ring may be substituted with one to three substituents selected from R^{a}; or said ring may be fused to aryl or heteroaryl which may be substituted with one to three substituents selected from R^{a};
R6 is independently
1. H,
2. (C₁-C₆)alkyl,
3. (C₃-C₇)cycloalkyl,
4. (C₃-C₇)cycloalkyl(C₁-C₆)alkyl or
5. —C(═NR^{b})NR^{b}R^{b},
wherein symbols R^{b} together may form a 5- to 6-membered unsaturated or saturated ring; or
R6 and R6 together with the atoms to which they are attached form a 5- to 7-membered ring containing 1 to 3 heteroatoms selected from N, O and S, said ring being unsubstituted or substituted with 1 to 4 groups independently selected from (C₁-C₆)alkyl or halogen;
R^{a} is independently
1. H,
2. halogen,
3. —OR^{b},
4. —(C₁₋₆)alkyl-OR^{b},
5. (C₁-C₆)alkyl,
6. —CF₃,
7. —NO₂,
8. —SR^{b},
9. —NR^{b}R^{b},
10. —CN,
11. —C(O)R^{b},
12. (C₂-C₆)alkenyl,
13. (C₃-C₇)cycloalkyl
14. —NR^{b}C(O)R^{b} or
15. —C(O)NHR^{b}.
R^{b} is independently
1. hydrogen,
2. (C₁-C₆)alkyl,
3. Cy or
4. Cy-(C₁-C₄)alkyl;
p is an integer 0 to 3;
j is an integer 0 to 4;
k is an integer 0 to 2,
s is an integer 0 to 2; and
Cy is cycloalkyl, heterocyclyl, aryl or heteroaryl;
with the proviso that when
a) E is CR^{b}R^{b} or NR^{b}, then R1 and R1 cannot together form ═O,
b) A is pyrrole or pyrazole, one of the 1 to 3 substituents on said ring must be selected from —C(═NR^{b})NR^{b}R^{b}, —(CH₂)ₛ—NR6-C(═NR^{b})NR^{b}R^{b} or —(CH₂)ₛ—NR6R6,
c) A is a 6-membered unsaturated ring not containing a nitrogen atom, said ring must be substituted with 1 to 3 substituents selected from —C(═NR^{b})NR^{b}R^{b}, —(CH₂)ₛ—NR6-C(═NR^{b})NR^{b}R^{b} or —(CH₂)ₛ—NR6R6,
d) A is a saturated ring not containing a nitrogen atom, at least one of the 1 to 3 substituents on ring A must be selected from —C(═NR^{b})NR^{b}R^{b}, —(CH₂)ₛ—NR6-C(═NR^{b})NR^{b}R^{b} or —(CH₂)ₛ—NR6R6.

17. A compound of Formula I′, 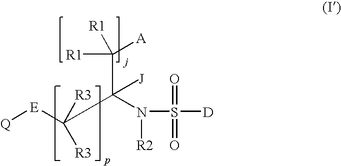
or a pharmaceutically acceptable salt or ester thereof, wherein
A is NR6R6 or NR6-(C₁-C₃)alkyl-NR6R6 and the (C₁-C₃)alkyl may be unsubstituted or substituted with one to four groups selected from R^{a}; or
A is a 5- to 6-membered saturated or unsaturated ring containing 0 to 2 nitrogens, said ring being unsubstituted or substituted with 1 to 3 groups independently selected from R6, except R6 being H, and —(CH₂)ₛ—NR6R6; or
A and J together with the carbon atom to which they are attached form a 5- to 6-membered ring containing 1 to 2 nitrogens, said ring being unsubstituted or substituted with 1 to 3 groups independently selected from R6 or —(CH₂)s-NR6R6; or
A and J together with the carbon atom to which they are attached form a 5- to 6-membered ring containing 0 nitrogens, said ring being substituted by a group —(CH₂)ₛ—NR6R6 and 0 to 2 groups independently selected from R6; or
A and R2 together with the atoms to which they are attached form a saturated 5- or 6-membered ring, said ring being substituted by a group —(CH₂)ₛNR6R6 and 0 to 3 groups independently selected from (C₁-C₆)alkyl;
D is aryl, heteroaryl or aryl-(C₁-C₂)-alkyl and may be unsubstituted or substituted with one to seven groups selected from R^{a};
E is O, S or NR^{b};
J is H or methyl; or J is part of a spiro ring system together with A;
Q is
1. phenyl
2. benzyl or
4. a group of formula R4R5CH—;
wherein the phenyl or benzyl is unsubstituted or substituted with 1 to 4 substituents selected from R^{a};
R1 is independently
a group selected from R^{a};
R2 is
1. H,
2. (C₁-C₆) alkyl,
3. (C₂-C₆)alkenyl,
4. (C₃-C₇)cycloalkyl, or
5. benzyl
or R2 is part of a ring system together with A;
R3 is independently
1) H,
2) (C₁-C₆) alkyl, or
when E is NR^{b}, R3 and R^{b} may form a double bond between the atoms to which they are attached;
R4 is
1. H,
2. (C₁-C₆)alkyl,
3. (C₂-C₆)alkenyl,
4. (C₂-C₆)alkynyl,
5. Cy,
6. Cy-(C₁-C₆)alkyl or
7. Cy-(C₂-C₆)alkenyl,
wherein alkyl, alkenyl, alkynyl and Cy are each optionally substituted with one to two substituents selected from R^{a};
R5 is
1. H,
2. (C₁-C₆)alkyl,
3. (C₂-C₆)alkenyl,
4. (C₂-C₆)alkynyl
5. aryl,
6. aryl-(C₁-C₆)alkyl,
7. heteroaryl,
8. heteroaryl-(C₁-C₆)alkyl,
9. —OR^{b},
10. —(CH₂)ₖ—OR^{b} or
11. —(CH₂)ₖC(O)NHR^{b},
wherein aryl and heteroaryl are each optionally substituted with one to two substituents selected from R^{a}; or
R4 and R5 together with the atom to which they are attached form a 3- to 7-membered ring containing 0 to 2 heteroatoms selected from N, O and S, wherein said ring may be substituted with one to three substituents selected from R^{a}; or said ring may be fused to aryl or heteroaryl which may be substituted with one to three substituents selected from R^{a};
R6 is independently
1. H,
2. (C₁-C₆)alkyl,
3. (C₃-C₇)cycloalkyl,
4. (C₃-C₇)cycloalkyl(C₁-C₆)alkyl or
5. —C(═NR^{b})NR^{b}R^{b},
wherein symbols R^{b} together may form a 5- to 6-membered unsaturated or saturated ring; or
R6 and R6 together with the atoms to which they are attached form a 5- to 7-membered ring containing 1 to 3 heteroatoms selected from N, O and S, said ring being unsubstituted or substituted with 1 to 4 groups independently selected from (C₁-C₆)alkyl or halogen;
R^{a} is independently
1. H,
2. halogen,
3. —OR^{b},
4. —(C₁-C₆)alkyl-OR^{b},
5. (C₁-C₆)alkyl,
6. —CF₃,
7. —NO₂,
8. —SR^{b},
9. —NR^{b}R^{b},
10. —CN,
11. —C(O)R^{b},
12. (C₂-C₆)alkenyl,
13. (C₃-C₇)cycloalkyl
14. —NR^{b}C(O)R^{b} or
15. —C(O)NHR^{b};
R^{b} is independently
1. hydrogen,
2. (C₁-C₆) alkyl,
3. Cy or
4. Cy-(C₁-C₄)alkyl;
p is an integer 0 to 3;
j is an integer 0 to 4;
k is an integer 0 to 2;
s is an integer 0 to 2; and
Cy is cycloalkyl, heterocyclyl, aryl or heteroaryl;
with the proviso that when
a) A contains an aromatic system, then E cannot be O;
b) E is NR^{b} and A is NR6R6 then p and j cannot simultaneously be 1,
c) A is pyrrole or pyrazole, one of the 1 to 3 substituents on said ring must be selected from —C(═NR^{b})NR^{b}R^{b}, —(CH₂)₅—NR6-C(═NR^{b})NR^{b}R^{b} or —(CH₂)ₛ—NR6R6,
d) A is a 6-membered unsaturated ring not containing a nitrogen atom, said ring must be substituted with 1 to 3 substituents selected from —C(═NR^{b})NR^{b}R^{b}, —(CH₂)ₛ—NR6-C(═NR^{b})NR^{b}R^{b} or —(CH₂)ₛ—NR6R6,
e) A is a saturated ring not containing a nitrogen atom, at least one of the 1 to 3 substituents on ring A must be selected from —C(═NR^{b})NR^{b}R^{b}, —(CH₂)ₛ—NR6-C(═NR^{b})NR^{b}R^{b} or —(CH₂)ₛ—NR6R6.

18. A compound according to claim 17, which is a compound of formula II′. 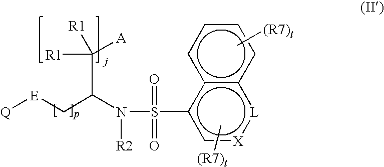
wherein
R2 is H or CH₃;
R7 is independently
1) H,
2) halogen,
3) —NO₂,
4) —NR^{b}R^{b},
5) —CN,
6) —OR^{b},
7) —SR^{b},
8) C(O)R^{b},
9) (C₁-C₆)alkyl,
10) (C₂-C₆)alkenyl,
11) (C₃-C₇)cycloalkyl or
12) CF₃;
L is C(R7), S or N;
X is a bond or C(R7);
p is 1 or 2 and
t is an integer from 0 to 7.

19. A compound according to claim 17, wherein E is O or NR^{b}.

20. A compound according to claim 17, wherein
A is NR6R6 with R6 is selected independently from H, (C₁-C₄)alkyl, cyclopropyl or —C(═NH)NH₂; and
j is 2 or 3.

21. A compound according to claim 17, wherein
R1 is H;
j is 0 or 1; and
A is 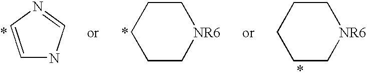
with the star denoting the point of attachment and
R6 is H, (C₁-C₃)alkyl or —C(═NH)NH₂.

22. A compound according to claim 17, wherein Q is phenyl or benzyl unsubstituted or substituted with 1 to 4 substituents selected from R^{a}.

23. A compound according to claim 18, wherein L is C(R7), t is 0 or 1, X is CH and R7 is selected from H, (C₁-C₃)alkyl or halogen.

24. A compound according to claim 18, wherein E is O or NR^{b}.

25. A compound according to claim 18, wherein
A is NR6R6 and R6 is selected independently from H, (C₁-C₄)alkyl, cyclopropyl or —C(═NH)NH₂; and
j is 2 or 3.

26. A compound according to claim 18, wherein
A is NR6-(C₁-C₃)alkyl-NR6R6 and R6 is selected independently from H, (C₁-C₄)alkyl, cyclopropyl or —C(═NH)NH₂; and
j is 1.

27. A compound according to claim 18, wherein
R1 is H;
j is 0 or 1 and
A is 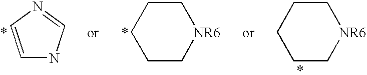
with the star denoting the point of attachment and
R6 is H, (C₁-C₃)alkyl or —C(═NH)NH₂.

28. A compound according to claim 18, wherein
Q is phenyl or benzyl unsubstituted or substituted with 1 to 4 substituents selected from R^{a}.

29. A compound according to claim 25, wherein
L is C(R7), t is 0 or 1, X is CH and R7 is selected from H, (C₁-C₃)alkyl or halogen.

30. A compound according to claim 25, wherein E is O or NR^{b}.

31. A compound according to claim 25, wherein Q is phenyl or benzyl unsubstituted or substituted with 1 to 4 substituents selected from R^{a}.

32. A compound of Formula I′ according to claim 17, wherein the compound is (S)-4-methylnaphthalene-1-sulfonic acid (1-benzyloxymethyl-3-guanidinylpropyl)amide, (S)-4-methylnaphthalene-1-sulfonic acid (4-amino-1-phenoxymethylbutyl)amide, (S)-4-methyl-naphthalene-1-sulfonic acid (4-amino-1-benzyloxymethylbutyl)amide[J=2607], 4-methyl-naphthalene-1-sulfonic acid (2-benzylamino-1-piperidin-4-ylethyl)amide, (S)-4-methylnaphthalene-1-sulfonic acid(4-isopropylamino-1-phenoxymethyl-butyl)amide, (S)-4-methylnaphthalene-1-sulfonic acid (4-amino-1-benzyl-sulfanylmethylbutyl)amide, (S)-4-methylnaphthalene-1-sulfonic acid {2-benzyl-oxy-1-[(2-dimethylaminoethylamino)methyl]ethyl}amide, (S)—N-(1-benzyloxy-methyl-3-guanidinylpropyl)-2,3,4,5,6-pentamethylbenzenesulfonamide, (S)-4-methylnaphthalene-1-sulfonic acid {4-isopropylamino-1-[(1,2,3,4-tetrahydro-naphthalen-1-ylamino)methyl]butyl}amide, (R)-[4-methylnaphthalene-1-sulfonic acid [1-(2-aminoethyl)-3-phenylpropyl]amide, (S)-4-methylnaphthalene-1-sulfonic acid [1-(benzylamino-methyl)-2-(1H-imidazol-4-yl)ethyl]amide, (S)-4-methylnaphthalene-1-sulfonic acid [1-benzyloxymethyl-3-(4,5-dihydro-1H-imidazol-2-ylamino)propyl]amide or (S)-4-methylnaphthalene-1-sulfonic acid [2-(1H-imidazol-4-yl)-1-phenylaminomethylethyl]amide.

33. A process for preparing a compound as claimed in claim 18, comprising reacting an amidated amino acid of Formula III, 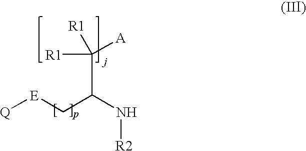
wherein R2 is H, alkyl, cycloalkyl or a protecting group, with a sulfonyl acid derivative of Formula IV, 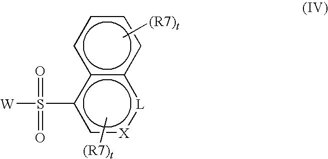
wherein W is OH or a halogen, and where the compounds of Formula III and IV are optionally protected.

34. A pharmaceutical composition comprising a compound of Formula I′ according to claim 17 as an active ingredient together with a pharmaceutically acceptable diluent, carrier and/or excipient.
